(19) United States
(12) Patent Application Publication
(10) Pub. No.: US 2007/0105826 A1
(43) Pub. Date: May 10, 2007

(54) Novel fusidic acid derivatives

(75) Inventors: Tore Duvold, Thorvaldsensvej (DK); Claus Aage Svensgaard Bretting, Smallegade (DK); Poul Rodbroe Rasmussen, Harevej (DK); Laetitia Maud Elysa Bouerat Duvold, Thorvaldsensvej (DK); Jacob Thorhauge, Drosselvaenget (DK)

Correspondence Address: BIRCH STEWART KOLASCH & BIRCH, PO BOX 747, FALLS CHURCH, VA, 22040-0747, US

(73) Assignee: LEO PHARMA A/S, Ballerup (DK)

(21) Appl. No.: 10/563,103

(22) PCT Filed: Jul. 9, 2004
(86) PCT No.: PCT/DK04/00491

§ 371 Date: Dec. 30, 2005

Related U.S. Application Data
(60) Provisional application No. 60/487,609, filed on Jul. 17, 2003.

(30) Foreign Application Priority Data
Jul. 16, 2003 (DK) PA 2003 01080
Oct. 9, 2003 (DK) PA 2003 01486

(51) Int. Cl. A61K 31/655; C07J 9/00; A61K 31/56

(52) U.S. Cl. 514/169; 552/540; 514/151; 552/1

(57) ABSTRACT

Fusidic acid derivatives substituted at C-24 may be used in therapy for the treatment of infections.

### FIELD OF THE INVENTION

The present invention relates to novel fusidic acid derivatives, to pharmaceutical compositions comprising said derivatives, as well as to their use in therapy.

### BACKGROUND OF THE INVENTION

Fusidic acid belongs to the fusidanes which is a small family of naturally occurring antibiotics.

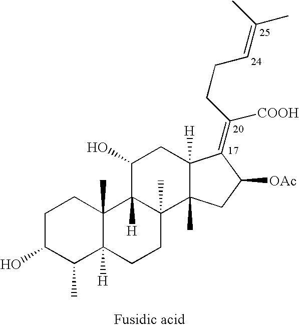

The fusidanes have in common a tetracyclic ring system with a unique chair-boat-chair conformation, which distinguishes them from steroids. Therefore, in spite of some structural similarity with steroids, namely a tetracyclic system, the fusidanes do not exert any hormonal activity. The fusidanes also have in common a carboxylic acid bearing side chain linked to the ring system at C-17 via a double bond and an acetate group linked at C-16. Fusidic acid, a fermentation product of Fusidium coccineum, is the most antibiotically active compound of the fusidanes and is the only fusidane used clinically in treatment of infectious diseases. Fusidic acid (Fucidin®) is used clinically for the treatment of severe staphylococcal infections, particularly in bone and joint infections, in both the acute and the intractable form of the disease (The Use of Antibiotics, 5^{th} Ed., A. Kucers and N. McK. Bennett (Eds.), Butterworth 1997, pp. 580-587, and references cited therein). Although fusidic acid is most commonly used against staphylococci, It is also used against several other gram-positive species. The clinical value of fusidic acid Is also due to Its efficient distribution In various tissues, low degree of toxicity and allergic reactions and the absence cross-resistance with other clinically used antibiotics. Fusidic acid is widely used in local therapy for a number of skin and eye infections caused by staphylococci. It is generally given in combination with common antibiotics such as penicillins, erythromycins or clindamycin. It has also been used as an alternative to vancomycin for the control of Clostridium difficile. Compared to staphylococci, several other gram-positive cocci are often less susceptible to fusidic acid. As an example, streptococcal species are generally up to 100-fold less sensitive to fusidic acid than staphylococci [Kuchers et al; supra]. Other sensitive bacteria Include gram-positive anaerobic cocci, such as Peptococcus and Peptostreptococcus spp., aerobic or anaerobic gram-positive bacteria, such as Corynebacterium diphtheriae, Clostridium tetani, Clostridium difficile and Clostridium perfringens. Gram-negative bacteria are resistant except for Neisseria spp. and Legionella pneumophila. The drug is highly potent against both intracellular and extracellular M. leprae. The structure-activity relationship (SAR) of fusidic acid has been extensively studied and a large number of analogues have been prepared. However, only a few of these analogues have shown activities comparable with that of fusidic acid. In spite of the extensive SAR studies, the potential of side chain modifications has not extensively been explored.

Compared to other antibiotics, fusidic acid has so far not developed serious clinical problems with drug resistance [Turnidge, Int. J. Antimicro. Agents, 12, S35-S44, 1999]. However, as discussed above the substance in itself has a fairly limited antibiotic spectrum, and it might therefore be desirable to develop novel analogues based on fusidic acid with an antibiotic activity against a broader range of pathogenic microorganisms, and in particular streptococci.

Attempts to improve the therapeutic properties of fusidanes by manipulating the side chain have previously been made. Thus, WO 02/070537 discloses fusidic acid derivatives wherein the C17-C20 double bond has been converted to a cyclopropane moiety by introduction of a methylene group.

WO 01/29061 discloses fusidic acid derivatives wherein the C17-C20 double bond has been saturated.

### SUMMARY OF THE INVENTION

The present Inventors have surprisingly found that fusidic acid derivatives wherein C-24 is substituted retain the activity against staphylococci and significantly increase the activity against streptococci. Accordingly, the present invention relates to compounds of general formula I 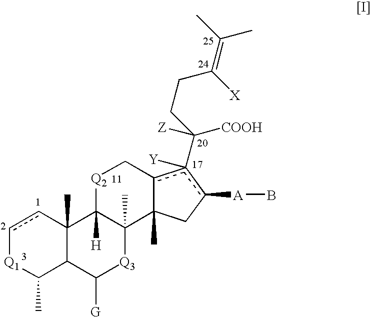
wherein X represents halogen, trifluoromethyl, cyano, azido, alkyl, alkenyl or aryl, wherein said alkyl, alkenyl or aryl are optionally substituted by one or more, same or different substituents selected from the group consisting of alkyl, alkenyl, aryl, alkoxy, nitro, alkylthio, halogen, azido, trifluoromethyl and cyano;

Y and Z both represent hydrogen, or together with the C-17/C-20 bond form a double bond between C-17 and C-20, or together are methylene and form a cyclopropane ring in combination with C-17 and C-20;

A represents a bond, O, S or S(O);

B represents C₁₋₆ alkyl, C₂₋₆ alkenyl, C₁₋₆ acyl, C₃₋₇ cycloalkylcarbonyl or benzoyl, all of which are optionally substituted with one or more substituents selected from the group consisting of halogen, hydroxy, alkoxy, aryl, heteroaryl and azido, or, if A represents a bond, B may also represent hydrogen;

Q₁ and Q₂ Independently represent —CH₂—, —C(O)—, —(CHOH)—, —(CHOR)—, —(CHSH)—, —(NH)—,

—(CHNH₂)— or —(CHW)—, wherein R represents C₁₋₆alkyl and W represents halogen, cyano, azido or trifluoromethyl;

Q₃ represents —CH₂—, —C(O)— or —CHOH—;

G represents hydrogen, OH or O—CO—CH₃;

two bonds in the pentacyclic ring being depicted with full and dotted lines to indicate that either of the two bonds may be a double bond, in which case Y is absent and Z represents hydrogen;

the bond between C-1 and C-2 being either a single or a double bond;

and pharmaceutically acceptable salts and easily hydrolysable esters thereof.

In another aspect, the invention relates to compounds of formula I for use in therapy, and in particular to pharmaceutical composition comprising a compound according to formula I together with a pharmaceutically acceptable excipient or vehicle.

In a further aspect, the invention relates to a method of treating, preventing or ameliorating infections, the method comprising administering an effective amount of a compound according to formula I to a patient in need thereof.

In a still further aspect, the invention relates to the use of compounds according to formula I for the manufacture of a medicament for the prevention, treatment, amelioration or prophylaxis of infections.

In a still further aspect, the invention relates to the use of compounds according to formula I for controlling microbial growth and for the prevention or prophylaxis of bacterial infections during animal breeding.

In a still further aspect, the invention relates to a method of preparing a compound of formula Ia 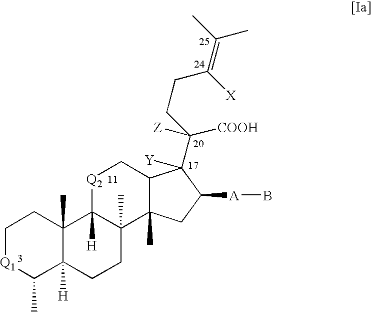
wherein X represents bromo, Y and Z both represent hydrogen, or together with the C-17/C-20 bond form a double bond between C-17 and C-20, or together are methylene and form a cyclopropane ring in combination with C-17 and C-20; A represents a bond, O, S or S(O); B represents C₁₋₆ alkyl, C₂₋₆ alkenyl, C₁₋₆ acyl, C₃₋₇ cycloalkylcarbonyl or benzoyl, all of which are optionally substituted with one or more substituents selected from the group consisting of halogen, hydroxy, C₁₋₆ alkoxy, aryl, heterocyclyl and azido, or, if A represents a bond, B may also represent hydrogen; Q₁ and Q₂ independently represent —C(O)—, —(CHOH)—, —(CHSH)—, or —(CHW)—, wherein W represents halogen, cyano, azido or trifluoromethyl, the method comprising

(a) dissolving fusidic acid or a suitable fusidic acid analogue In a suitable organic solvent followed by treatment with bromine to give a 24,25-dibromo intermediate of general structure Ib, 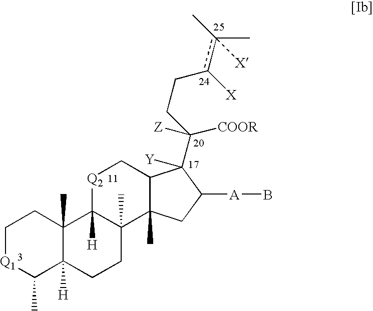
wherein X and X′ represent bromo, R is hydrogen, the bond between C-24 and C-25 is a single bond, and Y, Z, A, B, Q₁, and Q₂ are as defined above;

(b) treating a solution of the 24,25-dibromo intermediate in a suitable solvent in the presence of a suitable base to give the dehydrobrominated compound of formula Ia, in the form of a salt, and

(c) acidifying the salt generated in step (b) to obtain the compound of formula Ia in free acid form.

In a still further aspect, the invention relates to a compound of general structure Ib, 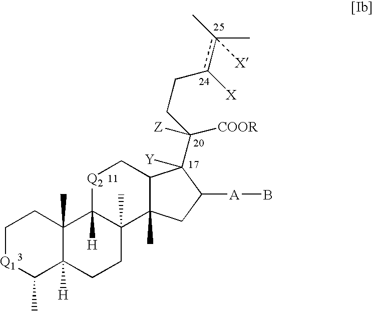
wherein X and X′ represent bromo, R is hydrogen, the bond between C-24 and C-25 is a single bond, Y and Z both represent hydrogen, or together with the C-17/C-20 bond form a double bond between C-17 and C-20, or together are methylene and form a cyclopropane ring in combination with C-17 and C-20, A represents a bond, O, S or S(O); B represents C₁₋₆ alkyl, C₂₋₆ alkenyl, C₁₋₆ acyl, C₃₋₇ cycloalkylcarbonyl or benzoyl, all of which are optionally substituted with one or more substituents selected from the group consisting of halogen, hydroxy, C₁₋₆ alkoxy, aryl, heterocyclyl and azido, or, if A represents a bond, B may also represent hydrogen; Q₁ and Q₂ independently represent —C(O)—, —(CHOH)—, —(CHSH)—, or —(CHW)—, wherein W represents halogen, cyano, azido or trifluoromethyl.

### DETAILED DESCRIPTION OF THE INVENTION

Definitions

In the present context, the term “alkyl” is intended to indicate a univalent radical derived from an alkane by removal of a hydrogen atom from any carbon atom, and includes the subclasses of primary, secondary and tertiary alkyl groups, including for example C₁-C₁₂ alkyl, such as C₁-C₈ alkyl, such as C₁-C₆ alkyl, such as C₁-C₄ alkyl, methyl, ethyl, n-propyl, isopropyl, n-butyl, isobutyl, sec-butyl, t-butyl, pentyl, hexyl, nonyl, dodecanyl, cyclopropyl, cyclopropylmethyl, cyclobutyl, cyclopentyl and cyclohexyl. Alkane refers to an acyclic or cyclic, branched or unbranched saturated hydrocarbon and therefore consisting entirely of hydrogen atoms and carbon atoms.

The term “alkenyl” is intended to indicate to a straight or branched acyclic hydrocarbon having one or more carbon-carbon double bonds of either E or Z stereochemistry where applicable. The term includes, for example, C₂-C₁₂ alkenyl, C₂-C₈ alkenyl, C₂-C₆ alkenyl, vinyl, allyl, 1-butenyl, 2-butenyl, and 2-methyl-2-propenyl.

The term “acyl” is intended to indicate a radical of the formula —CO—R, wherein R is alkyl as defined above, for example C₁-C₆ acyl, such as acyl, propionyl, butyryl, or pivaloyl.

The term “alkoxy” is intended to Indicate a radical of the formula —OR, wherein R is alkyl as defined above, for example C₁-C₅ alkoxy, C₁-C₃ alkoxy, methoxy, n-propoxy, t-butoxy, and the like.

The term “halogen” indicates a member of the seventh main group of the periodical system, i.e. fluoro, chloro, bromo, and iodo.

The term “cycloalkylcarbonyl” is intended to indicate a radical of the formula —C(O)—R′, wherein R′ represents a cyclic alkyl as indicated above.

The term “aryl” is Intended to indicate a cyclic, optionally a fused bicyclic, radical, wherein all ring atoms are carbon, and wherein the ring is aromatic, or in the case of a fused ring system, at least one ring is aromatic. Examples of aryl include phenyl, napthyl and tetralinyl.

The term “alkylthio” is intended to indicate a radical of the formula —S—R′, wherein R′ is alkyl as indicated above.

The term “heteroaryl” is intended to include radicals of heterocyclic aromatic rings comprising 1-6 heteroatoms (selected from O, S and N) and 1-20 carbon atoms, such as 1-5 heteroatoms and 1-10 carbon atoms, such as 1-5 heteroatoms and 1-6 carbon atoms, such as 1-5 heteroatoms and 1-3 carbon atoms, in particular 5- or 6-membered rings with 1-4 heteroatoms selected from O, S and N, or optionally fused bicyclic rings with 1-4 heteroatoms, and wherein at least one ring is aromatic, e.g. furanyl, pyridyl, quinolyl, isoquinolyl, indolyl, tetrazolyl, thiazolyl, imidazolyl, pyrazolyl, oxazolyl, Isoxazolyl, thienyl, pyrazinyl, isothiazolyl, benzimidazolyl and benzofuranyl.

The expression “easily hydrolysable esters” is used In this specification to denote alkanoyloxyalkyl, aralkanoyloxyalkyl, aroyloxyalkyl, for example acetoxymethyl, pivaloyloxymethyl, benzoyloxymethyl esters and the corresponding 1′-oxyethyl derivatives, or alkoxycarbonyloxyalkyl esters, for example methoxycarbonyloxymethyl esters and ethoxycarbonyloxymethyl esters, and the corresponding 1′-oxyethyl derivatives, or lactonyl esters, for example phthalidyl esters, or dialkylaminoalkyl esters, for example diethylaminoethyl esters. The expression “easily hydrolysable esters” includes in vivo hydrolysable esters of the compounds of the invention. Such esters may be prepared using methods known to a skilled person in the art, cf. GB patent No. 1 490 852 hereby incorporated by reference.

### Preferred Embodiments of Compounds of Formula I

In a preferred embodiment, the invention relates to compounds of general formula Ia 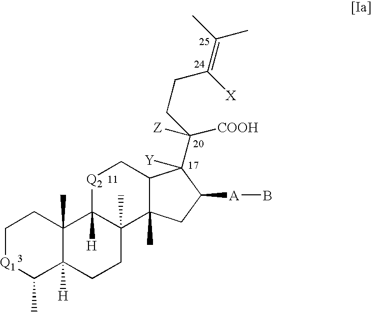
wherein X represents halogen, trifluoromethyl, cyano, azido, C₁₋₇ alkyl, C₂₋₉ alkenyl or aryl, wherein said C₁₋₆ alkyl, C₂₋₆ alkenyl or aryl are optionally substituted by one or more, same or different substituents selected from the group consisting of C₁₋₇ alkyl, C₂₋₉ alkenyl, aryl, C₁₋₆ alkoxy, nitro, alkylthio, halogen, azido, trifluoromethyl and cyano;

Y and Z both represent hydrogen, or together with the C-17/C-20 bond form a double bond between C-17 and C-20, or together are methylene and form a cyclopropane ring in combination with C-17 and C-20;

A represents a bond, O, S or S(O);

B represents C₁₋₆ alkyl, C₂₋₆ alkenyl, C₁₋₆ acyl, C₃₋₇ cycloalkylcarbonyl or benzoyl, all of which are optionally substituted with one or more substituents selected from the group consisting of halogen, hydroxy, C₁₋₆ alkoxy, aryl, heteroaryl and azido, or, if A represents a bond, B may also represent hydrogen;

Q₁ and Q₂ Independently represent —C(O)—, —(CHOH)—, —(CHSH)—, or —(CHW)—, wherein W represents halogen, cyano, azido or trifluoromethyl;

and pharmaceutically acceptable salts and easily hydrolysable esters thereof.

In a preferred embodiment of compounds of formula I, Ia or Ib, Y and Z are both hydrogen, and the stereochemical configuration is S at both C-17 and C-20.

In another preferred embodiment of compounds of formula I, Ia or Ib, Y and Z together are methylene and form a cyclopropane ring in combination with C-17 and C-20, and the stereochemical configuration is S at both C-17 and C-20.

In yet another preferred embodiment of compounds of formula I, Ia, or Ib, Y and Z together with the C-17/C-20 bond form a double bond between C-17 and C-20. Most preferably the configuration of the double bond between C-17 and C-20 is the same as in fusidic acid.

In yet another preferred embodiment X represents chloro, bromo, lodo, fluoro, methyl, ethyl, propyl, phenyl, vinyl, propenyl, butenyl, pentenyl, hexenyl, heptenyl, nonenyl, biphenyl or naphthyl, wherein said methyl, ethyl, propyl, phenyl, vinyl, propenyl, butenyl, pentenyl, hexenyl, heptenyl, nonenyl, biphenyl or naphthyl, are optionally substituted by one or more, same or different substituents selected from the group consisting of fluoro, chloro, bromo, phenyl, vinyl, cyano, methoxy, trifluoromethyl, nitro, methylthio, butyl, methyl, ethyl, propyl, butyl, pentyl, hexyl, and heptyl.

In a particular preferred embodiment X represents fluoro, chloro, bromo, iodo, trifluoromethyl, phenyl, 4-bromophenyl, 4-chlorophenyl, 3,5-difluorophenyl, trans-1-hexen-1-yl, trans-1-buten-3,3-dimethyl-1-yl, trans-1-nonen-1-yl, trans-5-chloro-1-penten-1-yl, trans-2-phenyl-1-vinyl, 2-phenyl-1-ethyl, 4-n-propylphenyl, 4-vinylphenyl, 4-tert-butylphenyl, 4-cyanophenyl, 3-biphenyl, 4-(trifluoromethyl)phenyl, 4-methoxyphenyl, 3-cyanophenyl, 2-methoxyphenyl, 3-nitrophenyl, 3-bromophenyl, 4-(methylthio)phenyl, 2-naphtyl, 3,5-bis-(trifluoromethyl)phenyl, 3,4-dimethoxyphenyl or 3,5-dibromophenyl.

Q₁ and Q₂ may advantageously be selected from the group consisting of —(CO)— and —(CHOH)—. Q1 may also advantageously represent CHF, CHCl, CHBr, CHI, CHN₃.

A still further embodiment of the invention provides compounds of formula Ia, wherein Q₁ and Q₂ both represent a

—(CHOH)— group, or one of Q₁ or Q₂ represents —(CO)—, or Q₁ represents CHF, CHCl, CHBr, CHI or CHN₃;

X represents chloro, bromo, iodo, trifluorometyl, azido or cyano;

Z and Y together with the C-17/C-20 bond form a double bond between C-17 and C-20;

A represents O, S or S(O);

B represents a C₁₋₄ alkyl group, optionally substituted with one or more substituents selected from the list consisting of azido, hydroxy, fluoro, chloro and bromo, or B represents a C₁₋₄ acyl group or a benzoyl group, both optionally substituted with one or more halogen atoms, such as e.g. fluoro and chloro.

In an particular preferred embodiment A represents O or S(O).

In yet another preferred embodiment B represents acyl, methyl, ethyl, propyl, butyl, pentyl, propenyl or cyclopentyl, all of which are optionally substituted with one or more substituents selected from the list consisting of methyl, ethyl, propyl, butyl, fluoro, vinyl, hydroxy, phenyl, furfuryl and methoxy.

In an particular preferred embodiment B is acetyl, isopropyl, ethyl, 2,2,2-trifluoroethyl, vinyl, 1-pentyl, 2-methyl-1-butyl, 3-methyl-1-butyl, cyclopentyl, 2-hydroxyethyl, benzyl, furfuryl, phenyl, 2-fluoroethyl, 2-methoxyethyl, 2,2,2-trichloroethyl, 2-azidoethyl, 2-hydroxyethyl, propyl, tert.-butyl, 1,3-difluoro-isopropyl, propionyl, chloroacetyl or trifluoroacetyl.

In an particular preferred embodiment Q₁ or Q₂ or both Q₁ and Q₂ represent —(COH)—.

When Q₁ and/or Q₂ In formulas I, Ia, or Ib represent —(COH)—, the stereochemical configuration is preferably 3α and 11α, respectively.

Specific examples of compounds of the invention are

24-trifluoromethyl fusidic acid sodium salt (Compound 101),

24-trifluoromethyl fusidic acid pivaloyloxymethyl ester (Compound 102),

24-chloro-fusidic acid (Compound 103),

24-chloro-fusidic acid pivaloyloxymethyl ester (Compound 104),

24-chloro-fusidic acid sodium salt (Compound 105),

24-trifluoromethyl fusidic acid (Compound 106),

24-bromo-fusidic acid acetoxymethyl ester (Compound 107),

24-bromo-fusidic acid (Compound 108),

24-bromo-fusidic acid sodium salt (Compound 109),

24-bromo-fusidic acid pivaloyloxymethyl ester (Compound 110),

24-bromo-16-deacetoxy-16β-thioacetyl-fusidic acid acetoxymethylester (Compound 111),

24-bromo-16-deacetoxy-16β-isopropylthio-fusidic acid (Compound 112),

24-bromo-16-deacetoxy-16β-isopropylsulfinyl-fusidic acid (Compound 113),

24-bromo-16-deacetoxy-16β-thioacetyl-fusidic acid (Compound 114),

24-bromo-17S,20S-dihydrofusidic acid (Compound 115),

24-bromo-16-deacetoxy-16β-ethoxy-fusidic acid (Compound 116),

24-bromo-16-deacetoxy-16β-ethoxy-fusidic acid acetoxymethyl ester (Compound 117),

24-bromo-16-deacetoxy -16β-(2′,2′,2′-trifluoroethoxy)-fusidic acid acetoxymethyl ester (Compound 118),

24-bromo-16-deacetoxy -16β-(2′,2′,2′-trifluoroethoxy)-fusidic acid (Compound 119),

24-bromo-17S,20S-fusidic acid acetoxymethyl ester (Compound 120),

24-bromo-17S,20S-methylene-fusidic acid acetoxymethyl ester (Compound 121),

24-bromo-17S,20S-methylene-fusidic acid (Compound 122),

3-deoxy-3β,24-dibromo-fusidic acid (Compound 123),

3α-azido-24-bromo-3-deoxy-fusidic acid (Compound 124),

24-iodo-fusidic acid (Compound 125),

24-iodo-fusidic acid acetoxymethyl ester (Compound 126),

24-iodo-fusidic acid pivaloyloxymethyl ester (Compound 127),

24-phenyl-fusidic acid pivaloyloxymethylester (Compound 136),

24-phenyl-fusidic acid (Compound 137),

24-(4-bromophenyl)-fusidic acid pivaloyloxymethylester (Compound 138),

24-(4-bromophenyl)-fusidic acid (Compound 139),

24-(4-chlorophenyl)-fusidic acid pivaloyloxymethylester (Compound 140),

24-(4-chlorophenyl)-fusidic acid (Compound 141),

24-(3,5-difluorophenyl)-fusidic acid pivaloyloxymethylester (Compound 142),

24-(3,5-difluorophenyl)-fusidic acid (Compound 143),

3-deoxy-3β,24-dibromo-fusidic acid acetoxymethyl ester (Compound 144),

24-bromo-16-deacetoxy-16β-ethylthio-fusidic acid (Compound 146),

24-bromo-16-deacetoxy-16β-ethylsulfinyl-fusidic acid (Compound 147),

24-bromo-16-deacetoxy-16β-allylthio-fusidic acid (Compound 148),

24-bromo-16-deacetoxy-16β-(1-pentylthio)-fusidic acid (Compound 149),

24-bromo-16-deacetoxy-16β-(1-pentylsulfinyl)-fusidic acid (Compound 150),

24-bromo-16-deacetoxy-16β-(2-methyl-1-butylthio)-fusidic acid (Compound 151),

24-bromo-16-deacetoxy-16β-(2-methyl-1-butylsulfinyl)-fusidic acid (Compound 152),

24-bromo-16-deacetoxy-16β-(3-methyl-1-butylthio)-fusidic acid (Compound 153),

24-bromo-16-deacetoxy-16β-(3-methyl-1-butylsulfinyl)-fusidic acid (Compound 154),

24-bromo-16-deacetoxy-16β-cyclopentylthio-fusidic acid (Compound 155),

24-bromo-16-deacetoxy-16β-(2,2,2-trifluoroethylthio)-fusidic acid (Compound 156),

24-bromo-16-deacetoxy-16β-(2-hydroxyethylthio)-fusidic acid (Compound 157),

24-bromo-16-deacetoxy-16β-benzylthio-fusidic acid (Compound 158),

24-bromo-16-deacetoxy-16β-benzylsulfinyl-fusidic acid (Compound 159),

24-bromo-16-deacetoxy-16β-(2-furylmethylthio)-fusidic acid (Compound 160),

24-bromo-16-deacetoxy-16β-phenylthio-fusidic acid (Compound 161),

24-bromo-16-deacetoxy-16β-benzoylthio-fusidic acid (Compound 162),

24-bromo-16-deacetoxy-16β-isopropoxy-fusidic acid (Compound 163),

24-bromo-16-deacetoxy-16β-(2-fluoroethoxy)-fusidic acid (Compound 164),

24-bromo-16-deacetoxy-16β-(2-methoxyethoxy)-fusidic acid (Compound 165),

24-(trans-1-hexen-1-yl)-fusidic acid (Compound 166),

24-(trans-1-buten-3,3-dimethyl-1-yl)-fusidic acid (Compound 167),

24-(trans-1-nonen-1-yl)-fusidic acid (Compound 168),

24-(trans-5-chloro-1-penten-1-yl)-fusidic acid (Compound 169),

24-(trans-2-phenyl-1-vinyl)-fusidic acid (Compound 170),

24-(2-phenyl-1-ethyl)-fusidic acid (Compound 171),

24-(4-n-propylphenyl)-fusidic acid (Compound 172),

24-(4-vinylphenyl)-fusidic acid (Compound 173),

24-(4-tert-butylphenyl)-fusidic acid (Compound 174),

24-(4-cyanophenyl)-fusidic acid (Compound 175),

24-(3-biphenyl)-fusidic acid (Compound 176),

24-(4-(trifluoromethyl)phenyl)-fusidic acid (Compound 177),

24-(4-methoxyphenyl)-fusidic acid (Compound 178),

24-(3-cyanophenyl)-fusidic acid (Compound 179),

24-(2-methoxyphenyl)-fusidic acid (Compound 180),

24-(3-nitrophenyl)-fusidic acid (Compound 181),

24-(3-bromophenyl)-fusidic acid (Compound 182),

24-(4-(methylthio)phenyl)-fusidic acid (Compound 183),

24-(2-naphtyl)-fusidic acid (Compound 184),

24-(3,5-bis-(trifluoromethyl)phenyl)-fusidic acid (Compound 185),

24-(3,4-dimethoxyphenyl)-fusidic acid (Compound 186),

24-(3,5-dibromophenyl)-fusidic acid (Compound 187),

24-bromofusidic acid, cholin salt (Compound 188),

24-bromofusidic acid, L-arginine salt (Compound 189),

24-bromofusidic acid, 2-(dimethylamino)-ethanol salt (Compound 190),

24-bromofusidic acid, 4-(2-hydroxyethyl)-morpholin salt (Compound 191),

24-bromofusidic acid, L-lysine salt (Compound 192),

24-bromofusidic acid, N-(2-hydroxyethyl)-pyrrolidine salt (Compound 193),

24-bromofusidic acid, ethanolamine salt ( Compound 194),

24-bromofusidic acid, potassium salt (Compound 195),

24-bromofusidic acid, tetrabutylammonium salt (Compound 196),

24-bromofusidic acid, benzyltrimethylammonium salt (Compound 197),

24-bromofusidic acid, cetyltrimethylammonium salt (Compound 198),

24-bromofusidic acid, tetramethylammonium salt (Compound 199),

24-bromofusidic acid, tetrapropylammonium salt (Compound 300),

24-bromofusidic acid, tris(hydroxymethyl)aminomethane salt (Compound 301),

24-bromofusidic acid, N-methyl-D-glucamine salt (Compound 302),

24-bromofusidic acid, silver salt (Compound 303),

24-bromofusidic acid, benzethonium salt (Compound 304),

24-bromofusidic acid, triethanolamine salt (Compound 305),

24-(trans-1-hexen-1-yl)-fusidic acid pivaloyloxymethylester (Compound 306), with 24-(trans-1-buten-3,3-dimethyl-1-yl)-fusidic acid pivaloyloxymethyl ester (Compound 307),

24-(trans-1-nonen-1-yl)-fusidic acid pivaloyloxymethyl ester (Compound 308),

24-(trans-5-chloro-1-penten-1-yl) -fusidic acid pivaloyloxymethyl ester (Compound 309),

24-(trans-2-phenyl-1-vinyl)-fusidic acid pivaloyloxymethyl ester (Compound 310),

24-(2-phenyl-1-ethyl)-fusidic acid pivaloyloxymethyl ester (Compound 311),

24-(4-n-propylphenyl)-fusidic acid pivaloyloxymethyl ester (Compound 312),

24-(4-vinylphenyl)-fusidic acid pivaloyloxymethyl ester (Compound 313),

24-(4-tert-butylphenyl)-fusidic acid pivaloyloxymethyl ester (Compound 314),

24-(4-cyanophenyl)-fusidic acid pivaloyloxymethyl ester (Compound 315),

24-(3-biphenyl)-fusidic acid pivaloyloxymethyl ester (Compound 316),

24-(4-(trifluoromethyl)phenyl)-fusidic acid pivaloyloxymethyl ester (Compound 317),

24-(4-methoxyphenyl)-fusidic acid pivaloyloxymethyl ester (Compound 318),

24-(3-cyanophenyl)-fusidic acid pivaloyloxymethyl ester (Compound 319),

24-(2-methoxyphenyl)-fusidic acid pivaloyloxymethyl ester (Compound 320),

24-(3-nitrophenyl)-fusidic acid pivaloyloxymethyl ester (Compound 321),

24-(3-bromophenyl)-fusidic acid pivaloyloxymethyl ester (Compound 322),

24-(4-(methylthio)phenyl)-fusidic acid pivaloyloxymethyl ester (Compound 323),

24-(2-naphtyl)-fusidic acid pivaloyloxymethyl ester (Compound 324),

24-(3,5-bis-(trifluoromethyl)phenyl)-fusidic acid pivaloyloxymethyl ester (Compound 325),

24-(3,4-dimethoxyphenyl)-fusidic acid pivaloyloxymethyl ester (Compound 326), and

24-(3,5-dibromophenyl)-fusidic acid pivaloyloxymethyl ester (Compound 327).

The compounds of the invention can be used as such or In the form of salts or easily hydrolysable esters (as defined above). In particular, salts of the present compounds are pharmaceutically acceptable salts, such as alkali metal salts and alkaline earth metal salts, for example sodium, potassium, magnesium or calcium salts, as well as silver salts and salts with bases, such as ammonia or suitable non-toxic amines, such as lower alkylamines, for example triethylamine, hydroxy-lower alkylamines, for example 2-hydroxyethylamine, bis-(2-hydroxyethyl)-amine, cycloalkylamines, for example dicyclohexylamine, or benzylamines, for example N,N′-dibenzylethylene-diamine, and dibenzylamine. The silver salts of the compounds may be especially useful for local treatment.

In a presently preferred embodiment pharmaceutically acceptable salts of a compound according to general formula I or Ia, are selected from the group consisting of sodium salts, choline salts, L-arginine salts, 2-(dimethylamino)-ethanol salts, 4-(2-hydroxyethyl)-morpholin salts, L-lysine salts, N-(2-hydroxyethyl)-pyrrolidine salts, ethanolamine salts, potassium salts, tetrabutylammonium salts, benzyltrimethylammonium salts, cetyltrimethylammonium salts, tetramethylammonium salts, tetrapropylammonium salts, tris(hydroxymethyl)aminomethane salts, N-methyl-D-glucamine salts, silver salts, benzethonium salts, and triethanolamine salts.

In a preferred embodiment of preparing a compound of formula Ia the solvent used in step (a) to dissolve the fusidic acid is acetic acid or a C₁₋₃ alkyl ester of a C₁₋₄ carboxylic acid, and in particular ethyl acetate.

In a preferred embodiment of preparing a compound of formula Ia the solvent used in step (b) to dissolve the 24,25-dibromo intermediate is a C₁₋₆ alcohol, such as methanol, ethanol, n-propanol, isopropanol or butanol, or water, or mixtures of said solvents.

In a preferred embodiment of preparing a compound of formula Ia the base used in step (b) to dehydrobrominate the 24,25-dibromo intermediate is an alkali metal or alkaline earth metal salt of a weak acid, such as carbonic, phosphoric or boric acid, e.g. potassium or sodium carbonate, or a base such as ammonia or C₁₋₈ substituted ammonia, e.g. ethylamine, diethylamine, triethylamine or piperidine, or an alkali or alkaline earth metal hydroxide such as dilute sodium hydroxide, calcium hydroxide or dilute potassium hydroxide.

The compounds of the present invention may comprise chiral carbon atom(s) and carbon-carbon double bond(s) which give rise to stereoisomeric forms. The present invention relates to all such isomers, either in pure form or as mixtures thereof. Pure stereoisomeric forms of the compounds of the invention may be obtained by the application of procedures known in the art. Diastereomers may be separated by physical separation methods such as selective crystallization and chromatographic techniques, e.g. liquid chromatography using chiral stationary phases. Said pure stereoisomeric forms may also be derived from the corresponding pure stereolsomeric forms of the appropriate starting materials, provided that the reaction occurs stereoselectively or stereospecifically. Preferably if a specific stereoisomer is desired, said compound will be synthesized by stereoselective or stereospecific methods of preparation.

Compounds of the present invention are useful for treating, preventing or ameliorating infections in a patient, including a mammalian, and in particular, a human patient. Animals that may be treated with a compound of the invention include, more specifically, domestic animals such as horses, cows, pigs, sheep, poultry, fish, cats, dogs and zoo animals. Compounds of the present invention may be particularly useful in the treatment of bacterial infections, such as skin infections or secondary skin infections, or eye infections. Compounds of the present invention may be furthermore useful in the treatment of simple abscesses, impetiginous lesions, furuncles, or cellulites. Compounds of the present invention may be particularly useful for the treatment, e.g. the topical treatment, of contagious superficial infections of the skin, such as non-bullous impetigo (or impetigo contagiosa) or bullous impetigo. Consequently, the present invention provides a method of treating, preventing or ameliorating bacterial infections, the method comprising administering to a patient an effective amount of a compound of formula I, optionally together with another therapeutically active compound. Examples of said other therapeutically active compounds include antibiotics, such as β-lactams, such as penicillins (phenoxymethyl penicillin, benzyl penicillin, dicloxacillin, ampicillin, amoxicillin, pivampicillin, flucloxacillin, piperacillin and mecellinam), cefalosporins (cefalexin, cefalotin, cefepim, cefotaxim, ceftazidim, ceftriazon and cefuroxim), monobactams (aztreonam) and carbapenems (meropenem); macrolides (azithromycin, clarithromycin, erythromycin and roxithromycin); polymyxins (colistin); tetracyclins (tetracycline, doxycyclin, oxytetracyclin and lymecyclin); aminoglycosides (streptomycin, gentamicin, tobramycin and netilmicin); fluoroquinolones (norfloxacin, ofloxacin, ciprofloxacin and moxifloxacin); clindamycin, lincomycin, teicoplanin, vancomycin, oxazolidones (linezolid), rifamycin, metronidazol and fusidic acid. Other compounds which may advantageously be combined with a compound of the Invention, especially for topical treatment, include for instance corticosteroids, such as hydrocortisone, betamethasone-17-valerate and triamcinolone acetonid. The compounds may either be administered concomitantly or sequentially.

Compounds of the present invention are further useful for the prevention or prophylaxis of bacterial infections in animals and are therefore useful during the breeding of domestic animals, such as mammals, such as horses, cows, pigs, sheep, poultry, fish, cats, dogs and zoo animals.

For use in therapy, compounds of the present invention are typically in the form of a pharmaceutical composition. The invention therefore relates to a pharmaceutical composition comprising a compound of formula I or Ia, optionally together with other therapeutically active compounds, together with a pharmaceutically acceptable excipient or vehicle. The excipient must be “acceptable” in the sense of being compatible with the other ingredients of the composition and not deleterious to the recipient thereof.

Conveniently, the active ingredient comprises from 0.05-99.9% by weight of the formulation.

In the form of a dosage unit, the compound may be administered one or more times a day at appropriate intervals, always depending, however, on the condition of the patient, and in accordance with the prescription made by the medical practitioner. Conveniently, a dosage unit of a formulation contain between 50 mg and 5000 mg, preferably between 200 mg and 1000 mg of a compound of formula I or Ia.

In the context of topical treatment it may be more appropriate to refer to “usage unit”, which denotes a single dose which is capable of being administered to a patient, and which may be readily handled and packed, remaining as a physically and chemically stable unit dose comprising either the active material as such or a mixture of it with solid or liquid pharmaceutical diluents or carriers.

The term “usage unit” in connection with topical use means a unitary, i.e. a single dose capable of being administered topically to a patient in an application per square centimetre of the infected area of from 0.1 mg to 10 mg and preferably from 0.2 mg to 1 mg of the active ingredient in question.

It is also envisaged that In certain treatment regimes, administration with longer intervals, e.g. every other day, every week, or even with longer intervals may be beneficial.

If the treatment involves administration of another therapeutically active compound it is recommended to consult Goodman & Gilman's The Pharmacological Basis of Therapeutics, 9^{th} Ed., J. G. Hardman and L. E. Limbird (Eds.), McGraw-Hill 1995, for useful dosages of said compounds.

The formulations include e.g. those in a form suitable for oral (including sustained or timed release), rectal, parenteral (including subcutaneous, intraperitoneal, Intramuscular, intraarticular and intravenous), transdermal, ophthalmic, topical, nasal or buccal administration.

The formulations may conveniently be presented in dosage unit form and may be prepared by any of the methods well known in the art of pharmacy, e.g. as disclosed in Remington, The Science and Practice of Pharmacy, 20^{th} ed., 2000. All methods include the step of bringing the active ingredient into association with the carrier, which constitutes one or more accessory ingredients. In general, the formulations are prepared by uniformly and Intimately bringing the active ingredient into association with a liquid carrier or a finely divided solid carrier or both, and then, if necessary, shaping the product into the desired formulation.

Formulations of the present invention suitable for oral administration may be in the form of discrete units as capsules, sachets, tablets or lozenges, each containing a predetermined amount of the active ingredient; in the form of a powder or granules; in the form of a solution or a suspension In an aqueous liquid or non-aqueous liquid, such as ethanol or glycerol; or in the form of an oil-in-water emulsion or a water-in-oil emulsion. Such oils may be edible oils, such as e.g. cottonseed oil, sesame oil, coconut oil or peanut oil. Suitable dispersing or suspending agents for aqueous suspensions include synthetic or natural gums such as tragacanth, alginate, acacia, dextran, sodium carboxymethylcellulose, gelatin, methylcellulose, hydroxypropyl methylcellulose, hydroxypropylcellulose, carbomers and polyvinylpyrrolidone. The active ingredients may also be administered in the form of a bolus, electuary or paste.

A tablet may be made by compressing or moulding the active Ingredient optionally with one or more accessory ingredients. Compressed tablets may be prepared by compressing, in a suitable machine, the active ingredient(s) in a free-flowing form such as a powder or granules, optionally mixed by a binder, such as e.g. lactose, glucose, starch, gelatine, acacia gum, tragacanth gum, sodium alginate, carboxymethylcellulose, methylcellulose, hydroxypropylmethylcellulose, polyethylene glycol, waxes or the like; a lubricant such as e.g. sodium oleate, sodium stearate, magnesium stearate, sodium benzoate, sodium acetate, sodium chloride or the like; a disintegrating agent such as e.g. starch, methylcellulose, agar, bentonite, croscarmellose sodium, sodium starch glycollate, crospovidone or the like or a dispersing agent, such as polysorbate 80. Moulded tablets may be made by moulding, in a suitable machine, a mixture of the powdered active ingredient and suitable carrier moistened with an inert liquid diluent.

Formulations for rectal administration may be in the form of suppositories in which the compound of the present invention is admixed with low melting water soluble or insoluble solids such as cocoa butter, hydrogenated vegetable oils, polyethylene glycol or fatty acids esters of polyethylene glycols, while elixirs may be prepared using myristyl palmitate.

Formulations suitable for parenteral administration conveniently comprise a sterile oily or aqueous preparation of the active ingredients, which is preferably isotonic with the blood of the recipient, e.g. isotonic saline, isotonic glucose solution or buffer solution. The formulation may be conveniently sterilised by for instance filtration through a bacteria retaining filter, addition of sterilising agent to the formulation, irradiation of the formulation or heating of the formulation. Liposomal formulations as disclosed in e.g. Encyclopedia of Pharmaceutical Technology, vol. 9, 1994, are also suitable for parenteral administration.

Alternatively, the compound of formula I may be presented as a sterile, solid preparation, e.g. a freeze-dried powder, which is readily dissolved in a sterile solvent immediately prior to use.

Transdermal formulations may be in the form of a plaster or a patch.

Formulations suitable ophthalmic administration may be in the form of a sterile aqueous preparation of the active ingredients, which may be in microcrystalline form, for example, in the form of an aqueous microcrystalline suspension. Liposomal formulations or biodegradable polymer systems e.g. as disclosed in Encyclopedia of Pharmaceutical Technology, vol. 2, 1989, may also be used to present the active ingredient for ophthalmic administration.

Formulations suitable for topical or ophthalmic administration include liquid or semi-liquid preparations such as liniments, lotions, gels, applicants, oil-in-water or water-in-oil emulsions such as creams, ointments or pastes; or solutions or suspensions such as drops.

Formulations suitable for nasal or buccal administration include powder, self-propelling and spray formulations, such as aerosols and atomisers. Such formulations are disclosed in greater detail in e.g. Modern Pharmaceutics, 2^{nd} ed., G. S. Banker and C. T. Rhodes (Eds.), page 427-432, Marcel Dekker, New York; Modern Pharmaceutics, 3^{th} ed., G. S. Banker and C. T. Rhodes (Eds.), page 618-619 and 718-721, Marcel Dekker, New York and Encyclopedia of Pharmaceutical Technology vol. 10. J Swarbrick and J. C. Boylan (Eds), page 191-221, Marcel Dekker, New York

In addition to the aforementioned ingredients, the formulations of a compound of formula I or Ia may include one or more additional ingredients such as diluents, buffers, flavouring agents, colourant, surface active agents, thickeners, preservatives, e.g. methyl hydroxybenzoate (including anti-oxidants), emulsifying agents and the like.

The parenteral formulations are in particular useful in the treatment of conditions in which a quick response to the treatment is desirable. In the continuous therapy of patients suffering from infectious diseases, the tablets or capsules may be the appropriate form of pharmaceutical preparation owing to the prolonged effect obtained when the drug is given orally, in particular in the form of sustained-release tablets.

When the active ingredient is administered In the form of salts with pharmaceutically acceptable non-toxic acids or bases, preferred salts are for instance easily water-soluble or slightly soluble in water, in order to obtain a particular and appropriate rate of absorption.

As suggested above, the composition may contain other therapeutically active components, which can appropriately be administered together with the compounds of the invention in the treatment of infectious diseases, such as other suitable antibiotics, in particular such antibiotics which may enhance the activity and/or prevent development of resistance. Corticosteroids may also beneficially be included in the compositions of the present invention. In particular, said other active component may include β-lactams, such as penicillins (phenoxymethyl penicillin, benzyl penicillin, dicloxacillin, ampicillin, amoxicillin, pivampicillin, flucloxacillin, piperacillin and mecellinam), cefalosporins (cefalexin, cefalotin, cefepim, cefotaxim, ceftazidim, ceftriazon and cefuroxim), monobactams (aztreonam) and carbapenems (meropenem); macrolides (azithromycin, clarithromycin, erythromycin and roxithromycin); polymyxins (colistin); tetracyclins (tetracycline, doxycyclin, oxytetracyclin and lymecyclin); aminoglycosides (streptomycin, gentamicin, tobramycin and netilmicin); fluoroquinolones (norfloxacin, ofloxacin, ciprofloxacin and moxifloxacin); clindamycin, lincomycin, telcoplanin, vancomycin, oxazolidones (linezolid), rifamycin, metronidazol and fusidic acid. Other compounds which advantageously may be combined with the compounds of the invention, especially for topical treatments, include e.g. corticosteroids, such as hydrocortisone, betamethason-17-valerate and triamcinolone acetonid.

The other therapeutically active compound may be in the same or separate containers adapted for concomitant or sequential administration of said therapeutically active compounds.

The treatment of infectious diseases often involves determining whether said disease is resistant or refractory to the treatment, before the treatment is, in fact, initiated. By way of example, samples containing the Infectious microbe may be taken from the patient, e.g. blood or urine, after which the sample is cultured and exposed to the treatment to determine whether said infectious organism responds to the treatment. Accordingly, the present invention also provides a method for identifying compounds effective against a microorganism, the method comprising administering a compound of formula I or Ia, optionally together with other therapeutically active agents, to a microorganism, and determining whether said compound or mixture of compounds has a toxic or static effect on the microorganism in question.

The compositions of the present invention are not limited to pharmaceuticals, but may also be used in a non-therapeutic context to control microbial growth. For example may compositions or compounds of the present invention be useful as additives which inhibit microbial growth, such as during fermentation processes. By way of example, the selectivity of antimicrobial agents renders them useful to enhance growth of particular microorganisms at the expense of others in a multi-species culture.

Biological Activity

In vitro investigations have evidenced high potency of compounds of the invention against strains of both staphylococci and streptococci which are among the most relevant pathogenic bacteria involved in various skin and eye infections. Biological tests have showed equal or in some cases slightly enhanced antibacterial activity against staphylococci of compounds of the invention compared to that of fusidic acid and, more importantly, a significantly improved antibacterial activity against streptococci as appears from Table 1 showing MIC values of selected compounds of formula Ia towards both staphylococci and streptococci.

Compounds.

The fusidic acid analogues of the invention and the reference compounds 201 (fusidic acid (as the sodium salt)), 207, 205 203 and 206 (see notes to Table A) were stored in powder form at +4° C. When used in assays, they were dissolved in 95% EtOH (3.84 mg/ml) and kept for a maximum of 1 month at −20° C. before being discarded.

Bacterial strains used for biological evaluation

| Bacterial strain | Origin |
|---|---|
| Staphylococcus aureus FDA486 | Laboratory strain |
| Staphylococcus aureus CJ12 | Laboratory strain |
| Staphylococcus aureus 8325-4 | Laboratory strain |
| Streptococcus pyogenes DA7121 | Clinical isolate from human skin |
| | infection |
| Streptococcus pyogenes DA7864 | Clinical isolate from human skin |
| | infection |

Media

LB media (per 1000 ml ddH₂O: 10 g Bacto-tryptone, 5 g yeast extract, 10 g NaCl). THB (Todd-Hewitt Broth) media, SIGMA, product number: T1438 (per 1000 ml ddH₂O: 50 g Beef-Heart Infusion, 20 g Casein peptone, 2 g Dextrose, 2 g NaHCO₃, 2.5 g NaCl, 0.4 g Na₂HPO₄). Plates were made using an agar concentration of 1.5%. Blood-agar plates contained an additional 5% (v/v) defibrinated horse blood purchased from SLU (Swedish Agricultural University), Uppsala.

MIC (Minimum Inhibitory Concentration) Determination.

MIC tests on the compounds were done in 96-well micro titer plates (Thermo Labsystems). 4×10⁵ bacteria were inoculated in 0.4 ml growth media (S. aureus, LB broth, S. pyogenes—TH broth) containing serial dilutions of the compound to be tested starting from 128 μg/ml (dilution factor 2, e.g. 128 μg/ml, 64 μg/ml, . . . , 0.016 μg/ml). The criterion for sensitivity is no visible growth after a 24 h, aerobic incubation at 37° C. Each compound was tested at least twice, and fusidic acid was always included as an experimental control.

TABLE A
Antibacterial activity measured for selected compounds
of the invention. MIC/μg · ml⁻¹.

| | Staph. | Staph. | Staph. | Strep. | Strep. |
|---|---|---|---|---|---|
| | aureus | aureus | aureus | pyogenes | pyogenes |
| Compound no. | FDA486 | CJ12 | 8325-4 | DA7121 | DA7864 |
| 108 | 0.05 | 0.03 | n.t. | 0.8 | 0.8 |
| Ref. comp. 201 | 0.11 | 0.03 | 0.03 | 3.5 | 3.5 |
| (Fusidic acid) | | | | | |
| 113 | 0.22 | 0.11 | n.t. | 0.4 | 0.4 |
| Ref. comp. 207 | 0.22 | 0.05 | n.t. | 1.6 | 1.6 |
| 115 | 0.88 | 0.06 | 0.11 | 1.8 | 1.8 |
| Ref. comp. 205 | 0.44 | 0.06 | 0.11 | 14 | 28 |
| 116 | 0.44 | 0.06 | 0.11 | 7 | 7 |
| Ref. comp. 203 | 0.22 | 0.06 | 0.22 | 7 | 14 |
| 122 | 0.88 | 0.22 | 0.88 | 7 | 7 |
| Ref. comp. 206 | 0.22 | 0.06 | 0.11 | >32 | 28 |

Notes to Table A:
Concentration of cells at t = 0: ˜10⁶/ml. Bacteria grown aerobically in broth at 37° C.
n.t. = not tested
Ref. comp. = reference compound
The reference compounds in Table A are known fusidic acid derivatives. Each reference compound refers to the compound of the invention written above in the same column. The reference compounds are unsubstituted at C-24 and have a double bond between C-24 and C-25. All other structural features of the reference compounds are identical to the corresponding compounds of the invention written above in the same column:
201 Fusidic acid
207 16-Deacetoxy-16β-isopropylsulfinyl-fusidic acid (von Daehne, W. et al., Adv. Appl. Microbiol., 1979, vol. 25, p. 95-146)
205 17S,20S-Dihydrofusidic acid (Duvold, T. et al., J. Med. Chem., 2001, Vol 44, p. 3125-3131)
203 16-Deacetoxy-16β-ethoxy-fusidic acid (von Daehne, W. et al., Adv. Appl. Microbiol., 1979, vol. 25, p. 95-146)
206 17S,20S-Methylene-fusidic acid (Duvold T., et al., Bioorg. Med. Chem. Lett., 2002, Vol. 12, p. 3569-3572)

The above data clearly show that substitution of fusidic acid at position 24 gives rise to a significant Increase in the activity against streptococci (2-15 fold) while the activity against staphylococci is essentially retained.

### ABBREVIATIONS

The following standard abbreviations are used throughout this disclosure:

AcOH=acetic acid

Ac₂O=acetic anhydride

Ac=acetyl

aq.=aqueous

Bu=n-butyl

^{t}Bu, tBu=tert-butyl

Comp.=Compound

DBU=1,8-diazabicyclo[5.4.0]undec-7-ene

DMF=dimethylformamide

eq.=equivalent

Et=ethyl

Ether=diethyl ether

EtOAc=ethyl acetate

EtOH=ethanol

Ex.=Example

FA=fusidic acid or fusidic acid analogue ring-A,B,C,D substructure

FCC=Flash Column Chromatography

Fu=fusidic acid ring-A,B,C,D substructure

HMPA=Hexamethyl phosphoric acid triamide

HPLC=High Performance Liquid Chromatography

IPr=isopropyl

Me=methyl

MeOH=methanol

m.p.=melting point

MRSA=meticilline resistant Staphylococcus aureus

Pet.ether=petroleum ether

Ph=phenyl

Phenac=phenacyl

Piv=pivaloyl

Prep.=Preparation

THF=tetrahydrofuran

TLC=Thin Layer Chromatography

rt=room temperature

sat.NaCl=saturated aqueous sodium chloride solution

TMS=trimethylsilyl

v=volume

Preparation of the Compounds of the Invention

The compounds of formula I may be synthesized from known starting materials by different synthetic routes, depending on the requirements presented by each individual compound I, such as to the availability of starting materials, the temporary protection of sensitive substituents, the purities and yields in the synthetic steps, and the selection of the preferred order of these steps.

Illustrative, but non-limiting, methods and examples of the synthesis of different compounds of formula I or Ia are given below. The various methods of synthesis may be combined with one another, as judged convenient by a person skilled in the art, to furnish the desired compounds of formula I or Ia with the desired substitution in ring A, C and D, in the 24-position in the side chain, and with regard to the free acids, salts, or the easily hydrolysable esters.

A general method for the synthesis of 24-bromo compounds of general formula I with FA (fusidic acid/fusidic acid analogue) ring-A,B,C,D substructures from starting materials 201-206 (with the same ring-A,B,C,D substructures) is shown in Scheme 1:

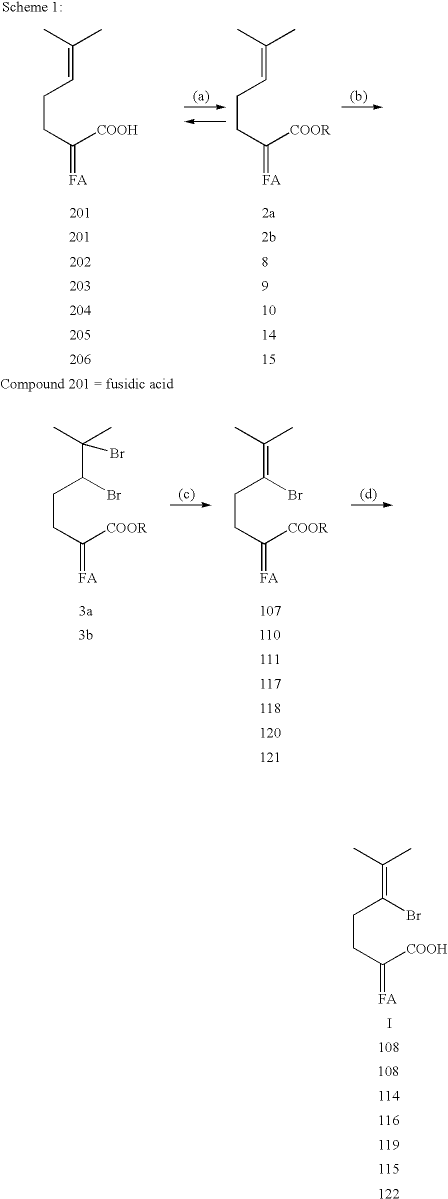

Exemplified fusidic acid and fusidic acid analogue ring-A,B,C,D substructures, FA:

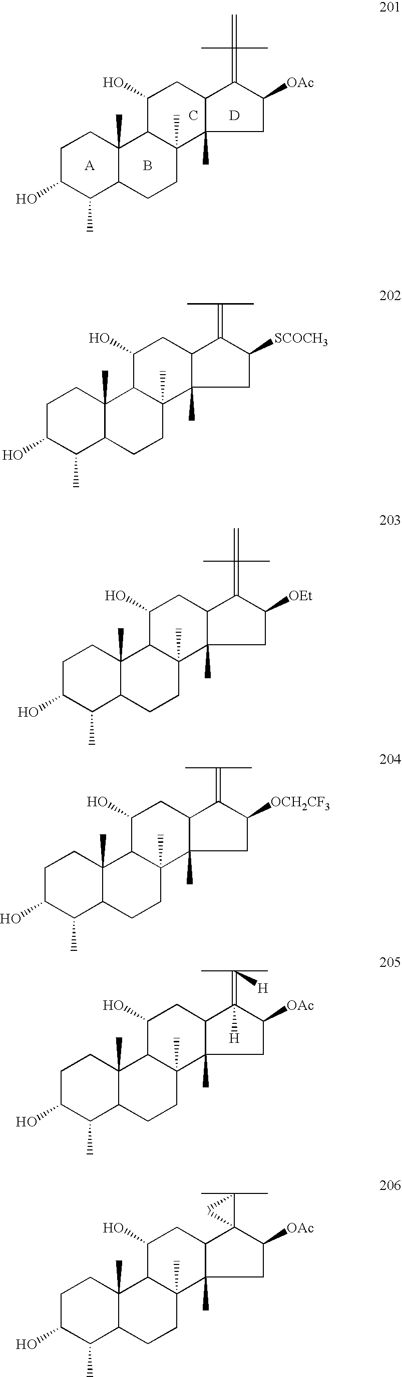

Conditions: (a) ClCH₂O(CO)R′, Et₃N, DMF (R′=Me or C(CH₃)₃), rt; (b) Br₂, CCl₄, 0° C.; (c) DBU, CCl₄ or CH₃CN, rt; (d) DBU/aq. MeOH or K₂CO₃/MeOH, rt

Fusidic acid or a fusidic acid analogue may be esterified with e.g. chloromethyl acetate or chloromethyl pivalate in a suitable solvent, such as dimethylformamide, in the presence of a suitable base, such as triethylamine. The ester can then be brominated with bromine in a suitable solvent, such as carbontetrachloride or acetonitrile. The dibromide obtained (which is usually a mixture of the 24-diastereolsomers) can be de-hydrobrominated by treatment with a suitable base, such as DBU, in a suitable solvent, such as carbontetrachloride or acetonitrile, to give mainly the 24-bromo-fusidic acid- or fusidic acid analogue-ester. If desired, the ester can be used as a prodrug of the corresponding free acid I, e.g. when having an easily hydrolysable ester group. Alternatively the ester can be hydrolyzed with a suitable base, such as DBU or K₂CO₃, in a suitable solvent, such as methanol or ethanol containing water, to give the desired compounds of general structure I (where X=bromo) as the free acids or as salts. In another embodiment of the present invention, the dibromide is de-hydrobrominated and hydrolyzed in one step to give the desired compound I as the free acid or as salts, e.g. by using methods as described above. In another preferred embodiment the fusidic acid or a fusidic acid analogue is brominated directly using methods as described above to give the corresponding dibromo acid (which is usually a mixture of the 24-diastereoisomers). The dibromo acid is then be de-hydrobrominated e.g. with methods as described above to give the desired compounds of general structure I (where X=bromo) as the free acids, optionally as salts.

A currently favoured method of preparing compounds of formula Ia wherein Q₁, Q₂, A and B are as indicated above, Y and Z together with the C-17/C-20 bond form a double bond between C-17 and C-20 or together are methylene or both represent hydrogen, and X is bromo, is illustrated by the reaction depicted in Scheme 1a (in which FA is fusidic acid or a fusidic acid analogue ring-A,B,C,D substructures as shown in Scheme 1):

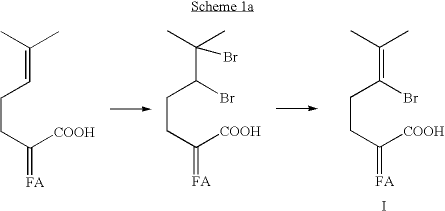

Fusidic acid or a fusidic acid analogue is dissolved in a suitable solvent, such as acetic acid or a C₁₋₄ alkyl ester of a C₁₋₄ carboxylic acid, e.g. ethyl acetate, isopropyl acetate, tert-butyl acetate, and treated with bromine, preferably dissolved in the same solvent, at −10° C.-20° C., preferably at 0° C.-10° C., such as 5° C., to give a 24,25-dibromo intermediate. The 24,25-dibromo intermediate may be isolated, optionally after addition of aqueous base and/or a reducing agent, such as Na₂SO₃, and phase separation by evaporation of the organic solvent. The 24,25-dibromo intermediate is then (optionally without isolation or purification steps) dehydrobrominated to a compound of formula Ia by reacting a solution of the 24,25-dibromo intermediate in a suitable solvent, such as a C₁₋₆ alcohol, e.g. methanol, ethanol, 1-propanol, isopropanol or butanol, or water, or mixtures of said solvents, with a suitable base at e.g. reflux temperature or for example at 50° C.-120° C., such as at 60° C.-90° C., such as 70° C.-80° C., to give the dehydrobrominated compound of formula Ia in the form of a salt. The base used to produce the dehydrobrominated compounds of formula Ia may be suitably be selected from an alkali metal or alkaline earth metal salt of a weak acid, such as carbonic, phosphoric or boric acid, e.g. potassium or sodium carbonate, or a base such as ammonia or C₁₋₈ substituted ammonia, e.g. ethylamine, diethylamine, triethylamine or piperidine, or an alkali or alkaline earth metal hydroxide such as dilute sodium hydroxide, calcium hydroxide or dilute potassium hydroxide. The compound of formula Ia in free acid form may then be obtained from the salt by acidification with a suitable acid, such as aqueous phosphoric acid or acetic acid. Compound Ia may then be either purified and recrystallized, e.g. as described in example 8 and 45 below, to give the pure compound Ia, or converted into an easily hydrolysable ester, e.g. by using the procedure described in preparations 1 and 2, or converted into a suitable salt, such as a sodium salt, e.g. as described in example 9 below.

A General method for the conversion of the 24-bromo substituent of a compound of general formula I (X=bromo) into 24-substituted compounds of general formula I (X≠bromo) as illustrated by conversion of compound 108 is shown in Scheme 2:

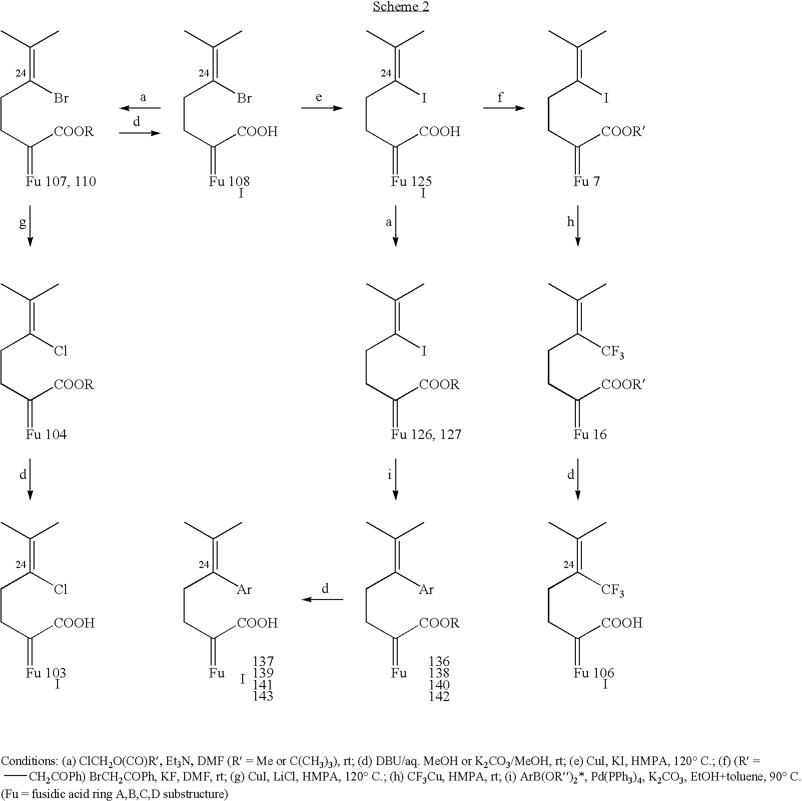

The 24-bromo-fusidic acid- or 24-bromo-fusidic acid analogue-acetoxymethyl esters or -pivaloyloxymethylesters may be hydrolyzed to the corresponding free acids e.g. by treatment with methanol and aqueous base. The bromo-acids may be heated with copper (I) iodide and potassium iodide in HMPA at 120° C., to give the corresponding 24-iodo acid of formula I. The acids can be esterified to the corresponding phenacyl esters by treatment with phenacylbromide and potassium fluoride in DMF. The phenacyl esters yield the corresponding 24-trifluoromethyl esters e.g. upon reaction with a solution of trifluoromethyl copper in HMPA. The esters may finally be converted to the free 24-trifluoromethyl fusidic acids (or fusidic acid analogues) of formula I upon hydrolysis, such as alkaline hydrolysis.

Alternatively, the 24-iodo acids can be esterified to their acetoxymethyl esters or pivaloyloxymethylesters as described above, and they can be converted to the corresponding 24-aryl, or alkenyl esters etc., by suitable coupling reactions, e.g. with a Suzuki-type coupling with an aryl boronic acid, or ester, or substituted aryl boronic acid, or ester, as shown in Scheme 2. Finally, the corresponding free acids of compounds of general formula I may be obtained by hydrolysis of the ester, such as alkaline hydrolysis.

In another embodiment the 24-bromo-fusidic acid-, or 24-bromo-fusidic acid analogue-acetoxymethyl esters, or pivaloyloxymethylesters, may be heated with copper (I) iodide and lithium chloride in HMPA, to give the corresponding 24-chloro ester. This ester gives the free 24-chloro acids of compounds of general formula I after hydrolysis, such as alkaline hydrolysis.

A general method for the synthesis of compounds I, comprising modifications in ring A during the synthetic sequence, illustrated for the synthesis of compounds 123, 124 and 144, is shown in Scheme 3:

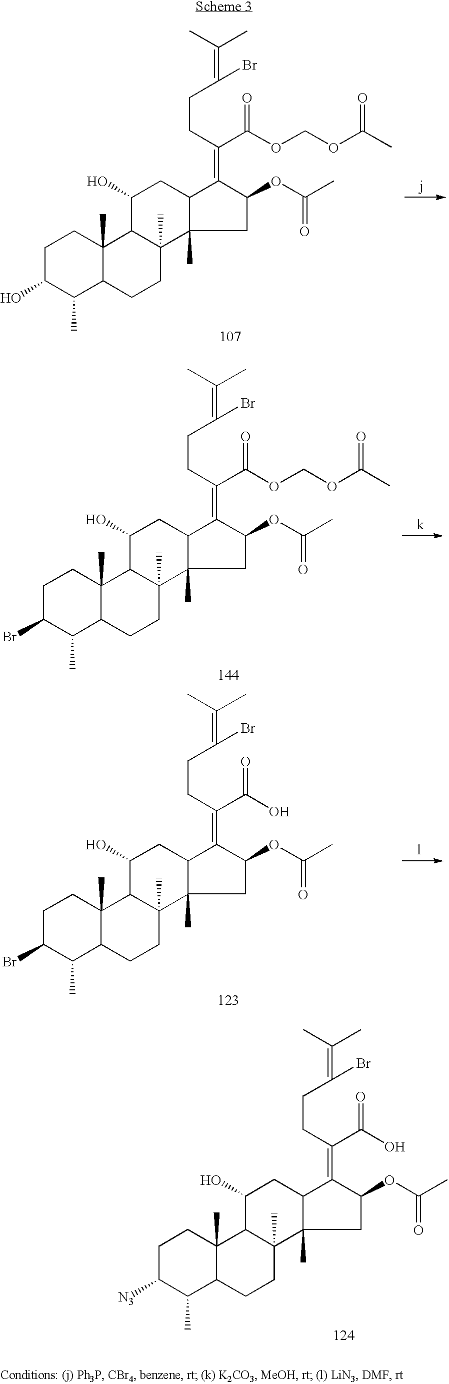

A non-limiting example of modification of the substitution in one of the rings of the fusidic acid ring-A,B,C,D substructure, e.g. after the 24 substituent has been introduced, is illustrated in Scheme 3 for modifications in ring A: For example, the 24-bromo-fusidic acid-, or 24-bromo-fusidic acid analogue-acetoxymethyl ester, or -pivaloyloxymethylester, can be brominated with triphenylphosphine and tetrabromomethane to give, with inversion of configuration, the corresponding 3-β-bromo ester. The ester can optionally be hydrolyzed to the free acid of formula I. This acid can be further modified, e.g. as shown, by treatment with lithium azide, to give, with another inversion of configuration, the corresponding 3-α-azido ester of formula I.

A general method for the synthesis of compounds I, comprising modifications in ring A and ring D during the synthetic sequence, illustrated for the synthesis of compounds 112 and 113 is shown in Scheme 4:

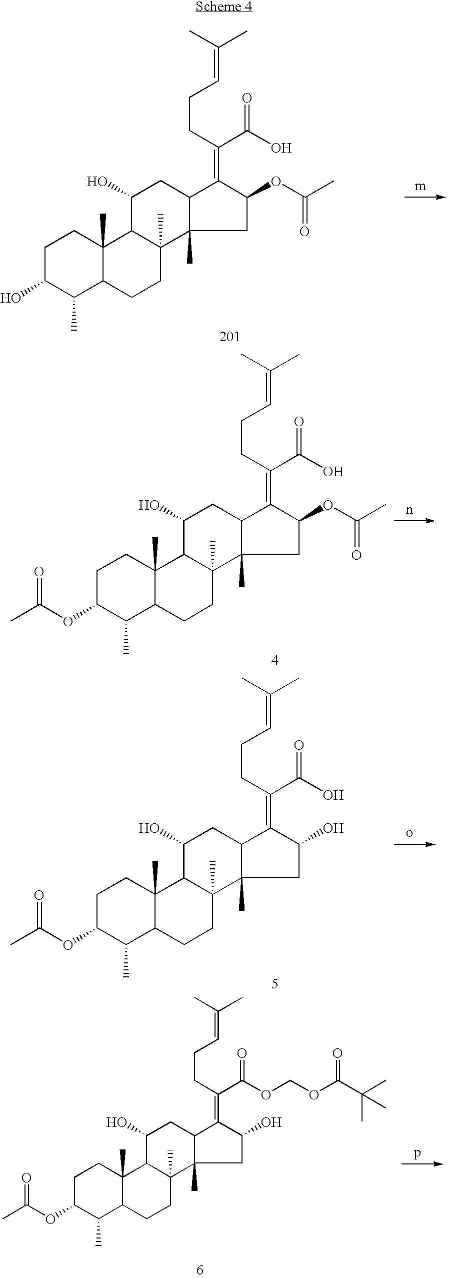

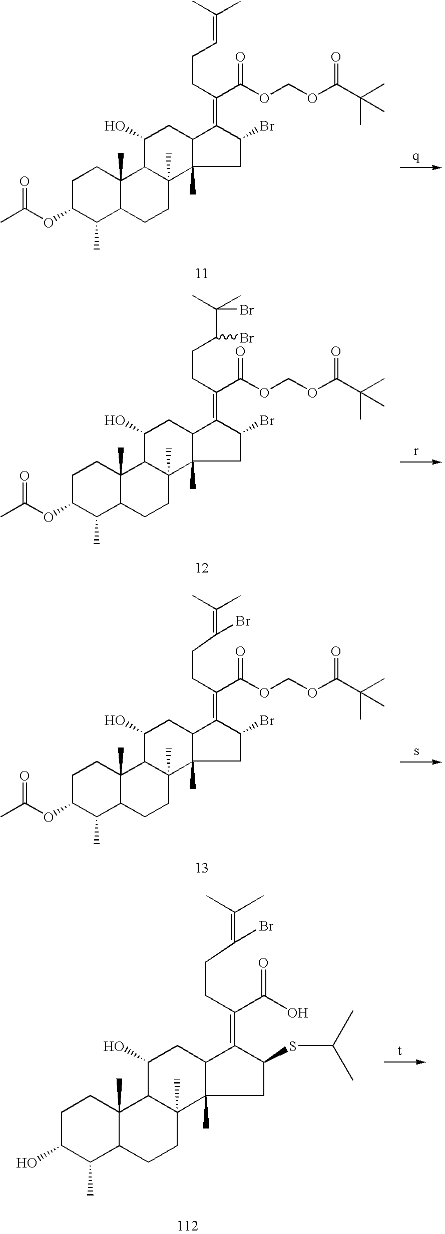

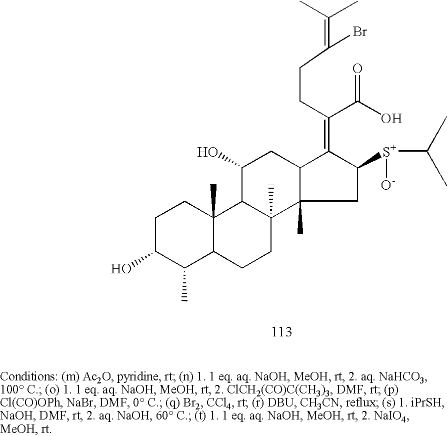

The synthesis of compounds 112 and 113 starting with fusidic acid, illustrates a non-limiting procedure in which the 16-substituent in ring D may be changed to an alkylthio- or alkylsulfinyl-group with 16-β-stereochemistry during the synthesis of compounds of general formula I. Temporary protection of the 3-hydroxy group and the carboxy group may advantageously be applied, and bromine in position 24 may be introduced at an appropriate stage In the synthetic sequence: Fusidic acid (201) can be acetylated at C3 with acetic anhydride and pyridine to give compound (4). The corresponding sodium salt of compound (4) can then be heated with aq. sodium hydrogen carbonate yielding the 16-α-hydroxy compound (5) (with inversion of configuration at C16). The sodium salt of (5) can for example be esterified with chloromethyl pivalate to give (6). Compound (6) can be treated with phenyl chloroformate, dimethylformamide and sodium bromide to give the 16-α-bromo compound (11) (with retention of configuration at C16). Compound (11) can be brominated to give the 24,25-dibromo compound (12), which can be dehydrobrominated with e.g. DBU to the 24-bromo compound (13). Alkylation of sodium isopropylthiolate with compound (13) gives the 16-β-isopropylthio intermediate (with inversion of configuration at C16) which may be hydrolyzed with e.g. aq. base to the 24-bromo-3-α-hydroxy-16-β-isopropylthio carboxylic acid (112) of formula I. If desired, compound (112) can be oxidized (with e.g. sodium periodate) to the corresponding sulfoxide (113).

### PREPARATIONS AND EXAMPLES

General

All melting points are uncorrected. For ¹H (300 MHz) and ¹³C (75.6 MHz) nuclear magnetic resonance (NMR) spectra chemical shift values (δ) (in ppm) are quoted, unless otherwise specified, for deuteriochloroform solutions relative to Internal tetramethylsilane (δ=0.00) or deuteriochloroform (δ=76.81 for ¹³C NMR). The value for a multiplet, either defined (doublet (d), triplet (t), quartet (q)) or not (m) at the approximate mid point is given unless a range is quoted (s=singlet, b=broad). Reaction mixtures were usually worked up by: extraction with an (indicated) organic solvent, which was shaken with water and/or aq. solutions of (indicated) salts or acids; the organic solutions were usually dried over sodium or magnesium sulfate, and concentrated under reduced pressure on a rotary evaporator. Chromatography was performed on silica gel usually using ethyl acetate and low boiling petroleum ether as eluant. The solvent ratios used are indicated as volume ratios/percent (v:v). The appropriate fractions were combined and concentrated, in some cases followed by crystallisation or freeze-drying. Solvents: anhydrous solvents were normally prepared by storing analytical grade solvents over 4 Å molecular sieves a few days prior to use.

Preparation of Intermediates for the Synthesis of Compounds I

The intermediates of general formula Ib are listed In Table 1:

TABLE 1

| | |
|---|---|
| | [Ib] |
| 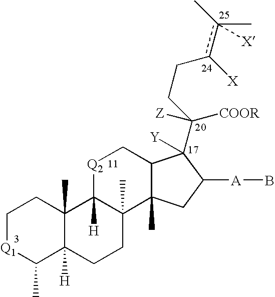 | |

Exemplified intermediates of general formula Ib

| Prep. | Comp. | Comm. | | | | | |
|---|---|---|---|---|---|---|---|
| No. | No. | Proced. | Q₁ | A-B | Y, Z | R | X, (X′) |
| 1 | 2a | Prep.1 | CH—OH (α) | O—Ac (β) | Bd | CH₂OAc | H |
| 2 | 2b | Prep.2 | CH—OH (α) | O—Ac (β) | Bd | CH₂OAc | H |
| 3 | 3a | Prep.3 | CH—OH (α) | O—Ac (β) | Bd | CH₂OAc | Br, Br |
| 4 | 3b | Prep.3 | CH—OH (α) | O—Ac (β) | Bd | CH₂OPiv | Br, Br |
| 5 | 4 | | CH—OAc (α) | O—Ac (β) | Bd | H | H |
| 6 | 5 | | CH—OAc (α) | O—H (α) | Bd | H | H |
| 7 | 6 | | CH—OAc (α) | O—H (α) | Bd | CH₂OPiv | H |
| 8 | 8 | Prep.1 | CH—OH (α) | S—Ac (β) | Bd | CH₂OAc | H |
| 9 | 9 | Prep.1 | CH—OH (α) | OEt (β) | Bd | CH₂OAc | H |
| 10 | 10 | Prep.1 | CH—OH (α) | OCH₂CF₃ (β) | Bd | CH₂OAc | H |
| 11 | 11 | | CH—OAc (α) | Br (α) | Bd | CH₂OPiv | H |
| 12 | 12 | Prep.3 | CH—OAc (α) | Br (α) | Bd | CH₂OPiv | Br, Br |
| 13 | 13 | | CH—OAc (α) | Br (α) | Bd | CH₂OPiv | Br |
| 14 | 14 | Prep.1 | CH—OH (α) | O—Ac (β) | H,H | CH₂OAc | H |
| 15 | 15 | Prep.1 | CH—OH (α) | O—Ac (β) | —CH₂— | CH₂OAc | H |
| 16 | 7 | | CH—OH (α) | O—Ac (β) | Bd | phenac | I |
| 17 | 16 | | CH—OH (α) | O—Ac (β) | Bd | phenac | CF₃ |
| 18 | 17 | | CH—OH (α) | O—Ac (β) | Bd | H | Br, Br |

Notes to formula Ib and Table 1:
Prep. = Preparation; Prep. = The procedure is used in other preparations or examples;
Comp. = Compound; Comm.Proced. = Common Procedure; Q₂ = CH—OH (α);
X For those compounds which do not contain X′, the configuration around C#24 and C#25 is the same as in formula Ia, i.e. C24 and C25 are connected by a double bond. For those compounds where X = X′ = Br, both C24 and C25 are substituted with a bromine atom, and C24 and C25 are connected by a single bond and the compounds are mixtures of the two C24 diastereoisomers.
R H = the free acid; Na = the sodium salt; CH₂OAc = the acetoxymethyl ester
CH₂OPiv = the pivaloyloxymethyl ester; phenac = the phenacyl ester
Y, Z Bd = carbon-carbon bond, i.e. C17 and C20 are connected by a double bond;
H, H = 17S-H, 20S-H, i.e. C17 and C20 are connected by a single bond; —CH₂— = [Y, Z]-C17-C20 forms a cyclopropane ring with 17S, 20S-stereochemistry.

Preparations

Preparation 1: Fusidic Acid Acetoxymethyl Ester (2a)

To a solution of fusidic acid (201) (128.6 g; 250 mmol) in DMF (375 ml) Et₃N (45 ml; 33 g; 320 mmol) was added and the mixture was stirred for 30 minutes at rt. Chloromethyl acetate (49 ml; 55 g; 500 mmol) was now added and the reaction mixture was stirred overnight at rt and then worked up (EtOAc, water) to give a crude product. The crude ester (2a) was crystallized from isopropylether to afford pure compound (2a) as a colourless powder, m.p. 103-105° C. ¹³C NMR, (CDCl₃): 170.4, 169.6, 168.4, 150.6, 132.7, 129.3, 122.9, 79.4, 74.4, 71.4, 68.2, 49.2, 48.7, 44.3, 39.5, 39.0, 37.1, 36.2, 36.2, 35.5, 32.4, 30.3, 30.0, 28.8, 28.3, 25.7, 24.2, 22.8, 20.9, 20.8, 20.7, 17.9, 17.7, 15.9

Preparation 2: Fusidic Acid Pivaloyloxymethyl Ester (2b)

By following the procedure given for preparation 1 and replacing chloromethyl acetate with chloromethyl pivalate, and carrying out the reaction at 50° C. overnight, fusidic acid pivaloyloxymethyl ester (2b) was obtained as a colourless, amorphous powder. ¹³C NMR, (CDCl₃): 177.0, 170.2, 168.1, 150.9, 132.6, 129.3, 123.0, 79.8, 74.3, 71.4, 68.2, 49.3, 48.8, 44.3, 39.5, 39.0, 38.8, 37.0, 36.3, 36.1, 35.6, 32.3, 30.2, 30.0, 28.8, 28.3, 26.9, 25.7, 24.1, 22.9, 20.8, 17.9, 17.8, 15.9.

Preparation 3: 24R,S,25-Dibromofusidic Acid Acetoxymethyl Ester (3a)

Fusidic acid acetoxymethyl ester (2a) (6 g; 10 mmol) was dissolved in CCl₄ (40 ml) and a solution of bromine (0.56 ml; 1.76 g; 11 mmol) in CCl₄ (40 ml) was added in the course of one hour with continuous stirring and cooling in an ice bath. The resulting, slightly yellow, solution was used in the following step without further purification. ¹H NMR, (CDCl₃): 5.91 (m, 1H), 5.78 (bs, 2H), 4.36 (bs, 1H), 4.20 (m, 1H), 3.75 (bs, 1H), 3.16 (m, 1H), 2.80-1.00 (m, 20H), 2.10 (s, 3H), 1.97 (bs, 6H), 1.80 (s, 3H), 1.38 (s, 3H), 0.96 (s, 3H), 0.95 (s, 3H), 0.91 (d, 3H).

Preparation 4: 24R,S,25-Dibromofusidic Acid Pivaloyloxymethyl Ester (3b)

By following the procedure given for preparation 3 and replacing fusidic acid acetoxymethyl ester (2a) with fusidic acid pivaloyloxymethyl ester (2b), and after concentrating the reaction mixture, purifying the crude product by means of FCC (hexane : EtOAc 50:50 as eluant), the title compound 3b was obtained as a colourless foam. ¹³C NMR, (CDCl₃): 177.0, 170.2, 170.2, 167.7, 167.6, 153.0, 153.0, 127.7, 80.1, 80.0, 74.3, 71.4, 68.5, 68.4, 68.2, 68.1, 66.2, 65.8, 60.4, 49.3, 49.2, 48.9, 48.9, 44.5, 39.5, 39.0, 38.8, 37.0, 36.3, 36.1, 35.8, 35.2, 35.1, 32.3, 31.6, 30.2, 30.0, 28.5, 28.2, 28.2, 27.7, 26.9, 24.1, 24.1, 22.8, 22.7, 20.8, 20.8, 18.1, 18.0, 16.0, 14.2.

Preparation 5: 3-Acetyl-Fusidic Acid (4)

Fusidic acid (201) (74.3 g; 0.144 mol) was dissolved in pyridine (75 ml; 74 g; 0.93 mol) and acetic anhydride (75 ml; 81 g; 0.79 mol) and the resulting reaction mixture was stirred at rt for three hours, after which the reaction was complete. The acetylated product was precipitated by addition of ice and water. Recrystallization from methanol/water yielded the pure compound (4). ¹³C NMR, (CDCl₃):174.5, 171.0, 170.6, 151.1, 132.7, 129.7, 123.0, 74.4, 74.2, 68.3, 49.1, 48.8, 44.3, 39.4, 39.0, 37.8, 37.0, 35.8, 34.8, 32.7, 31.1, 28.7, 28.4, 27.4, 25.7, 24.4, 22.6, 21.3, 20.6, 20.6, 18.1, 17.8, 15.5.

Preparation 6: 3-Acetyl-16-Deacetoxy-16α-Hydroxy Fusidic Acid (5)

3-Acetoxy-fusidic acid (4) (9.95 g; 17.8 mmol) was dissolved in MeOH (250 ml) and neutralized with an equivalent amount of aqueous NaOH (about 9 ml, 2M). The solvents were evaporated and water (150 ml) was added to the residue. The mixture was heated to reflux and 20 ml of a saturated aqueous solution of NaHCO₃ (ca. 1 M) was added over a period of 30 minutes. The resulting clear solution was heated to 100° C. for eight hours after which an insoluble by-product (the corresponding lactone) was formed. The lactone was removed by filtration, and the filtrate was acidified with HCl (20 ml, 4M) and extracted with EtOAc. The organic phase was washed with water, dried with MgSO₄, and concentrated under reduced pressure to give the title compound (5) which was used in the following step without further purification. ¹³C NMR, (CDCl₃): 174.2, 171.2, 164.7, 132.5, 127.6, 123.2, 74.2, 72.2, 68.4, 49.1, 47.4, 43.9, 39.5, 39.2, 37.7, 36.9, 35.9, 34.9, 32.6, 31.0, 29.1, 28.4, 27.3, 25.7, 24.5, 22.7, 21.4, 20.7, 18.4, 17.9, 15.5.

Preparation 7: 3-Acetyl-16-deacetoxy-16α-hydroxy fusidic acid pivaloyloxymethyl ester (6)

3-Acetyl-16-deacetoxy-16α-hydroxy fusidic acid (5) (39.7 g; 77 mmol) was dissolved in MeOH (250 ml) and neutralized with 1 eq. of aq. NaOH. The solvent was evaporated and the residue redissolved in DMF (450 ml). Chloromethylpivalate (13.4 ml; 13.9 g; 92 mmol) was added over a period of 30 minutes with continuous stirring and ice-cooling. The resulting mixture was stirred overnight at rt, after which it was worked up (EtOAc, aq. CaCl₂, water, sat. NaCl), dried with MgSO₄, and concentrated under reduced pressure to give the title compound (6) as an oil which was used in the next step without further purification (preparation 11). ¹³C NMR, (CDCl₃): 177.2, 171.0, 168.6, 164.6, 132.6, 127.2, 123.1, 80.0, 74.1, 72.1, 68.4, 49.1, 47.4, 43.7, 39.5, 39.4, 38.8, 37.7, 36.9, 36.0, 34.9, 32.6, 31.0, 28.9, 28.0, 27.4, 26.9, 25.7, 24.5, 22.6, 21.3, 20.7, 18.4, 17.8, 15.5.

Preparation 8: 16-Deacetoxy-16β-Thioacetyl-Fusidic Acid Acetoxymethylester (8)

By following the procedure given for preparation (1) and replacing fusidic acid with 16-deacetoxy-16β-thioacetyl-fusidic acid (202) (von Daehne, W. et al., Adv. App. Microbiol., 1979, vol. 25, p. 95-146), and using 10 eq. each of Et₃N and chloromethyl acetate, and purifying the crude product by FCC with pet.ether:EtOAc 1:1 as eluant, the title compound (8) was obtained. ¹³C NMR, (CDCl₃): 194.9, 169.5, 168.4, 151.3, 132.7, 129.5, 122.9, 80.0, 71.4, 68.3, 49.2, 49.0, 45.7, 43.7, 41.4, 39.7, 37.2, 36.3, 35.9, 35.7, 32.7, 30.4, 30.0, 29.9, 29.3, 28.3, 25.7, 24.4, 22.4, 20.7, 20.6, 18.6, 17.7, 16.0.

Preparation 9: 16-Deacetoxy-16β-Ethoxy-Fusidic Acid Acetoxymethylester (9)

By following the procedure given for preparation 1 and replacing fusidic acid with the potassium salt of 16-deacetoxy-16β-ethoxy-fusidic acid (203) (von Daehne, W. et al., Adv. Appl. Microbiol., 1979, vol. 25, p. 95-146) and using no Et₃N and 10 eq. of chloromethyl acetate, and purifying the crude product by FCC with pet.ether:EtOAc 1:1 as eluant, the title compound (9) was obtained. ¹³C NMR, (CDCl₃): 169.7, 169.6, 151.2, 132.4, 128.6, 123.2, 79.6, 78.8, 71.4, 68.4, 65.2, 49.2, 49.0, 43.3, 39.5, 37.0, 36.3, 36.2, 35.8, 35.5, 32.5, 30.2, 30.0, 28.8, 28.2, 25.7, 24.1, 22.8, 20.9, 20.8, 17.8, 17.7, 16.0, 15.3.

Preparation 10: 16-Deacetoxy-16β-(2′,2′,2′-trifluoroethoxy) -Fusidic Acid Acetoxymethylester (10)

By following the procedure given for preparation (1) and replacing fusidic acid with 16-deacetoxy-16β-(2′,2′,2′-trifluoroethoxy)-fusidic acid (204) (von Daehne, W et al., Adv. Appl. Microbiol., 1979, vol. 25, p. 95-146) and using 10 eq. each of Et₃N and chloromethyl acetate, and purifying the crude product by FCC with pet.ether:EtOAc 1:1 as eluant, the title compound (10) was obtained. ¹³C NMR, (CDCl₃): 169.7, 169.1, 151.0, 132.6, 129.9, 123.7, 123.0, 80.1, 79.5, 71.4, 68.3, 67.8, 49.1, 49.0, 43.8, 39.5, 37.1, 36.3, 36.2, 35.8, 35.5, 32.6, 30.3, 30.0, 28.6, 28.2, 25.7, 24.3, 22.7, 20.8, 20.7, 17.7, 17.6, 15.9.

Preparation 11: 3-Acetyl-16α-Bromo-16-Deacetoxy-Fusidic Acid Pivaloyloxymethyl Ester (11)

3-Acetyl-16-deacetoxy-16α-hydroxy fusidic acid pivaloyloxymethyl ester (6) (22.8 g; 36.2 mmol) was dissolved in DMF (200 ml) and cooled in an ice bath under an atmosphere of argon and with continuous stirring. Sodium bromide (18.6 g; 181 mmol) was added to the solution and the resulting mixture was stirred for one hour. Phenyl chloroformate (22.8 ml; 28.3 g; 181 mmol) was added over a period of one hour at 0° C., followed by stirring for 18 hours at rt. The reaction-mixture was worked up (EtOAc, aq. CaCl₂, water, sat.NaCI), dried with MgSO₄, and concentrated under reduced pressure to yield a crude product. The crude product was purified by FCC (10% to 30% EtOAc in pet.ether as eluant) to yield the pure title compound (11) as an oil. ¹³CNMR,(CDCl₃): 177.3, 171.0, 167.5, 154.8, 132.6, 129.5, 123.0, 79.8, 74.1, 68.2, 50.6, 49.3, 48.8, 43.5, 42.0, 39.5, 38.9, 37.6, 36.9, 35.8, 35.0, 32.5, 30.9, 28.6, 28.3, 27.3, 27.0, 25.7,24. 3,22.8, 21.3, 20.7, 17.8, 17.4, 15.5.

Preparation 12: 3-Acetyl-16-Deacetoxy-16α-24,25-Tribromo Fusidic Acid Pivaloyloxymethyl Ester (12)

By following the procedure given for preparation 3 and replacing fusidic acid acetoxymethyl ester (2a) with 3-Acetyl-16α-bromo-16-deacetoxy-fusidic acid pivaloyloxymethyl ester (11), the title compound (12) was obtained as a colourless foam. ¹H NMR, (CDCl₃): 5.87 (m, 2H), 5.64 (bt, 1H), 4.93 (bs, 1H), 4.35 (bs, 1H), 4.14 (dd, 1H), 3.46 (bd, 1H), 2.80-1.00 (m, 20H), 2.07 (s, 3H), 1.97 (s, 3H), 1.84 (s, 3H), 1.49 (s, 3H), 1.22 (s, 9H), 0.98 (s, 3H), 0.83 (d, 3H), 0.78 (s, 3H).

Preparation 13: 3-Acetyl-16-Deacetoxy-16α, 24-Dibromo Fusidic Acid Pivaloyloxymethyl Ester (13)

3-Acetyl-16-deacetoxy-16α-24,25-tribromo fusidic acid pivaloyloxymethyl ester (12) (14.4 g; 16.4 mmol) and DBU (7.4 ml; 7.6 g; 49 mmol) were dissolved in acetonitrile (200 ml) and the resulting solution was heated for five hours at 50° C. under an atmosphere of argon and with continuous stirring. The reaction mixture was concentrated under reduced pressure and worked up (EtOAc, water, sat.NaCl). The crude product was purified by FCC (10% to 15% EtOAc in petr.ether as eluant) to yield the title compound (13) as a crystalline product. ¹H NMR, (CDCl₃): 5.87 (d, 1H), 5.84 (d, 1H), 5.64 (bt, 1H), 4.94 (bs, 1H), 4.36 (bs, 1H), 3.45 (bd, 1H), 2.75-2.50 (m,5H), 2.30-1.00 (m, 15H), 2.07 (s, 3H), 1.85 (s, 3H), 1.78 (s, 3H), 1.46 (s, 3H), 1.23 (s, 9H), 0.98 (s, 3H), 0.83 (dd, 3H), 0.77 (s, 3H).

Preparation 14: 17S,20S-Dihydrofusidic Acid Acetoxymethylester (14)

By following the procedure given for preparation (1) and replacing fusidic acid with 17S,20S-dihydrofusidic acid (205) (Duvold, T. et al., J. Med. Chem., 2001, Vol 44, p. 3125-3131) and using 10 eq. each of Et₃N and chloromethyl acetate, and purifying the crude product by FCC with pet.ether:EtOAc 1:1 as eluant, the title compound (14) was obtained. ¹³C NMR, (CDCl₃): 173.8, 170.0, 169.8, 132.4, 123.3, 78.7, 76.5, 71.4, 68.8, 49.3, 45.7, 44.1, 40.6, 38.3, 37.1, 36.3, 34.3, 32.7, 32.5, 30.3, 30.0, 25.7, 25.2, 23.7, 22.8, 21.0, 20.9, 20.7, 17.7, 17.2, 16.0.

Preparation 15: 17S,20S-Methylene-Fusidic Acid Acetoxymethylester (15)

By following the procedure given for preparation (1) and replacing fusidic acid with 17S,20S-methylene-fusidic acid (206) (Duvold T., et al., Bioorg. Med. Chem. Lett., 2002, Vol. 12, p. 3569-3572) and using 10 eq. each of Et₃N and chloromethyl acetate, and purifying the crude product by FCC with pet.ether:EtOAc 1:1 as eluant, the title compound (15) was obtained. ¹³C NMR, (CDCl₃): 171.5, 170.1, 169.6, 132.2, 123.6, 79.1, 78.8, 71.4, 68.3, 49.7, 48.5, 42.6, 40.1, 39.9, 38.6, 37.1, 36.4, 36.3, 36.1, 34.6, 32.3, 31.8, 30.3, 29.9, 26.0, 25.7, 24.1, 22.9, 20.7, 20.7, 18.9, 18.0, 17.6, 16.0.

Preparation 16: 24-Iodo-Fusidic Acid Phenacylester (7)

A mixture of phenacylbromide (0.42 g; 2.1 mmol), potassium fluoride (0.27 g; 4.6 mmol) and DMF (10 ml) was stirred for five minutes at 90° C. under an atmosphere of argon. 24-Iodo-fusidic acid (125,) (1.35 g; 2.1 mmol) was added, and the resulting mixture was stirred for one hour at 90° C. The reaction was worked up (ether, water, sat. NaCl, MgSO₄) and concentrated under reduced pressure to yield the title compound (7) as an amorphous powder. ¹³C NMR, (CDCl₃): 171.1, 170.5, 168.6, 152.3, 137.4, 134.3, 133.8, 128.9, 128.2, 127.8, 100.3, 74.4, 71.4, 68.2, 65.8, 60.4, 49.3, 48.9, 44.7, 41.6, 39.5, 39.1, 37.0, 36.4, 36.1, 36.0, 32.2, 31.7, 30.2, 30.0, 28.9, 24.0, 22.9, 21.0, 20.9, 19.4, 18.0, 16.0, 14.2.

Preparation 17: 24-Trifluoromethyl-Fusidic Acid Phenacylester (16)

A solution of trifluoromethyl copper complex in HMPA (Kobayashi, Y. et al., Tetrahedron. Lett., 1979, Vol. 42, p. 4071-4072), made from trifluoromethyl iodide (0.43 g, 2.2 mmol) and copper powder (0.32 g, 5 mgAt) in HMPA (1.5 ml), was added to 24-iodo-fusidic acid phenacylester (7) (190 mg, 0.25 mmol). The resulting mixture was stirred in a closed vial for 3 days at rt, under an atmosphere of argon, then worked up with EtOAc, water and sat.NaCI, dried and concentrated under reduced pressure. The crude product was purified by FCC (20% to 40% EtOAc In pet.ether as eluant), followed by preparative HPLC (Lichrospher®-100 RP18, with a gradient of 50% to 0% 0.01 M aq. NH₄⁺HCOO⁻ mixed with 0.01 M NH₄⁺HCOO⁻ in 9:1 acetonitrile:water as eluant). The appropriate fractions were combined, concentrated under reduced pressure and extracted with EtOAc; concentration of the EtOAc solution under reduced pressure gave the title compound (16) as an oil. ¹H NMR, (CDCl₃): 7.88 (dd, 2H), 7.58 (t, 1H), 7.48 (t, 2H), 5.98 (d, 1H), 5.48 (d, 1H), 5.11 (d, 1H), 4.36 (s, 1H), 3.75 (bs, 1H), 3.10 (bd, 1H), 2.75-1.00 (m, 21H), 2.01 (s, 3H), 1.88 (,3H), 1.83 (,3H), 1.38 (s, 3H), 0.98 (s, 3H), 0.93 (s, 3H), 0.92 (d, 3H).

Preparation 18: 24,25-Dibromo-Fusidic Acid (17)

A solution of bromine (16.0 g, 0.1 mol) in ethyl acetate (100 ml) was added to a stirred solution of fusidic acid (51.6 g, 0.1 mol) in ethyl acetate (1000 ml), over a period of 75 minutes. The temperature was kept at 5° C. by cooling in an ice bath. KH₂PO₄ (100 ml, 1M aq.) and Na₂S₂O₃ (50 ml, 1M aq.) were added, during a few minutes. The EtOAc-phase was separated and extracted with KH₂PO₄ (200 ml, 0.5M aq.) and water (100 ml), then concentrated under reduced pressure to give a solid residue of (17) which was used without further purification in the following step (Example 45). 1H NMR (CDCl₃): 5.81 (d, 1H), 4.32 (m, 1H), 4.26 (t, 1H), 3.66 (m, 1H), 3.09 (m, 1H), 3.0-1.0 (m, 19H), 1.82 (s, 3H), 1.81 (s, 3H), 1.39 (s, 3H), 1.00 (s, 3H), 0.99 (s, 3H), 0.94 (s, 3H), 0.89 (d, 3H).

Compounds I of the Invention

Exemplified compounds of general formula I are listed in Table 2 (for compounds of general formula Ia where Q₂=CH—OH (α)):

TABLE 2

[Ia]

Exemplified compounds of general formula Ia (Q₂ = CH—OH (α))

| Ex. | Comp. | Comm. | | | | |
|---|---|---|---|---|---|---|
| No. | No. | Proced. | Q₁ | A-B | Y, Z | X |
| 3 | 103 | Ex.3 | CH—OH (α) | O—Ac | Bd | Cl |
| 6 | 106 | | CH—OH (α) | O—Ac | Bd | CF₃ |
| 8 | 108 | | CH—OH (α) | O—Ac | Bd | Br |
| 12 | 112 | Ex.12 | CH—OH (α) | 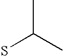 | Bd | Br |
| 13 | 113 | Ex.13 | CH—OH (α) | 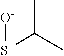 | Bd | Br |
| 14 | 114 | Ex.14 | CH—OH (α) | S—Ac | Bd | Br |
| 15 | 115 | Ex.14 | CH—OH (α) | O—Ac | H,H | Br |
| 16 | 116 | Ex.14 | CH—OH (α) | O—Et | Bd | Br |
| 19 | 119 | Ex.14 | CH—OH (α) | O—CH₂CF₃ | Bd | Br |
| 22 | 122 | Ex.14 | CH—OH (α) | O—Ac | —CH₂— | Br |
| 23 | 123 | Ex.14 | CH—Br (α) | O—Ac | Bd | Br |
| 24 | 124 | | CH—N₃ (α) | O—Ac | Bd | Br |
| 25 | 125 | Ex.25 | CH—OH (α) | O—Ac | Bd | I |
| 37 | 137 | Ex.3 | CH—OH (α) | O—Ac | Bd | Ph |
| 39 | 139 | Ex.3 | CH—OH (α) | O—Ac | Bd | 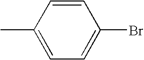 |
| 41 | 141 | Ex.3 | CH—OH (α) | O—Ac | Bd | 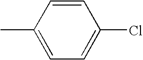 |
| 43 | 143 | Ex.3 | CH—OH (α) | O—Ac | Bd | 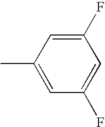 |
| 45 | 108 | | CH—OH (α) | O—Ac | Bd | Br |
| 46 | 146 | Ex.12 | CH—OH (α) | 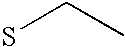 | Bd | Br |
| 47 | 147 | Ex.13 | CH—OH (α) | 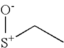 | Bd | Br |
| 48 | 148 | Ex.12 | CH—OH (α) | 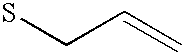 | Bd | Br |
| 49 | 149 | Ex.12 | CH—OH (α) | 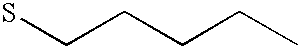 | Bd | Br |
| 50 | 150 | Ex.13 | CH—OH (α) | 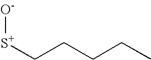 | Bd | Br |
| 51 | 151 | Ex.12 | CH—OH (α) | 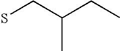 | Bd | Br |
| 52 | 152 | Ex.13 | CH—OH (α) | 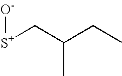 | Bd | Br |
| 53 | 153 | Ex.12 | CH—OH (α) | 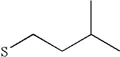 | Bd | Br |
| 54 | 154 | Ex.13 | CH—OH (α) | 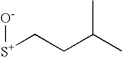 | Bd | Br |
| 55 | 155 | Ex.55 | CH—OH (α) | 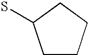 | Bd | Br |
| 56 | 156 | Ex.12 | CH—OH (α) | 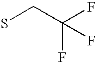 | Bd | Br |
| 57 | 157 | Ex.55 | CH—OH (α) | 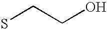 | Bd | Br |
| 58 | 158 | Ex.12 | CH—OH (α) | 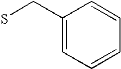 | Bd | Br |
| 59 | 159 | Ex.13 | CH—OH (α) | 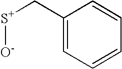 | Bd | Br |
| 60 | 160 | Ex.12 | CH—OH (α) | 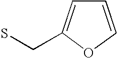 | Bd | Br |
| 61 | 161 | Ex.12 | CH—OH (α) | 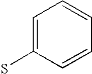 | Bd | Br |
| 62 | 162 | Ex.55 | CH—OH (α) | 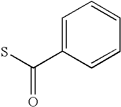 | Bd | Br |
| 63 | 163 | Ex.55 | CH—OH (α) | 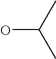 | Bd | Br |
| 64 | 164 | Ex.55 | CH—OH (α) | 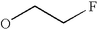 | Bd | Br |
| 65 | 165 | Ex.55 | CH—OH (α) | 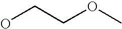 | Bd | Br |
| 66 | 166 | Ex.66 | CH—OH (α) | OAc | Bd | 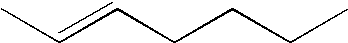 |
| 67 | 167 | Ex.66 | CH—OH (α) | OAc | Bd | 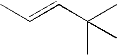 |
| 68 | 168 | Ex.66 | CH—OH (α) | OAc | Bd | 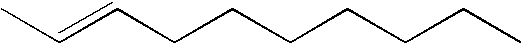 |
| 69 | 169 | Ex.66 | CH—OH (α) | OAc | Bd | 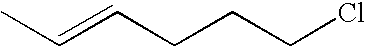 |
| 70 | 170 | Ex.66 | CH—OH (α) | OAc | Bd | 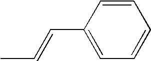 |
| 71 | 171 | | CH—OH (α) | OAc | Bd | 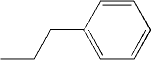 |
| 72 | 172 | Ex.66 | CH—OH (α) | OAc | Bd | 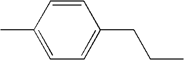 |
| 73 | 173 | Ex.66 | CH—OH (α) | OAc | Bd | 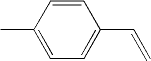 |
| 74 | 174 | Ex.66 | CH—OH (α) | OAc | Bd | 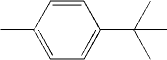 |
| 75 | 175 | Ex.66 | CH—OH (α) | OAc | Bd | 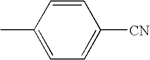 |
| 76 | 176 | Ex.66 | CH—OH (α) | OAc | Bd | 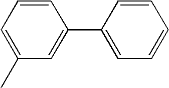 |
| 77 | 177 | Ex.66 | CH—OH (α) | OAc | Bd | 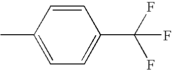 |
| 78 | 178 | Ex.66 | CH—OH (α) | OAc | Bd | 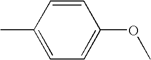 |
| 79 | 179 | Ex.66 | CH—OH (α) | OAc | Bd | 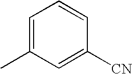 |
| 80 | 180 | Ex.66 | CH—OH (α) | OAc | Bd | 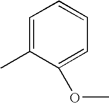 |
| 81 | 181 | Ex.66 | CH—OH (α) | OAc | Bd | 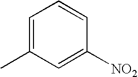 |
| 82 | 182 | Ex.66 | CH—OH (α) | OAc | Bd | 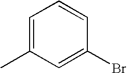 |
| 83 | 183 | Ex.66 | CH—OH (α) | OAc | Bd | 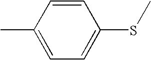 |
| 84 | 184 | Ex.66 | CH—OH (α) | OAc | Bd | 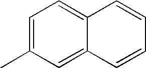 |
| 85 | 185 | Ex.66 | CH—OH (α) | OAc | Bd | 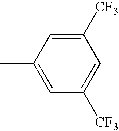 |
| 86 | 186 | Ex.66 | CH—OH (α) | OAc | Bd | 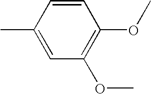 |
| 87 | 187 | Ex.66 | CH—OH (α) | OAc | Bd | 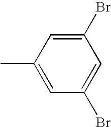 |

Notes to table 2:
Symbols of Table 2 which are common with those of Table 1, have the same meanings. Ex. = Example; Ex. = The procedure is used in other examples.

Exemplified easily hydrolysable esters of compounds of general formula I are listed in Table 3a (for acetoxymethyl esters) and Table 3b (for pivaloyloxymethylesters) (for esters of compounds of general formula Ia (Q₂=CH—OH (α)):

TABLE 3a
Exemplified acetoxymethyl esters of compounds of
general formula Ia (Q₂ = CH—OH (α))

| Ex. | Comp. | Comm. | | | | |
|---|---|---|---|---|---|---|
| No. | No. | Proced. | Q₁ | A-B | Y, Z | X |
| 7 | 107 | Ex. 7 | CH—OH (α) | O—Ac | Bd | Br |
| 11 | 111 | Ex. 11 | CH—OH (α) | S—Ac | Bd | Br |
| 17 | 117 | Ex. 11 | CH—OH (α) | O-Et | Bd | Br |
| 18 | 118 | Ex. 11 | CH—OH (α) | O—CH₂CF₃ | Bd | Br |
| 20 | 120 | Ex. 11 | CH—OH (α) | O—Ac | H, H | Br |
| 21 | 121 | Ex. 11 | CH—OH (α) | O—Ac | —CH₂— | Br |
| 26 | 126 | Ex. 25 | CH—OH (α) | O—Ac | Bd | I |
| 44 | 144 | Ex. 3 | CH—Br (β) | O—Ac | Bd | Br |

TABLE 3b
Exemplified pivaloyloxymethylesters esters of compounds of general formula Ia (Q₂ = CH—OH (α))

| Ex. | Comp. | Comm. | | | | |
|---|---|---|---|---|---|---|
| No. | No. | Proced. | Q₁ | A—B | Y,Z | X |
| 2 | 102 | Prep. 2 | CH—OH (α) | O—Ac | Bd | CF₃ |
| 4 | 104 | | CH—OH (α) | O—Ac | Bd | Cl |
| 10 | 110 | Ex. 7 | CH—OH (α) | O—Ac | Bd | Br |
| 27 | 127 | | CH—OH (α) | O—Ac | Bd | I |
| 36 | 136 | Ex. 36 | CH—OH (α) | O—Ac | Bd | Ph |
| 38 | 138 | Ex. 36 | CH—OH (α) | O—Ac | Bd | 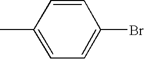 |
| 40 | 140 | Ex. 36 | CH—OH (α) | O—Ac | Bd | 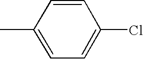 |
| 42 | 142 | Ex. 36 | CH—OH (α) | O—Ac | Bd | 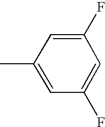 |
| 106 | 306 | Ex. 36 | CH—OH (α) | O—Ac | Bd | 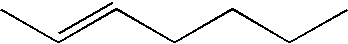 |
| 107 | 307 | Ex. 36 | CH—OH (a) | O—Ac | Bd | 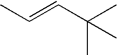 |
| 108 | 308 | Ex. 36 | CH—OH (α) | O—Ac | Bd | 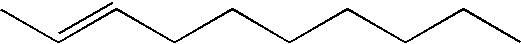 |
| 109 | 309 | Ex. 36 | CH—OH (α) | O—Ac | Bd | 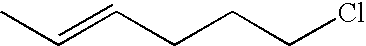 |
| 110 | 310 | Ex. 36 | CH—OH (α) | O—Ac | Bd | 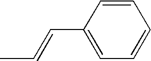 |
| 111 | 311 | | CH—OH (α) | O—Ac | Bd | 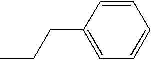 |
| 112 | 312 | Ex. 36 | CH—OH (α) | O—Ac | Bd | 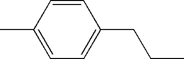 |
| 113 | 313 | Ex. 36 | CH—OH (α) | O—Ac | Bd | 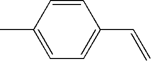 |
| 114 | 314 | Ex. 36 | CH—OH (α) | O—Ac | Bd | 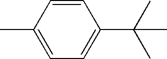 |
| 115 | 315 | Ex. 36 | CH—OH (α) | O—Ac | Bd | 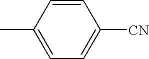 |
| 116 | 316 | Ex. 36 | CH—OH (α) | O—Ac | Bd | 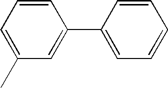 |
| 117 | 317 | Ex. 36 | CH—OH (α) | O—Ac | Bd | 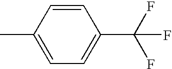 |
| 118 | 318 | Ex. 36 | CH—OH (α) | O—Ac | Bd | 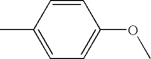 |
| 119 | 319 | Ex. 36 | CH—OH (α) | O—Ac | Bd | 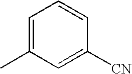 |
| 120 | 320 | Ex. 36 | CH—OH (α) | O—Ac | Bd | 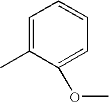 |
| 121 | 321 | Ex. 36 | CH—OH (α) | O—Ac | Bd | 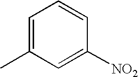 |
| 122 | 322 | Ex. 36 | CH—OH (α) | O—Ac | Bd | 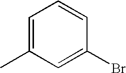 |
| 123 | 323 | Ex. 36 | CH—OH (α) | O—Ac | Bd | 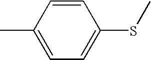 |
| 124 | 324 | Ex. 36 | CH—OH (α) | O—Ac | Bd | 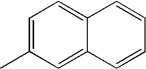 |
| 125 | 325 | Ex. 36 | CH—OH (α) | O—Ac | Bd | 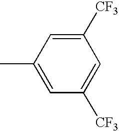 |
| 126 | 326 | Ex. 36 | CH—OH (α) | O—Ac | Bd | 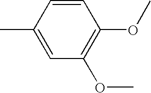 |
| 127 | 327 | Ex. 36 | CH—OH (α) | O—Ac | Bd | 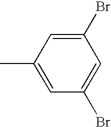 |

Notes to table 3a and 3b:
Symbols of Table 3a and 3b which are common with those of Table 1, have the same meanings.
Ex. = Example;
Ex. = The procedure is used in other examples.

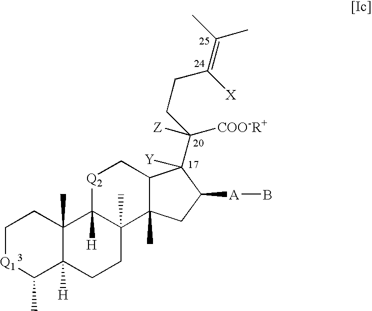

Exemplified salts of compounds of general formula I are listed in Table 4 (for salts of general formula Ic (Q₂=CH—OH (α)):

TABLE 4
Exemplified salts of compounds of general formula Ic (Q₂ = CH—OH (α))

| Ex. | Comp. | Comm. | | | | | |
|---|---|---|---|---|---|---|---|
| No. | No. | Proced. | Q₁ | A-B | Y, Z | R⁺ | X |
| 1 | 101 | Ex.9 | CH—OH (α) | O—Ac | Bd | Na⁺ | CF₃ |
| 5 | 105 | Ex.9 | CH—OH (α) | O—Ac | Bd | Na⁺ | Cl |
| 9 | 109 | Ex.9 | CH—OH (α) | O—Ac | Bd | Na⁺ | Br |
| 88 | 188 | Ex.88 | CH—OH (α) | O—Ac | Bd | 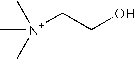 | Br |
| 89 | 189 | Ex.88 | CH—OH (α) | O—Ac | Bd | 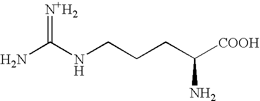 | Br |
| 90 | 190 | Ex.88 | CH—OH (α) | O—Ac | Bd | 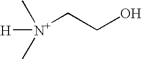 | Br |
| 91 | 191 | Ex.88 | CH—OH (α) | O—Ac | Bd | 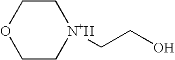 | Br |
| 92 | 192 | Ex.88 | CH—OH (α) | O—Ac | Bd | 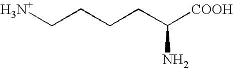 | Br |
| 93 | 193 | Ex.88 | CH—OH (α) | O—Ac | Bd | 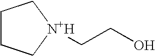 | Br |
| 94 | 194 | Ex.88 | CH—OH (α) | O—Ac | Bd | 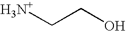 | Br |
| 95 | 195 | Ex.95 | CH—OH (α) | O—Ac | Bd | K⁺ | Br |
| 96 | 196 | Ex.95 | CH—OH (α) | O—Ac | Bd | N⁺(n-C₄H₉)₄ | Br |
| 97 | 197 | Ex.95 | CH—OH (α) | O—Ac | Bd | 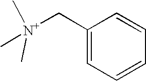 | Br |
| 98 | 198 | Ex.95 | CH—OH (α) | O—Ac | Bd | (CH₃)₃N⁺(CH₂)₁₅CH₃ | Br |
| 99 | 199 | Ex.95 | CH—OH (α) | O—Ac | Bd | N⁺(CH₃)₄ | Br |
| 100 | 300 | Ex.95 | CH—OH (α) | O—Ac | Bd | N⁺(n-C₃H₇)₄ | Br |
| 101 | 301 | Ex.88 | CH—OH (α) | O—Ac | Bd | H₃N⁺C(CH₂OH)₃ | Br |
| 102 | 302 | Ex.88 | CH—OH (α) | O—Ac | Bd | 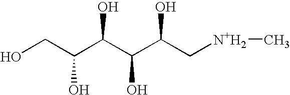 | Br |
| 103 | 303 | | CH—OH (α) | O—Ac | Bd | Ag⁺ | Br |
| 104 | 304 | | CH—OH (α) | O—Ac | Bd | 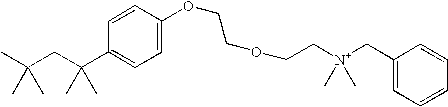 | Br |
| 105 | 305 | Ex.88 | CH—OH (α) | O—Ac | Bd | HN⁺(CH₂CH₂OH)₃ | Br |

Notes to table 4:
Symbols of Table 3a and 3b which are common with those of Table 1, have the same meanings. Ex. = Example; Ex. = The procedure is used in other examples.

### EXAMPLES

### Example 1

### 24-Trifluoromethyl-fusidic acid sodium salt (Compound 101

The title compound (101) can be obtained by following the procedure of example 9 and replacing 24-Bromo-fusidic acid (108) with 24-trifluoromethyl fusidic acid (106).

### Example 2

### 24-Trifluoromethyl-fusidic acid pivaloyloxymethyl ester (Compound 102)

The title compound (102) can be obtained by following the procedure of preparation 2 and replacing fusidic acid with 24-trifluoromethyl fusidic acid (106), and freeze-drying the product.

### Example 3

### 24—Chloro-fusidic acid (Compound 103)

24—Chloro-fusidic acid pivaloyloxymethyl ester (104) (140 mg, 0.21 mmol) and K₂CO₃ (60 mg, 0.43 mmol) were stirred in MeOH (2 ml) for 3 hours at rt. By FCC of the concentrated reaction mixture, (pet.ether:EtOAc:HCOOH, 90:10:1 to 10:90:1 as eluant), pure title compound 103 was obtained. ¹³C NMR, (CDCl₃): 174.0, 170.6, 152.5, 128.5, 126.8, 74.5, 71.5, 68.2, 49.3, 48.8, 44.6, 39.5, 39.0, 37.0, 36.3, 36.0, 35.6, 32.2, 30.2, 29.9, 27.3, 24.0, 22.9, 21.9, 20.8, 20.6, 20.3, 17.9, 15.9.

### Example 4

### 24-Chloro-fusidic acid pivaloyloxymethyl ester (Compound 104)

24-Bromo-fusidic acid pivaloyloxymethyl ester (10) (283 mg, 0.40 mmol), CuI (240 mg, 1.26 mmol), LiCl (30 mg, 0.7 mmol) and HMPA (1.2 ml) were shaken in a closed vial for 3 hours at 120° C. The reaction mixture was worked up (EtOAc and sat.NaCl) to give a crude product. This was purified by FCC with pet.ether: EtOAc (90:10 to 10:90) as eluant to give the pure title compound 104. ¹³C NMR, (CDCl₃): 177.0, 170.2, 167.8, 152.8, 128.5, 128.0, 126.7, 80.0, 74.4, 71.4, 68.2, 49.3, 48.8, 44.6, 39.5, 39.0, 38.8, 37.0, 36.4, 36.0, 35.5, 35.4, 32.2, 30.2, 29.9, 27.2, 26.9, 24.0, 22.9, 21.9, 20.8, 20.4, 17.9, 16.0, 14.2.

### Example 5

### 24-Chloro-fusidic acid sodium salt (Compound 105)

By following the procedure of example 9 and replacing 24-Bromo-fusidic acid (108) with 24-chlorofusidic acid (103), and freeze-drying the product, the title compound (105) was obtained.

### Example 6

### 24-Trifluoromethyl fusidic acid (Compound 106)

24-trifluoromethyl-fusidic acid phenacylester (17) (15 mg, 0.021 mmol) and sodium thiophenolate (20 mg, 0.15 mmol) in dry DMF (0.5 ml) was stirred under argon at 100° C. for five hours. EtOAc (15 ml) was added and the organic solution was extracted with: 3M aq. CaCl₂ (10 ml)+1M aq.H₃PO₄ (0.25 ml), and with (10 ml of each) 3M aq. CaCl₂, water and sat.NaCl. After drying and concentration the crude product was purified by FCC, with pet.ether:EtOAc:HCOOH (60:40:½) as eluant to give, after freeze-drying, the title compound 106 as an amorphous powder. ¹H NMR, (CDCl₃): 5.87 (d, 1H), 4.34 (s, 1H), 3.75 (s, 1H), 3.06 (bd, 1H), 2.70-0.80 (m, 22H), 1.98 (s, 3H), 1.85 (q, 3H), 1.83 (q, 3H), 1.37 (m, 3H), 0.97 (s, 3H), 0.91 (s, 3H), 0.90 (d, 3H).

### Example 7

### 24-Bromo-fusidic acid acetoxymethyl ester (Compound 107)

24-R,S,25-Dibromofusidic acid acetoxymethyl ester (3a) (from 22.3 mmol fusidic acid acetoxymethyl ester) in CCl₄ (280 ml) and DBU (6.64 ml; 6.77 g; 44.5 mmol) was refluxed for 16 hours. The reaction mixture was filtered from a clay-like precipitate through a cotton wool filter, and the filter was washed with pet.ether and EtOAc, The combined filtrate and washings were concentrated to give the title compound 107 as a crude product (about 70% pure, by NMR) which can be used without further purification in the preparation of compound 108 (Example 8). A pure sample was obtained by means of FCC (30% to 50% EtOAc in petroleum ether as eluant). ¹³C NMR, (CDCl₃): 170.3, 169.6, 167.9, 152.6, 131.6, 127.9, 120.0,79.5, 74.4, 71.4, 68.2, 49.3, 48.8, 44.6, 39.5, 39.0, 37.8, 37.0, 36.3, 36.0, 35.6, 32.2, 30.2, 29.9, 27.8, 25.3, 24.0, 22.9, 20.8, 20.7, 20.4, 18.0, 16.0.

### Example 8

### 24-Bromo-fusidic acid (Compound 108)

Crude 24-bromo-fusidic acid acetoxymethyl ester (107) or pivaloyloxymethylester (110) (from 44.4 mmol fusidic acid acetoxymethyl ester (2a) or pivaloyloxymethylester (2b)) was dissolved in MeOH (250 ml) and DBU (3 ml) was added to secure a basic reaction. MeOH:water 1:1 (300 ml) was added at rt, during two hours, with stirring which was continued for a further two hours. A 1M KH₂PO₄-solution was added (100 ml) if necessary also phosphoric acid, to give a pH about 4-5; the precipitate which formed was dissolved by extraction, twice, with EtOAc. The organic phase was extracted with water and sat.NaCl, dried with MgSO₄, and concentrated to give a crude product. This was purified by FCC (50% EtOAc in pet.ether+0.5% HCOOH as eluant), followed by recrystallization from EtOAc+toluene (with partial evaporation) to give the pure title compound (108). ¹³C NMR, (CDCl₃): 173.0, 170.5, 152.6, 131.5, 128.1, 120.1, 74.5, 71.4, 68.2, 49.2, 48.8, 44.6, 39.5, 39.0, 37.8, 37.1, 36.2, 36.2, 35.7, 32.4, 30.2, 29.9, 28.0, 25.3, 24.1, 22.8, 20.8, 20.7, 20.4, 18.0, 15.9.

### Example 9

### 24-Bromo-fusidic acid sodium salt (Compound 109)

24-Bromofusidic acid (108) (2.38g; 4.00 mmol) was dissolved in MeOH (30 ml). An equivalent amount of 1N NaOH-solution (4 ml) was added gradually until the pH was about 8.5, as measured with a pH-meter. The resulting solution was concentrated and the residue was dissolved in EtOH (15 ml). EtOAc (25 ml) was added, but crystallization did not take place until the solvents were evaporated, EtOH and EtOAc added and the solvents evaporated once again. The residue now crystallized from EtOH+EtOAc to give the title compound (109) as colourless crystals. ¹³C NMR, (CDCl₃): 179.1, 173.5, 138.8, 138.2, 131.3, 122.6, 76.0, 72.5, 68.9, 50.8, 50.0, 43.8, 40.7, 40.3, 38.5, 38.3, 37.8, 37.5, 36.9, 33.0, 31.1, 31.0, 30.2, 25.4, 23.8, 23.8, 22.5, 21.1, 20.5, 17.9, 16.5.

### Example 10

### 24-Bromo-fusidic acid pivaloyloxymethyl ester (Compound 110)

By following the procedure given Example 7 and replacing 24R,S,25-dibromofusidic acid acetoxymethyl ester with 24R,S,25-Dibromofusidic acid pivaloyloxymethyl ester (3b) crude title compound (110) was obtained. This can be hydrolyzed, without further purification, to give compound 108. FCC of a sample of the crude product, using 30% to 50% EtOAc in pet.ether as eluant, gave the pure title compound (110) as a light-yellow amorphous foam. ¹³C NMR, (CDCl₃): 177.0, 170.2, 167.8, 152.8, 131.5, 127.9, 120.1, 80.0, 74.4, 71.4, 68.2, 49.3, 48.8, 44.6, 39.5, 39.0, 38.8, 37.7, 37.0, 36.3, 36.0, 35.7, 32.3, 30.2, 30.0, 27.8, 26.9, 25.3, 24.0, 22.9, 20.8, 20.4, 18.0, 16.0.

### Example 11

### 24-Bromo-16-deacetoxy-16β-thioacetyl-fusidic acid acetoxymethylester (Compound 111)

A solution of bromine (45 μl; 140 mg; 0.88 mmol) in CCl₄ (5 ml) was added to a solution of 16-deacetoxy-16β-thioacetyl-fusidic acid acetoxymethylester (8) (0.48 g; 0.8 mmol) in CCl₄ (10 ml), during two hours, under argon, with stirring and cooling in an ice bath. Stirring was continued for 15 min. in the ice-bath and for a further 15 min. at rt. DBU (0.66 ml; 0.67 g; 4.4 mmol) was added, and the mixture was boiled under reflux for 12 hours. The reaction mixture was filtered through filter aid and concentrated in vacuo. The residue was purified by FCC, with 0% to 70% EtOAc in pet.ether as eluant, to give the title compound (111). ¹³C NMR, (CDCl₃): 194.8, 169.5, 168.0, 153.0, 131.7, 128.1, 120.0, 79.9, 71.4, 68.2, 49.3, 49.0, 45.9, 43.8, 41.4, 39.7, 37.6, 37.2, 36.4, 35.9, 35.8, 32.8, 30.4, 30.1, 29.9, 28.3, 25.3, 24.5, 22.4, 20.7, 20.6, 20.4, 18.8, 16.0.

### Example 12

### 24-Bromo-16-deacetoxy-16β-isopropylthio-fusidic acid (Compound 112)

2-Propanethiol (1.4 ml; 1.13 g; 15 mmol) was dissolved in dry DMF (12.5 ml) and sodium hydride (60% dispersion in oil; 0.6 g; ca. 15 mmol) was added, followed by 3-Acetyl-16-deacetoxy-16α, 24-dibromo fusidic acid pivaloyloxymethyl ester (13) (0.45 g; 0.6 mmol), with stirring at rt, and under argon. Stirring was continued for two hours and the reaction mixture was worked up (EtOAc, water, aq. HCl (to ca. pH4), water, sat.NaCl) and concentrated to an oil. This was dissolved in EtOH (20 ml), and 2 N aq. NaOH (10 ml) was added, and the mixture was heated to 60° C. for two hours. The hydrolysis-mixture was worked up as above, and the crude product was purified by FCC (10% to 20% EtOAc in petr.ether+1% AcOH, as eluant) to give the title compound (112). ¹H NMR, (CDCl₃): 4.30 (m, 1H), 4.15 (m, 1H), 3.75 (m, 1H), 3.10 (m, 1H), 1.82 (s, 3H), 1.75 (s, 3H), 1.35 (s, 3H), 1.24 (d, 3H, 3=6 Hz), 1.18 (d, 3H, J=6 Hz), 0.99 (s, 3H), 0.98 (s, 3H), 0.88 (d, 3H, 1=6 Hz) , 2.9-1.0 (m, 23H).

### Example 13

### 24-Bromo-16-deacetoxy-16β-isopropylsulfinyl-fusidic acid (Compound 113)

To a solution of 24-Bromo-16-deacetoxy-16β-isopropylthio-fusidic acid (112) (0.29 g; 0.47 mmol) in MeOH (10 ml) was added 2N aq. NaOH (0.5 ml) and sodium periodate (0.23 g; 1.1 mmol) in water (40 ml). The mixture was stirred for one hour at rt, and acidified with aq. HCl to precipitate the acid. This was filtered off, washed with water and recrystallized from EtOAc to give the title compound (113) as crystals, m.p. 166-168° C.

¹³C NMR, (CDCl₃): 173.7, 159.6, 131.3, 125.8, 120.2, 71.5, 68.3, 60.2, 51.8, 49.5, 48.2, 47.5, 39.7, 38.2, 37.2, 36.3, 36.1, 35.6, 32.6, 30.4, 30.0, 28.0 ,26.6, 25.3, 24.6, 22.7, 20.7, 20.4, 18.3, 17.8, 16.0, 13.5.

### Example 14

### 24-Bromo-16-deacetoxy-16β-thioacetyl-fusidic acid (Compound 114)

24-Bromo-16-deacetoxy-16β-thioacetyl-fusidic acid acetoxymethylester (111) (40 mg; 0.059 mmol) was dissolved in MeOH (2.5 ml) and potassium carbonate (17 mg; 0.12 mmol) was added, and the mixture was stirred for three hours (with access to the moisture of the air). Water (10 ml) was added, and the mixture was acidified to ca. pH 4 with aq. HCl by which the acid precipitated. The mixture was worked up with EtOAc, water and sat.NaCl, dried with Na₂SO₄ and concentrated to give a crude product. This was purified by FCC (0% to 10% MeOH in dichloromethane as eluant) to give the title compound (114). ¹³C NMR, (CDCl₃): 202.7, 175.6, 133.0, 131.6, 120.4, 71.4, 68.0, 54.5, 50.4, 48.5, 40.8, 40.6, 37.1, 37.0, 36.7, 36.0, 35.2, 32.7, 31.7, 30.2, 29.9, 25.3, 23.4, 23.3, 21.0, 20.4, 19.5, 16.0.

### Example 15

### 24-Bromo-17S,20S-dihydrofusidic acid (Compound 115)

By following the procedure given in Example 14 and replacing 24-Bromo-16-deacetoxy-16β-thioacetyl-fusidic acid acetoxymethylester (111) with 24-Bromo-17S,20S-dihydro-fusidic acid acetoxymethyl ester (compound 120), the title compound (115) was obtained. ¹³C NMR, (CDCl₃): 180.7, 170.1, 130.9, 120.5, 76.3, 71.5, 68.8, 49.4, 49.4, 44.9, 44.2, 40.6, 38.3, 37.2, 36.4, 36.2, 35.1, 34.3, 32.5, 31.3, 30.3, 29.9, 25.4, 23.8, 22.8, 21.0, 20.8, 20.3, 17.2, 15.9.

### Example 16

### 24-Bromo-16-deacetoxy-16β-ethoxy-fusidic acid (Compound 116)

By following the procedure given in Example 14 and replacing 24-Bromo-16-deacetoxy-16β-thioacetyl-fusidic acid acetoxymethylester (111) with 24-Bromo-16-deacetoxy-16β-ethoxy-fusidic acid acetoxymethyl ester (compound 117) the title compound (116) was obtained. ¹³C NMR, (CDCl₃): 171.0, 151.7, 132.6, 131.6, 120.2, 80.9 ,71.5, 68.4, 64.8, 49.5, 49.0, 44.2, 39.8, 37.6, 37.2, 36.5, 35.9, 35.9, 35.1, 32.8, 30.4, 30.1, 28.9, 25.3, 24.4, 22.3, 20.6, 20.5, 18.6, 16.0, 14.7.

### Example 17

### 24-Bromo-16-deacetoxy-16β-ethoxy-fusidic acid acetoxymethyl ester (Compound 117)

By following the procedure given in Example 11 and replacing 16-deacetoxy-16β-thioacetyl-fusidic acid acetoxymethylester (8) with 16-deacetoxy-16β-ethoxy-fusidic acid acetoxymethyl ester (9) the title compound (117) was obtained. ¹³C NMR, (CDCl₃): 169.7, 152.8, 131.2, 127.3, 120.4, 79.6, 78.8, 71.4, 68.4, 65.3, 49.2, 49.1, 43.4, 39.5, 37.7, 37.1, 36.3, 36.3, 35.8, 35.5, 32.5, 30.3, 30.0, 27.8, 25.3, 24.2, 22.8, 20.8, 20.9, 20.3, 17.9, 16.0, 15.3.

### Example 18

### 24-Bromo-16-deacetoxy-16β-(2′,2′,2′-trifluoroethoxy)-fusidic acid acetoxymethyl ester (Compound 118)

By following the procedure given in Example 11 and replacing 16-deacetoxy-16β-thioacetyl-fusidic acid acetoxymethylester (8) with 16-deacetoxy-16β-(2′,2′,2′-trifluoroethoxy)-fusidic acid acetoxymethyl ester (10) the title compound (118) was obtained. ¹³C NMR, (CDCl₃): 169.6, 168.7, 152.6, 131.4, 128.5, 123.8, 120.2, 80.1, 79.4, 71.4, 68.2, 67.8, 67.6, 49.1, 49.1, 44.0, 39.5, 37.6, 37.1, 36.2, 35.8, 35.6, 32.5, 30.3, 30.0, 27.6, 25.3, 24.2, 22.8, 20.7, 20.8, 20.3, 17.7, 16.0.

### Example 19

### 24-Bromo-16-deacetoxy-16β-(2′,2′,2′-trifluoroethoxy)-fusidic acid (Compound 119)

By following the procedure given In Example 14 and replacing 24-Bromo-16-deacetoxy-16β-thioacetyl-fusidic acid acetoxymethylester (111) with 24-Bromo-16-deacetoxy-16β-(2′,2′,2′-trifluoroethoxy) fusidic acid acetoxymethyl ester (118) the title compound (119) was obtained. ¹³C NMR, (CDCl₃): 175.3, 151.9, 131.4, 129.0, 123.6, 120.2, 80.5, 77.2, 71.5, 68.3, 67.8, 49.1, 49.0, 43.9, 39.6, 37.7, 37.1, 36.2, 35.8, 35.6, 32.5, 30.3, 30.0, 28.0, 25.3, 24.2, 22.8, 20.8, 20.2, 17.7, 16.0.

### Example 20

### 24-Bromo-17S,20S-dihydro-fusidic acid acetoxymethyl ester (Compound 120)

By following the procedure given in Example 11 and replacing 16-deacetoxy-16β-thioacetyl-fusidic acid acetoxymethylester (8) with 17S,20S-dihydro-fusidic acid acetoxymethyl ester (14) the title compound (120) was obtained. ¹³C NMR, (CDCl₃): 173.6, 169.9, 169.7, 130.8, 120.4, 78.8, 76.5, 71.4, 68.8, 49.4, 49.3, 45.4, 43.9, 40.6, 40.6, 38.3, 37.2, 36.4, 36.2, 35.0, 34.2, 32.6, 31.2, 30.4, 30.0, 25.3, 23.8, 22.7, 20.9, 20.9, 20.7, 20.3, 17.2, 16.0.

### Example 21

### 24-Bromo-17S,20S-methylene-fusidic acid acetoxymethyl ester (Compound 121)

By following the procedure given in Example 11 and replacing 16-deacetoxy-16β-thioacetyl-fusidic acid acetoxymethylester (8) with 17S,20S-methylene-fusidic acid acetoxymethyl ester (15) the title compound (121) was obtained. ¹³C NMR, (CDCl₃): 171.5, 169.9, 169.6, 130.9, 120.9, 79.2, 79.0, 71.4, 68.3, 49.6, 48.5, 43.3, 40.4, 39.9, 39.2, 37.1, 36.3, 36.2, 35.9, 35.7, 34.6, 32.4, 30.3, 30.0, 29.5, 25.4, 24.2, 22.8, 21.1, 20.9, 20.7, 20.2, 19.4, 17.9, 15.9.

### Example 22

### 24-Bromo-17S,20S-methylene-fusidic acid (Compound 122)

By following the procedure given in Example 14 and replacing 24-bromo-16-deacetoxy-16β-thioacetyl-fusidic acid acetoxymethylester (111) with 24-Bromo-17S,20S-methylene-fusidic acid acetoxymethyl ester (compound 121), the title compound (122) was obtained. (The FCC-eluant was 50% EtOAc in pet.ether+1% HCOOH). ¹³C NMR, (CDCl₃): 178.3, 170.0, 130.8, 120.9, 79.6, 71.5, 68.3, 49.3, 48.6, 44.5, 40.8, 40.3, 39.8, 37.1, 36.2, 36.1, 35.9, 34.7, 32.5, 30.3, 29.9, 29.0, 25.3, 24.4, 22.7, 21.4, 20.8, 20.4, 20.2, 17.6, 15.9.

### Example 23

### 3-Deoxy-30,24-Dibromo-fusidic acid (Compound 123)

By following the procedure given in Example 14 and replacing 24-bromo-16-deacetoxy-16β-thioacetyl-fusidic acid acetoxymethylester (111) with 3-deoxy 30,24-dibromo-fusidic acid acetoxymethyl ester (compound 144), the title compound (123) was obtained. (The FCC-eluant was 10% EtOAc in pet.ether+1% HCOOH). ¹³C NMR, (CDCl₃): 173.8, 170.4, 153.0, 131.7, 128.2, 120.0, 74.4, 68.2, 62.7, 49.0, 48.8, 45.5, 44.5, 41.3, 39.4, 39.0, 37.7, 37.2, 36.8, 36.1, 35.1, 32.5, 27.9, 25.4, 23.9, 23.8, 22.0, 20.6, 20.4, 18.9, 17.9.

### Example 24

### 3α-Azido-24-Bromo-3-deoxy-fusidic acid (Compound 124)

3-Deoxy-3β,24-Dibromo-fusidic acid (123) (100 mg; 0.15 mmol) was dissolved in DMF (2.5 ml) and lithium azide (30 mg; 0.6 mmol) was added. The mixture was stirred at rt under argon for 11 days. EtOAc and water (5 ml of each) was added, together with AcOH to give a slightly acidic pH. Work-up (EtOAc, water, sat.NaCl), drying with Na₂SO₄ and concentration gave a crude product which was purified by FCC (Eluant: 0% to 50% EtOAc in pet.ether+1% HCOOH) to give the title compound (124). ¹³C NMR, (CDCl₃): 174.1, 170.5, 153.1, 131.6, 128.2, 120.0, 74.5, 68.1, 65.4, 49.1, 48.8, 44.6, 39.4, 39.0, 37.8, 37.4, 36.9, 35.9, 35.5, 32.4, 30.8, 27.9, 26.9, 25.4, 24.2, 23.0, 20.6, 20.5, 20.4, 18.1, 16.7.

### Example 25

### 24-Iodo-fusidic acid (Compound 125)

24-Bromofusidic acid (108) (17.0 g; 28.5 mmol), Cul (27.2 g; 143 mmol), KI (43.4 g; 285 mmol) and HMPA (100 ml) was heated in an oil-bath a for 20 hours at 120° C., under argon, with stirring and with a reflux condenser. Water (400 ml) was added, and the resulting viscous mixture was extracted four times with EtOAc (400 ml in total). The organic phase was filtered through filter aid, which was washed with EtOAc. The combined organic phase was extracted with 20% aqueous Na₂S₂O₅, twice with water, and with sat.NaCl. After drying with MgSO₄ the solvent was evaporated, and the residue was stirred with toluene (300 ml) for three hours. The precipitate was isolated by filtration, washed with toluene and pet.ether and dried to give almost pure title compound 125 as beige-coloured crystals. ¹³C NMR, (CDCl₃): 173.8, 170.6, 152.3, 137.4, 128.0, 99.9, 74.5, 71.5, 68.2, 60.4, 49.3, 48.8, 44.5, 41.7, 39.5, 39.0, 37.0, 36.4, 36.0, 32.1, 31.6, 30.2, 29.9, 24.0, 23.0, 20.9, 20.7, 19.5, 17.9, 15.9, 14.2.

### Example 26

### 24-Iodo-fusidic acid acetoxymethyl ester (Compound 126)

By following the procedure given for example 25 and replacing compound 108 with 24-bromofusidic acid acetoxymethyl ester (107), crude compound 126 was obtained. This was purified by FCC, with 40% EtOAc in pet.ether as eluant, to give the title compound 126 as an amorphous substance. ¹³C NMR, (CDCl₃): 170.3, 169.6, 168.0, 152.5, 137.5, 127.6, 99.8, 79.5, 74.4, 71.4, 68.1, 49.4, 48.8, 44.6, 41.7, 39.5, 39.0, 36.9, 36.5, 35.8, 32.0, 31.6, 30.1, 29.9, 28.6, 23.9, 23.1, 20.9, 20.8, 20.8, 19.4, 17.9, 16.0, 14.2.

### Example 27

### 24-Iodo-fusidic acid pivaloyloxymethyl ester (Compound 127)

24-Iodo-fusidic acid (125) (0.84 g; 1.31 mmol) and triethylamine (0.19 ml; 0.14 g; 1.35 mmol) was dissolved in DMF (5 ml) and stirred for 20 min. at rt. Chloromethyl pivalate (0.30 ml; 0.32 g; 2.1 mmol) was added and the mixture stirred overnight at rt. The reaction mixture was worked up by extraction with 3M aqueous CaCl₂, water and sat.NaCl, dried with MgSO₄ and concentrated. The residue was purified by FCC, with 40% EtOAc in pet.ether as eluant, to give the title compound 127 as an amorphous substance. ¹³C NMR, (CDCl₃): 177.0, 170.2, 167.8, 152.8, 137.5, 127.7, 99.7, 80.1, 74.4, 71.4, 68.2, 60.4, 49.3, 48.8, 44.6 ,41.6, 39.5, 39.0, 38.8, 37.1, 36.3, 36.1, 36.0, 32.3, 31.6, 30.2, 30.0, 28.6, 26.9, 24.1, 22.8, 20.8, 20.8, 19.5, 18.0, 16.0, 14.2.

### Example 28

### Cream

| | | |
|---|---|---|
| 24-Bromo-fusidic acid sodium salt | 1 | g |
| Petrolatum | 7.5 | g |
| Liquid paraffin | 7.5 | g |
| Spermaceti | 2.5 | g |
| Sorbitane monopalmitate | 2.5 | g |
| Polyoxyethylene sorbitane monopalmitate | 2.5 | g |
| Water | 26.5 | g |
| | 50 | g |

Heat petrolatum, paraffin, spermaceti, sorbitane monopalmitate and polyoxyethylene sorbitane monopalmitate to 70° C. and slowly add water under continuous stirring. Continue stirring until the cream has cooled. Triturate 24-Bromo-fusidic acid sodium salt, into the cream base and homogenise using a roller mill. Fill the cream into aluminium collapsible tubes.

### Example 29

### Ointment

| | | |
|---|---|---|
| 24-Bromo-fusidic acid sodium salt | 1 | g |
| Liquid paraffin | 6.9 | g |
| Cetanol | 0.2 | g |
| Lanolin anhydrous | 2.3 | g |
| Petrolatum | 39.6 | g |
| | 50 | g |

Melt paraffin, cetanol, lanolin and petrolatum at 70° C. After cooling to below 40 ° C. triturate 24-Bromo-fusidic acid sodium salt. Fill the ointment into lacquered collapsible aluminium tubes.

### Example 30

### Capsules

| | | |
|---|---|---|
| 24-Chloro-fusidic acid sodium salt | 25 | g |
| Microcrystalline cellulose | 14.5 | g |
| Magnesium stearate | 0.5 | g |
| | 40 | g |

Pass the ingredients through a 60 mesh sieve and mix for 10 min. Fill the mixture into hard gelatine capsules using a capsule fill weight of 400 mg.

### Example 31

### Tablets

| | | |
|---|---|---|
| 24-Bromo-fusidic acid sodium salt | 25 | g |
| Avicel ™ | 12 | g |
| STA-Rx 1500 | 12 | g |
| Magnesium stearate | 1 | g |
| | 50 | g |

16-Deacetoxy-16β-(2′,2′,2′-trifluoroethoxy)- 17S,20S-methanofusidic acid, sodium salt, Avicel™ and STA-Rx are mixed together, sieved through a 0.7 mm sieve and thereafter mixed with magnesium stearate: The mixture is pressed into tablets each of 500 mg.

### Example 32

### Suspension

| | | |
|---|---|---|
| 24-Bromo-16-deacetoxy-16β-isopropylsulfinyl-fusidic | 1 | g |
| acid sodium salt | | |
| Citric acid | 0.09 | g |
| Sodium monohydrogenphosphate | 0.14 | g |
| Sucrose | 5 | g |
| Tween ™ 80 | 0.01 | g |
| Potassium sorbate | 0.04 | g |
| Carboxymethylcellulose-Na | 0.1 | g |

| | |
|---|---|
| Water suspension. | qs. to 100 ml |

The crystals are micronized and suspended in a solution of citric acid, sodium monohydrogen phosphate, sucrose, potassium sorbate and Tween™ 80 in 10 ml water, if necessary with slight warming. Carboxymethylcellulose-Na is dissolved in 4 ml boiling water. After cooling, it is added to the other Ingredients. The suspension is homogenised in a blender and finally water is added to a total volume of 100 ml.

### Example 33

### Ointment

| | | |
|---|---|---|
| A: 24-Bromo-16-deacetoxy-16β-(2′,2′,2′- | 1 | g |
| trifluoroethoxy)-fusidic acid sodium salt | | |
| B: One of the compounds: hydrocortisone, | 0.5 | g |
| triamcinolone or fluocinolone | | |
| Liquid paraffin | 6.9 | g |
| Cetanol | 0.2 | g |
| Lanolin anhydrous | 2.3 | g |
| Petrolatum | 39.1 | g |
| | 50 | g |

Melt paraffin, cetanol, lanolin and petrolatum at 70° C. After cooling to below 40° C., triturate A and B. Fill the ointment into lacquered collapsible aluminium tubes.

### Example 34

### Ointment

| | | |
|---|---|---|
| A: 24-Bromo-17S,20S-dihydrofusidic acid | 1.5 | g |
| B: Tetracycline | 1.5 | g |
| Liquid paraffin | 13.8 | g |
| Cetanol | 0.4 | g |
| Lanolin anhydrous | 4.6 | g |
| Petrolatum | 78.2 | g |
| | 100 | g |

Melt paraffin, cetanol, lanolin and petrolatum at 70° C. After cooling to below 40° C., triturate A and B. Fill the ointment into lacquered collapsible aluminium tubes.

### Example 35

### Eye Gel

| | | |
|---|---|---|
| 24-Bromo-16-deacetoxy-16β-(2′,2′,2′- | 10 | g |
| trifluoroethoxy)-fusidic acid | | |
| Benzalkonium chloride | 0.1 | g |
| Carbomer | 5 | g |
| Mannitol | 50 | g |
| Sodium edetate | 0.5 | g |

| | |
|---|---|
| Sodium hydroxide | q.s. |
| Sterile water | up to 100 g |

Dissolve disodium edetate and mannitol in water for injection in a stainless steel vessel equipped with a stirring tool and a built-in homogenizer. Add Carbomer 934P, evacuate the vessel and autoclave the dispersion under slow stirring and homogenizing at high speed. Cool down to 70° C., stop agitator and homogenizer. Add 24-Bromo-16-deacetoxy-16β-(2′,2′,2′-trifluoroethoxy)-fusidic acid, sodium salt micronized, sterile—evacuate the vessel and let the 24-Bromo-16-deacetoxy-16β-(2′,2′,2′-trifluoroethoxy)-fusidic acid sink during slow agitation. Homogenize at high speed for 10 minutes at 70° C. Cool down to below 30° C. during stirring and homogenizing at low speed. Add a sterile solution of benzalkonium chloride in water for injection under slow stirring. Neutralise the carbomer 934 P by adding a sterile solution of sodium hydroxide 1.050 kg in water for injection. Stir and homogenize at low speed for 5 minutes. Adjust—if necessary—the pH to 5.4-5.8. Transfer the eye gel to storage tanks using nitrogen pressure and the low speed homogenizing transfer system. Store at room temperature until filling. The eye gel is filled aseptically in sterile tubes using a fill weight of 3.5 g.

### Example 36

### 24-Phenyl-fusidic acid pivaloyloxymethylester (Compound 136)

Phenylboronic acid (50 mg, 0.4 mmol) and EtOH (0.25 ml) was added to a solution of 24-iodo-fusidic acid pivaloyloxymethylester (127) (150 mg, 0.2 mmol) in toluene (1.5 ml) and argon was bubbled through the mixture for 2 min. K₂CO₃ (2M aq. solution, 0.3 ml) and Pd(PPh₃)₄ (11.5 mg, 0.01 mmol) were added, and the mixture was shaken at 90° C. for 20 hours under argon. The reaction mixture was worked up with EtOAc, water and sat.NaCl, dried and concentrated. The resulting crude product was purified by FCC (20% EtOAc in pet.ether as eluant) to give the pure title compound 136. ¹³C NMR, (CDCl₃): 177.0, 170.2, 167.7, 152.3, 144.1, 133.9, 129.6, 128.9, 128.4, 127.9, 125.9, 80.0, 74.3, 71.4, 67.9, 48.9, 48.7, 44.4, 39.4, 39.0, 38.8, 36.9, 36.3, 36.1, 35.4, 35.0, 32.2, 30.0, 27.4, 26.9, 23.9, 22.7, 22.0, 20.8, 20.8, 20.0, 17.9, 15.9.

### Example 37

### 24-Phenyl-fusidic acid (Compound 137)

By following the procedure of example 3 and replacing 24-chloro-fusidic acid pivaloyloxymethyl ester (104) with 24-phenyl-fusidic acid pivaloyloxymethylester (136), and inserting an aqueous work up procedure (EtOAc, water+aq. HCl to pH ca. 2 and sat.NaCl) before the FCC, the pure title compound 137 was obtained. ¹³C NMR, (CDCl₃):173.9, 170.6, 151.7, 144.1, 134.0, 129.6, 129.5, 128.4, 127.9, 125.9, 74.4, 71.5, 67.9, 48.9, 48.6, 44.3, 39.4, 39.0, 36.8, 36.3, 36.1, 35.4, 35.0, 32.2, 30.0, 30.0, 27.4, 23.8, 22.8, 22.0, 20.8, 20.7, 20.0, 17.9, 15.9.

### Example 38

### 24-(4-Bromophenyl)-fusidic acid pivaloyloxymethylester (Compound 138)

By following the procedure of example 36 and replacing phenylboronic acid with [2-(4-bromophenyl)-5,5-dimethyl-1,3,2-dioxaborinane] the title compound 138 was obtained.

¹³C NMR, (CDCl₃):177.0, 170.2, 167.7, 152.5, 142.9, 132.9, 131.3, 131.1, 129.0, 128.6, 119.9, 80.0, 74.3, 71.4, 68.0, 49.0, 48.6, 44.5, 39.4, 39.0, 38.8, 37.0, 36.2, 35.1, 35.0, 32.4, 30.1, 30.0, 27.4, 26.9, 24.1, 22.7, 22.0, 20.8, 20.7, 20.0, 18.0, 15.9

### Example 39

### 24-(4-Bromophenyl)-fusidic acid (Compound 139)

By following the procedure of example 3 and replacing 24-chloro-fusidic acid pivaloyloxymethyl ester (104) with 24-(4-bromophenyl)-fusidic acid pivaloyloxymethylester (138), and inserting an aqueous work up procedure (EtOAc, water+aq.HCI to pH ca. 2 and sat.NaCl) before the FCC, the pure title compound 139 was obtained. ¹³C NMR, (CDCl₃): 174.0, 170.6, 152.1, 142.9, 133.0, 131.3, 131.1, 129.1, 128.4, 119.8, 74.3, 71.5, 68.0, 49.0, 48.6, 44.4, 39.4, 39.0, 37.0, 36.2, 35.1, 35.0, 32.3, 30.1, 30.0; 27.4, 24.0, 22.7, 22.1, 20.8, 20.6, 20.0, 17.9, 15.9.

### Example 40

### 24-(4-Chlorophenyl)-fusidic acid pivaloyloxymethylester (Compound 140)

By following the procedure of example 36 and replacing phenylboronic acid with [2-(4-chlorophenyl)-5,5-dimethyl-1,3,2-dioxaborinane] the title compound 140 was obtained.

¹³C NMR, (CDCl₃): 177.0, 170.2, 167.8, 152.3, 142.4, 132.9, 131.8, 130.9, 129.1, 128.7, 128.2, 80.0, 74.2, 71.4, 68.0, 49.0, 48.6, 44.5, 39.4, 39.0, 37.0, 36.2, 36.1, 35.1, 35.0, 32.3, 30.1, 30.0, 27.4, 26.9, 24.0, 22.7, 22.0, 21.3, 20.8, 20.8, 20.0, 17.9, 15.9

### Example 41

### 24-(4-Chlorophenyl)-fusidic acid (Compound 141)

By following the procedure of example 3 and replacing 24-chloro-fusidic acid pivaloyloxymethyl ester (104) with 24-(4-chlorophenyl)-fusidic acid pivaloyloxymethylester (140), and inserting an aqueous work up procedure (EtOAc, water+aq.HCl to pH ca. 2 and sat.NaCl) before the FCC, the pure title compound 141 was obtained. ¹³C NMR, (CDCl₃): 173.9, 170.6, 151.9, 142.4, 133.0, 131.8, 130.9, 129.2, 129.1, 128.1, 74.3, 71.6, 71.5, 68.0, 49.0, 48.6, 44.4, 39.4, 39.0, 37.0, 36.2, 36.1, 35.0, 32.3, 30.0, 27.4, 24.0, 22.7, 22.1, 21.3, 20.8, 20.6, 20.0, 17.9, 15.9.

### Example 42

### 24-(3,5-Difluorophenyl)-fusidic acid pivaloyloxymethylester (Compound 142)

By following the procedure of example 36 and replacing phenylboronic acid with [2-(3,5-difluorophenyl)-5,5-dimethyl-1,3,2-dioxaborinane] the title compound 142 was obtained.

¹³C NMR, (CDCl₃): 177.0, 170.2, 167.8, 162.9, 162.7, 152.2, 147.2, 132.3, 130.1, 128.5, 112.2, 112.0, 101.4, 80.1, 74.2, 71.4, 68.1, 49.1, 48.6, 44.5, 39.4, 39.0, 38.8, 37.0, 36.2, 35.3, 34.4, 32.4, 30.2, 30.0, 27.6, 26.9, 24.2, 22.6, 22.1, 20.8, 20.7, 20.2, 18.0, 15.9.

### Example 43

### 24-(3,5-Difluorophenyl)-fusidic acid (Compound 143)

By following the procedure of example 3 and replacing 24-chloro-fusidic acid pivaloyloxymethyl ester (104) with 24-(3,5-difluorophenyl)-fusidic acid pivaloyloxymethylester (142), and inserting an aqueous work up procedure (EtOAc, water+aq.HCl to pH ca. 2 and sat.NaCl) before the FCC, the pure title compound 143 was obtained. ¹³C NMR, (CDCl₃): 174.1, 170.6, 162.8, 162.7, 152.0, 147.2, 132.3, 130.2, 129.0, 112.2, 101.4, 74.3, 71.5, 68.1, 49.1, 48.6, 44.4, 39.4, 39.0, 37.0, 36.2, 36.2, 35.3, 34.5, 32.4, 30.2, 29.9, 27.5, 24.1, 22.7, 22.1, 20.7, 20.6, 20.1, 17.9, 15.9.

### Example 44

### 3-Deoxy-3β,24-Dibromo-fusidic acid acetoxymethyl ester (Compound 144)

24-Bromo-fusidic acid acetoxymethyl ester (107) (0.45 g; 0.67 mmol) was dissolved in dry benzene (10 ml) and stirred at rt under argon. Triphenylphosphine (0.7 g; 2.7 mmol) and tetrabromomethane (1.1 g; 3.3 mmol) were added, and the mixture was stirred for one hour at rt. Ether (50 ml) was added, and the precipitated material was removed by filtration. The filtrate was concentrated, and the residue was purified by FCC (eluant: 0% to 50% EtOAc in pet.ether) to give the title compound (144). ¹³C NMR, (CDCl₃): 170.3, 169.6, 167.8, 152.3, 131.8, 128.1, 119.9, 79.5,74.3, 68.1, 62.7, 49.0, 48.8, 45.5, 44.4, 41.3, 39.4, 39.0, 37.7, 37.2, 36.8, 36.1, 35.1, 32.5, 27.8, 25.3, 23.9, 23.9, 22.0, 20.8, 20.7, 20.4, 18.9, 17.9.

### Example 45

### 24-Bromo-fusidic acid (Compound 108)

24,25-dibromo-fusidic acid (17) (the crude product from 0.1 mol fusidic acid) was dissolved in EtOH (900 ml) and water (25 ml) and K₂CO₃ (30 g, 0.22 mol) were added. This mixture was refluxed with continuous stirring for 30 minutes, cooled to rt, and poured into water (4 liters).The alkaline solution of the potassium salt of (108) was acidified by addition of aq. H₃PO₄ (350 ml, 1M), under continuous stirring, to give a pH of 4.0, whereby a precipitate was formed. The product was collected by filtration, washed with water and dried to give crude (108). The crude compound 108 may then be either purified and recrystallized, e.g. as described in example 8 to give the pure compound 108, or converted into an easily hydrolysable ester, e.g. using the procedure described In preparations 1 and 2, or converted into a suitable salt, such as a sodium salt, e.g. as described in example 9. An advantage of preparing the sodium salt of compound 108 is that a particularly pure crystalline sodium salt is formed directly, without the need of chromatographic purification. By liberating the free acid from this sodium salt (e.g. in the same way as described above for the potassium salt) a product is obtained which can be crystallized directly (e.g. from ethyl acetate and toluene) to give the pure crystalline compound 108.

### Example 46

### 24-Bromo-16-deacetoxy-16β-ethylthio-fusidic acid (Compound 146)

By following the procedure given In Example 12 and replacing 2-propanethiol with ethanethiol the title compound (146) was obtained. ¹³C NMR, (CDCl₃): 176.3, 154.8, 131.3, 128.3, 120.4, 71.5, 68.4, 49.4, 48.9, 46.1, 45.6, 41.0, 39.7, 37.9, 37.1, 36.4, 36.0, 35.8, 32.7, 30.4, 30.0, 29.6, 28.6, 25.3, 24.4, 22.5, 20.7, 20.4, 18.8, 16.0, 14.7.

### Example 47

### 24-Bromo-16-deacetoxy-16β-ethylsulfinyl-fusidic acid (Compound 147)

By following the procedure given in Example 13 and replacing 24-bromo-16-deacetoxy-16β-isopropylthio-fusidic acid (112) with 24-bromo-16-deacetoxy-16β-ethylthio-fusidic acid (Compound 146) the title compound (147) was obtained. ¹H NMR (CDCl₃): 4.40 (d, 1H), 4.37 (m, 1H), 3.76 (m, 1H), 3.18 (d, 1H), 2.90-2.40 (m, 5H), 2.25-1.0 (m, 19H), 1.84 (bs, 3H), 1.77 (bs, 3H), 1.45 (s, 3H), 1.25 (t, 3H), 0.97 (s, 3H), 0.93 (d, 3H), 0.79 (s, 3H).

### Example 48

### 24-Bromo-16-deacetoxy-16β-allylthio-fusidic acid (Compound 148)

By following the procedure given in Example 12 and replacing 2-propanethiol with allyl mercaptan the title compound (148) was obtained. ¹³C NMR, (CDCl₃): 175.5, 154.7, 134.4, 131.3, 128.4, 120.4, 117.0, 71.5, 68.4, 49.4, 48.9, 45.6, 39.7, 37.9, 37.1, 36.4, 36.0, 34.7, 32.7, 30.9, 30.3, 30.0, 29.7, 29.0, 28.6, 25.4, 24.4, 22.5, 20.7, 20.4, 18.9, 16.0.

### Example 49

### 24-Bromo-16-deacetoxy-16β-(1-pentylthio)-fusidic acid (Compound 149)

By following the procedure given in Example 12 and replacing 2-propanethiol with 1-pentanethiol the title compound (146) was obtained. ¹³C NMR, (CDCl₃): 176.0, 155.0, 131.3, 128.3, 120.4, 71.5, 68.4, 49.4, 48.9, 46.6, 45.6, 41.0, 39.7, 38.0, 37.1, 36.3, 36.0, 35.9, 35.9, 32.7, 31.4, 30.3, 30.0, 29.4, 28.7, 25.3, 24.3, 22.5, 22.3, 20.7, 20.4, 18.8, 16.0, 14.0.

### Example 50

### 24-Bromo-16-deacetoxy-16β- (1-pentylsulfinyl)-fusidic acid (Compound 150)

By following the procedure given in Example 13 and replacing 24-bromo-16-deacetoxy-16β-isopropylthio-fusidic acid (112) with 24-bromo-16-deacetoxy-16β-(1-pentylthio)-fusidic acid (Compound 149) the title compound (150) was obtained. ¹³C NMR, (CDCl₃): 173.6, 159.2, 131.3, 125.9, 120.2, 71.5, 68.3, 49.5, 48.2, 47.6, 39.8, 38.1, 37.1, 36.2, 35.5, 32.5, 31.0, 30.3, 29.9, 27.9, 25.8, 25.3, 24.5, 22.8, 22.7, 22.4, 20.7, 20.4, 17.8, 16.0, 14.0.

### Example 51

### 24-Bromo-16-deacetoxy-16β-(2-methyl-1-butylthio)-fusidic acid (Compound 151)

By following the procedure given in Example 12 and replacing 2-propanethiol with 2-methyl-1-butanethiol the title compound (151) was obtained.

¹³C NMR, (CDCl₃): 175.8, 155.0, 131.3, 128.3, 120.4, 71.6, 68.4, 49.4, 48.9, 47.2, 46.8, 45.7, 45.6, 43.4, 42.9, 40.8, 40.4, 39.7, 38.0, 37.1, 36.4, 36.0, 35.9, 34.9, 34.6, 32.8, 30.4, 30.0, 29.3, 28.7, 25.3, 24.4, 22.5, 20.7, 20.4, 19.3, 19.0, 18.9, 16.0, 11.3.

### Example 52

### 24-Bromo-16-deacetoxy-16β-(2-methyl-1-butylsulfinyl)-fusidic acid (Compound 152)

By following the procedure given in Example 13 and replacing 24-bromo-16-deacetoxy-16β-isopropylthio-fusidic acid (112) with 24-bromo-16-deacetoxy-16β-(2-methyl-1-butylthio)-fusidic acid (Compound 151) the title compound (152) was obtained. ¹³C NMR, (CDCl₃): 173.9, 131.3, 126.0, 120.2, 71.5, 68.3, 49.6, 48.3, 47.8, 39.8, 38.2, 37.1, 36.2, 36.1, 35.5, 32.4, 30.3, 29.9, 28.0, 27.9, 25.3, 24.5, 22.8, 20.8, 20.4, 19.5, 17.9, 16.0, 11.2.

### Example 53

### 24-Bromo-16-deacetoxy-16β-(3-methyl-1-butylthio)-fusidic acid (Compound 153)

By following the procedure given in Example 12 and replacing 2-propanethiol with 3-methyl-1-butanethiol the title compound (153) was obtained. ¹³C NMR, (CDCl₃): 176.0, 155.0, 131.2, 128.3, 120.5, 71.6, 68.4, 49.4, 48.9, 46.6, 45.7, 40.9, 39.7, 38.5, 38.0, 37.1, 36.3, 36.1, 35.9, 33.9, 32.6, 30.3, 30.0, 28.7, 27.7, 25.3, 24.3, 22.6, 22.4, 22.3, 20.8, 20.4, 18.8, 16.0.

### Example 54

### 24-Bromo-16-deacetoxy-16β-(3-methyl-1-butylsulfinyl)-fusidic acid (Compound 154)

By following the procedure given in Example 13 and replacing 24-bromo-16-deacetoxy-16β-isopropylthio-fusidic acid (112) with 24-bromo-16-deacetoxy-16β-(3-methyl-1-butylthio)-fusidic acid (Compound 153) the title compound (154) was obtained.

¹³C NMR, (CDCl₃): 173.6, 131.3, 120.2, 71.5, 68.3, 49.5, 48.2, 39.8, 38.1, 37.1, 36.2, 35.6, 32.5, 31.5, 30.3, 29.9, 27.8, 25.3, 24.5, 22.8, 22.6, 2.3, 20.8, 20.4, 17.8, 16.0.

### Example 55

### 24-Bromo-16-deacetoxy-16β-cyclopentylthio-fusidic acid (Compound 155)

A solution of bromine (12.0 mg, 0.150 mol) in ethyl acetate (0.6 ml) was added to a solution of 16-deacetoxy-16β-cyclopentylthio-fusidic acid (von Daehne, W. et al., Adv. Appl. Microbiol., 1979, vol. 25, p. 95-146) (76.0 mg, 0.136 mmol) in ethyl acetate (4 ml), during 2 minutes, with stirring and cooling in an ice bath. 1M aq. KH₂PO₄ (0.2 ml) and 1M aq. Na₂S₂O₃ (0.1 ml) was added, with stirring for a five minutes. The EtOAc-phase was separated and extracted with 0.5M aq. KH₂PO₄ (0.5 ml) and with water (0.5 ml) and concentrated to give the intermediate 24,25-dibromo fusidic acid analog as an oil which was used without further purification for dehydrobromination. The intermediate 24,25-dibromo fusidic acid analog was dissolved in ethanol (3 ml); water (0.06 ml) and K₂CO₃ (40 mg, 0.3 mmol) was added and the mixture was refluxed, with stirring, for ½ h, cooled to rt, and poured into water (15 ml). The alkaline solution of (155) was acidified by addition of 1M aq. H₃PO₄, with stirring, to give a pH of 4.0, and worked up (EtOAc, sat.NaCl) to give a crude product. The crude product was purified by FCC (10% to 50% EtOAc in petr.ether+1% AcOH, as eluant) to give the pure title compound (155).

¹³C NMR, (CDCl₃): 175.3, 155.0, 131.3, 128.2, 120.4, 71.5, 68.4, 49.4, 48.8, 47.7, 46.2, 45.6, 41.1, 39.7, 38.0, 37.2, 36.5, 35.9, 34.6, 32.9, 32.5, 30.4, 30.1, 28.8, 25.3, 24.8, 24.6, 24.5, 22.3, 20.7, 20.4, 19.0, 16.0.

### Example 56

### 24-Bromo-16-deacetoxy-16β-(2,2,2-trifluoroethylthio)-fusidic acid (Compound 156)

By following the procedure given in Example 12 and replacing 2-propanethiol with 2,2,2-trifluoroethanethiol the title compound (156) was obtained. ¹³C NMR, (CDCl₃): 175.6, 155.6, 131.5, 128.9, 120.2, 71.5, 68.3, 49.1, 48.9, 45.9, 41.2, 39.6, 38.9, 37.8, 37.1, 36.3, 36.0, 35.8, 32.7, 30.4, 30.0, 28.5, 25.3, 24.4, 22.5, 21.1, 20.6, 20.4, 18.9, 16.0.

### Example 57

### 24-Bromo-16-deacetoxy-16β-(2-hydroxyethylthio)-fusidic acid (Compound 157)

By following the procedure given in Example 55 and replacing 16-deacetoxy-16β-cyclopentylthio-fusidic acid with 16-deacetoxy-16β-(2-hydroxyethyl thio)-fusidic acid (von Daehne, W. et al., Adv. Appl. Microbiol., 1979, vol. 25, p. 95-146), the title compound (157) was obtained. ¹³C NMR, (CDCl₃): 174.4, 153.3, 131.3, 129.0, 120.5, 71.6, 68.3, 59.0, 49.4, 48.9, 45.7, 43.9, 39.9, 39.8, 37.9, 37.1, 36.7, 36.3, 36.1, 35.8, 32.6, 30.3, 30.0, 28.5, 25.4, 24.3, 22.6, 20.7, 20.4, 18.9, 16.0.

### Example 58

### 24-Bromo-16-deacetoxy-16β-benzylthio-fusidic acid (Compound 158)

By following the procedure given in Example 12 and replacing 2-propanethiol with benzyl mercaptan the title compound (158) was obtained. ¹³C NMR, (CDCl₃): 175.9, 155.0, 138.4, 131.3, 129.0, 128.4, 128.3, 126.8, 120.4, 71.6, 68.4, 49.3, 48.9, 46.0, 45.7, 40.1, 39.9, 39.7, 38.0, 37.1, 36.3, 36.0, 35.8, 32.5, 30.3, 30.0, 28.7, 25.3, 24.2, 22.5, 20.7, 20.4, 18.9, 16.0.

### Example 59

### 24-Bromo-16-deacetoxy-16β-benzylsulfinyl-fusidic acid (Compound 159)

By following the procedure given in Example 13 and replacing 24-bromo-16-deacetoxy-16β-isopropylthio-fusidic acid (112) with 24-bromo-16-deacetoxy-16β-benzylthio-fusidic acid (Compound 158) the title compound (159) was obtained. ¹H NMR (CDCl₃): 7.30 (m, 5H), 4.54 (d, 1H), 4.35 (bs, 1H), 4.04 (d, 1H), 3.76 (d, 1H), 3.76 (m, 1H), 3.15 (bd, 1H), 2.80-2.4 (m, 4H), 2.20-1.0 (m, 18H), 1.84 (bs, 3H), 1.76 (bs, 3H), 1.38 (s, 3H), 0.96 (s, 3H), 0.92 (d, 3H), 0.75 (s, 3H).

### Example 60

### 24-Bromo-16-deacetoxy-16β-(2-furylmethylthio)-fusidic acid (Compound 160)

By following the procedure given in Example 12 and replacing 2-propanethiol with furfuryl mercaptan the title compound (160) was obtained. ¹³C NMR, (CDCl₃): 175.6, 154.8, 151.9, 141.9, 131.3, 128.4, 120.4, 110.5, 107.5, 71.6, 68.4, 60.4, 49.3, 48.9, 46.6, 45.6, 40.4, 39.7, 37.9, 37.0, 36.2, 36.1, 35.8, 32.4, 32.2, 30.3, 30.0, 25.3, 24.2, 22.6, 21.0, 20.7, 20.4, 18.7, 16.0.

### Example 61

### 24-Bromo-16-deacetoxy-16β-phenylthio-fusidic acid (Compound 161)

By following the procedure given in Example 12 and replacing 2-propanethiol with thiophenol the title compound (161)was obtained. ¹³C NMR, (CDCl₃): 176.6, 175.2, 153.1, 138.3, 131.5, 129.1, 129.0, 128.8, 126.0, 120.2, 71.6, 68.4, 49.5, 48.8, 48.5, 45.8, 39.8, 37.9, 37.1, 36.4, 35.9, 35.8, 32.8, 30.3, 30.0, 28.6, 25.3, 24.4, 22.4, 20.7, 20.4, 19.3, 16.0.

### Example 62

### 24-Bromo-16-deacetoxy-16β-benzoylthio-fusidic acid (Compound 162)

By following the procedure given in Example 55 and replacing 16-deacetoxy-16β-cyclopentylthio-fusidic acid with 16-deacetoxy-16β-benzoylthio-fusidic acid (von Daehne, W. et al., Adv. Appl. Microbiol., 1979, vol. 25, p. 95-146), the title compound (162) was obtained. ¹³C NMR, (CDCl₃): 191.4, 173.6, 154.1, 137.0, 133.0, 131.5, 128.5, 128.3, 127.3, 120.2, 71.5, 68.3, 49.3, 49.0, 46.1, 43.9, 41.5, 39.7, 37.8, 37.2, 36.4, 35.9, 32.7, 30.4, 30.0, 28.4, 25.3, 24.5, 22.4, 20.6, 20.4, 19.0, 16.0.

### Example 63

### 24-Bromo-16-deacetoxy-16β-isopropoxy-fusidic acid (Compound 163)

By following the procedure given in Example 55 and replacing 16-deacetoxy-16β-cyclopentylthio-fusidic acid with 16-deacetoxy-16β-isopropoxy-fusidic acid (von Daehne, W. et al., Adv. Appl. Microbiol., 1979, vol. 25, p. 95-146), the title compound (163) was obtained. ¹³C NMR, (CDCl₃): 171.6, 151.7, 131.9, 131.5, 120.2, 71.5, 70.1, 68.4, 49.3, 49.0, 44.2, 39.7, 37.6, 37.2, 36.4, 35.9, 35.3, 32.8, 30.3, 30.1, 28.9, 25.3, 24.3, 23.2, 22.4, 20.7, 20.5, 20.0, 18.4, 16.0.

### Example 64

### 24-Bromo-16-deacetoxy-16β-(2-fluoroethoxy)-fusidic acid (Compound 164)

By following the procedure given in Example 55 and replacing 16-deacetoxy-16β-cyclopentylthio-fusidic acid with 16-deacetoxy-16β-(2-fluoroethoxy)-fusidic acid (von Daehne, W. et al., Adv. Appl. Microbiol., 1979, vol. 25, p. 95-146), the title compound (164) was obtained. ¹³C NMR, (CDCl₃): 174.0, 151.8, 131.4, 129.6, 120.3, 82.5, 80.1, 71.5, 69.1, 68.4, 49.2, 49.1, 43.8, 39.6, 37.7, 37.1, 36.3, 36.1, 35.7, 32.6, 30.3, 30.0, 28.3, 25.3, 24.3, 22.6, 20.8, 20.4, 18.0, 16.0.

### Example 65

### 24-Bromo-16-deacetoxy-16β-(2-methoxyethoxy)-fusidic acid (Compound 165)

By following the procedure given in Example 55 and replacing 16-deacetoxy-16β-cyclopentylthio-fusidic acid with 16-deacetoxy-16β-(2-methoxyethoxy)-fusidic acid (von Daehne, W. et al., Adv. Appl. Microbiol., 1979, vol. 25, p. 95-146), the title compound (165) was obtained. ¹³C NMR, (CDCl₃): 172.1, 151.3, 131.4, 131.0, 120.3, 80.7, 71.6, 71.5, 68.8, 68.4, 59.0, 49.4, 49.1, 43.9, 39.7, 37.6, 37.1, 36.3, 36.1, 35.8, 35.3, 32.6, 30.3, 30.1, 28.6, 25.3, 24.2, 22.6, 20.8, 20.4, 18.2, 16.0.

### Example 66

### 24-(trans-1-Hexen-1-yl)-fusidic acid (Compound 166)

A suspension of 24-(trans-1-hexen-1-yl)-fusidic acid pivaloyloxymethyl ester (306) (50 mg, 0.070 mmol) in MeOH (1 ml) was cooled in an ice bath and K₂CO₃ (20 mg, 0.14 mmol) was added. The mixture was stirred for 3 hours at rt and then worked up (EtOAc, water+aq. HCl to pH ca. 2 and sat.NaCl) to yield a crude product. By FCC of the crude product (pet.ether:EtOAc:HCOOH, 90:10:0 to 0:99:1 as eluant), the pure title compound (166) was obtained. ¹³C NMR, (CDCl₃): 174.1, 170.6, 149.9, 129.9, 129.8, 129.7, 128.5, 127.9, 74.5, 71.5, 68.2, 49.2, 48.7, 44.2, 39.5, 39.0, 37.0, 36.2, 36.2, 35.7, 33.2, 32.4, 32.1, 30.3, 29.9, 28.0, 27.8, 24.2, 22.8, 22.3, 21.5, 20.8, 20.6, 20.4, 17.8, 15.9, 14.0.

### Example 67

### 24-(trans-1-Buten-3,3-dimethyl-1-yl)-fusidic acid (Compound 167)

By following the procedure given in Example 66 and replacing 24-(trans-1-hexen-1-yl)-fusidic acid pivaloyloxymethyl ester (306) with 24-(Trans-1-buten-3,3-dimethyl-1-yl)-fusidic acid pivaloyloxymethyl ester (307) the title compound (167) was obtained. ¹³C NMR, (CDCl₃): 171.1, 139.2, 130.0, 129.9, 122.5, 74.5, 71.5, 68.2, 49.2, 48.7, 44.1, 39.4, 39.0, 37.0, 36.3, 36.2, 35.7, 33.4, 32.4, 30.2, 30.0, 27.9, 24.2, 22.8, 21.6, 20.8, 20.5, 17.8, 15.9.

### Example 68

### 24-(trans-1-Nonen-1-yl)-fusidic acid (Compound 168)

By following the procedure given in Example 66 and replacing 24-(trans-1-hexen-1-yl)-fusidic acid pivaloyloxymethyl ester (306) with 24-(Trans-1-nonen-1-yl)-fusidic acid pivaloyloxymethyl ester (308) the title compound (168) was obtained.

¹³C NMR, (CDCl₃): 173.8, 170.6, 149.9, 129.9, 129.8, 129.7, 128.5, 127.9, 74.5, 71.4, 68.3, 49.2, 48.7, 44.2, 39.5, 39.0, 37.1, 36.2, 35.7, 33.6, 32.4, 31.9, 30.3, 30.0, 29.9, 29.3, 28.0, 27.8, 24.2, 22.7, 21.5, 20.8, 20.6, 20.4, 17.8, 15.9, 14.1.

### Example 69

### 24-(trans-5-Chloro-1-penten-1-yl)-fusidic acid (Compound 169)

By following the procedure given in Example 66 and replacing 24-(trans-1-hexen-1-yl)-fusidic acid pivaloyloxymethyl ester (306) with 24-(trans-5-chloro-1-penten-1-yl)-fusidic acid pivaloyloxymethyl ester (309) the title compound (169) was obtained. ¹³C NMR, (CDCl₃): 174.1, 170.6, 150.2, 130.6, 129.6, 129.3, 125.9, 74.5, 71.5, 68.2, 49.2, 48.7, 44.6, 44.3, 39.5, 39.0, 37.1, 36.2, 35.7, 32.6, 32.4, 30.5, 30.3, 29.9, 27.9, 27.8, 24.2, 22.8, 21.5, 20.8, 20.6, 20.4, 17.8, 15.9.

### Example 70

### 24-(trans-2-Phenyl-1-vinyl)-fusidic acid (Compound 170)

By following the procedure given in Example 66 and replacing 24-(trans-1-hexen-1-yl)-fusidic acid pivaloyloxymethyl ester (306) with 24-(trans-2-phenyl-1-vinyl)-fusidic acid pivaloyloxymethyl ester (310) the title compound (170) was obtained. ¹³C NMR, (CDCl₃): 174.4, 170.6, 150.6, 138.5, 133.6, 130.4, 129.5, 128.6, 127.2, 126.9, 126.3, 126.1, 74.5, 71.5, 68.1, 49.2, 48.7, 44.3, 39.4, 39.0, 37.0, 36.3, 36.1, 35.9, 32.3, 30.1, 29.9, 27.9, 24.0, 22.8, 21.9, 20.8, 20.7, 20.6, 17.8, 15.9.

### Example 71

### 24-(2-Phenyl-1-ethyl)-fusidic acid (Compound 171)

By following the procedure given in Example 66 and replacing 24-(trans-1-hexen-1-yl)-fusidic acid pivaloyloxymethyl ester (306) with 24-(2-phenyl-1-ethyl)-fusidic acid pivaloyloxymethyl ester (311) the title compound (171) was obtained. ¹³C NMR, (CD₃OD): 174.5, 172.6, 147.8, 144.2, 144.1, 132.8, 129.4, 129.4, 129.3, 126.6, 75.7, 72.4, 68.5, 50.7, 44.8, 44.3, 40.7, 40.0, 38.2, 37.8, 37.4, 37.3, 36.7, 35.0, 34.8, 33.7, 33.3, 32.8, 31.6, 31.6, 31.0, 30.9, 30.3, 30.2, 28.1, 23.9, 23.8, 22.4, 20.7, 19.7, 19.7, 19.5, 19.4, 18.0, 16.5.

### Example 72

### 24-(4-n-Propylphenyl)-fusidic acid (Compound 172)

By following the procedure given in Example 66 and replacing 24-(trans-1-hexen-1-yl)-fusidic acid pivaloyloxymethyl ester (306) with 24-(4-n-propyl-phenyl)-fusidic acid pivaloyloxymethyl ester (312) the title compound (171) was obtained. ¹³C NMR, (CDCl₃): 173.3, 170.6, 152.0, 141.4, 140.5, 133.9, 129.4, 128.2, 127.8, 74.5, 71.5, 67.9, 48.9, 48.7, 44.3, 39.4, 39.0, 37.7, 36.9, 36.3, 36.1, 35.5, 35.0, 32.2, 30.1, 30.0, 27.5, 24.7, 23.8, 22.7, 22.1, 20.8, 20.7, 19.9, 18.0, 15.9, 14.0.

### Example 73

### 24-(4-Vinylphenyl)-fusidic acid (Compound 173)

By following the procedure given in Example 66 and replacing 24-(trans-1-hexen-1-yl)-fusidic acid pivaloyloxymethyl ester (306) with 24-(4-vinyl-phenyl)-fusidic acid pivaloyloxymethyl ester (313) the title compound (173) was obtained. ¹³C NMR, (CDCl₃): 173.5, 170.5, 152.5, 143.8, 136.4, 135.4, 133.7, 129.9, 129.0, 128.5, 125.8, 113.5, 74.4, 71.5, 67.8, 48.9, 48.6, 44.4, 39.4, 39.0, 36.9, 36.3, 36.0, 35.3, 35.1, 32.1, 30.0, 27.4, 23.8, 22.7, 22.1, 20.8, 20.7, 19.9, 17.9, 15.9.

### Example 74

### 24-(4-tert-Butylphenyl)-fusidic acid (Compound 174)

By following the procedure given in Example 66 and replacing 24-(trans-1-hexen-1-yl)-fusidic acid pivaloyloxymethyl ester (306) with 24-(4-tert-butylphenyl)-fusidic acid pivaloyloxymethyl ester (314) the title compound (174) was obtained. ¹³C NMR, (CDCl₃): 173.6, 170.6, 151.8, 149.0, 141.0, 133.8, 129.6, 129.1, 128.3, 124.6, 74.5, 71.5, 67.9, 48.9, 48.8, 44.2, 39.4, 39.0, 36.9, 36.2, 36.1, 35.5, 34.9, 34.5, 32.3, 31.5, 30.3, 29.9, 27.6, 23.9, 22.7, 22.1, 20.8, 20.7, 19.9, 18.0, 15.9.

### Example 75

### 24-(4-Cyanophenyl)-fusidic acid (Compound 175)

By following the procedure given in Example 66 and replacing 24-(trans-1-hexen-1-yl)-fusidic acid pivaloyloxymethyl ester (306) with 24-(4-cyanophenyl)-fusidic acid pivaloyloxymethyl ester (315) the title compound (175) was obtained. ¹³C NMR, (CDCl₃): 173.4, 170.6, 151.6, 148.9, 132.9, 131.9, 130.4, 130.2, 129.2, 119.0, 109.8, 74.3, 71.4, 68.2, 49.1, 48.7, 44.4, 39.4, 39.0, 37.1, 36.3, 36.1, 35.0, 34.4, 32.6, 30.3, 30.0, 27.6, 24.3, 22.6, 22.2, 20.7, 20.2, 18.0, 15.9.

### Example 76

### 24-(3-Biphenyl)-fusidic acid (Compound 176)

By following the procedure given in Example 66 and replacing 24-(trans-1-hexen-1-yl)-fusidic acid pivaloyloxymethyl ester (306) with 24-(3-biphenyl)-fusidic acid pivaloyloxymethyl ester (316) the title compound (176) was obtained. ¹³C NMR, (CDCl₃): 173.8, 170.5, 152.2, 144.4, 140.9, 140.7, 134.0, 129.1, 128.9, 128.7, 128.6, 128.5, 128.0, 127.4, 126.9, 124.5, 74.4, 71.5, 67.9, 48.8, 48.6, 44.4, 39.3, 39.0, 36.8, 36.2, 36.0, 35.5, 34.9, 32.2, 29.9, 27.6, 23.9, 22.6, 22.2, 20.8, 20.6, 20.0, 17.9, 15.9.

### Example 77

### 24-(4-(Trifluoromethyl)phenyl)-fusidic acid (Compound 177)

By following the procedure given in Example 66 and replacing 24-(trans-1-hexen-1-yl)-fusidic acid pivaloyloxymethyl ester (306) with 24-(4-(trifluoromethyl)phenyl)-fusidic acid pivaloyloxymethyl ester (317) the title compound (177) was obtained. ¹³C NMR, (CDCl₃): 173.5, 170.6, 152.3, 147.8, 133.0, 129.8, 129.7, 129.0, 125.0, 74.3, 71.4, 67.9, 49.0, 48.6, 44.4, 39.4, 39.0, 37.0, 36.2, 36.1, 35.0, 34.9, 32.5, 30.0, 27.5, 24.2, 22.5, 22.1, 20.6, 20.0, 18.0, 15.9.

### Example 78

### 24-(4-Methoxyphenyl)-fusidic acid (Compound 178)

By following the procedure given in Example 66 and replacing 24-(trans-1-hexen-1-yl)-fusidic acid pivaloyloxymethyl ester (306) with 24-(4-(methoxyphenyl)-fusidic acid pivaloyloxymethyl ester (318) the title compound (178) was obtained. ¹³C NMR, (CDCl₃): 173.6, 170.7, 157.9, 151.3, 136.4, 133.5, 130.6, 129.7, 128.2, 113.2, 74.4, 71.5, 68.0, 55.2, 49.0, 48.6, 44.2, 39.4, 39.0, 36.9, 36.3, 36.0, 35.3, 35.1, 32.1, 30.0, 27.4, 23.8, 22.8, 22.1, 20.8, 20.7, 20.0, 17.9, 15.9.

### Example 79

### 24-(3—Cyanophenyl)-fusidic acid (Compound 179)

By following the procedure given in Example 66 and replacing 24-(trans-1-hexen-1-yl)-fusidic acid pivaloyloxymethyl ester (306) with 24-(3-cyanophenyl)-fusidic acid pivaloyloxymethyl ester (319) the title compound (179) was obtained. ¹³C NMR, (CDCl₃): 173.2, 170.5, 152.3, 144.9, 134.0, 132.9, 132.2, 130.6, 129.7, 128.9, 128.8, 119.0, 112.1, 74.3, 71.4, 68.1, 49.1, 48.7, 44.5, 39.4, 39.0, 37.1, 36.3, 36.1, 35.2, 34.4, 32.5, 30.3, 30.0, 27.5, 24.3, 22.6, 22.1, 20.7, 20.2, 18.0, 15.9.

### Example 80

### 24-(2-Methoxyphenyl)-fusidic acid (Compound 180)

By following the procedure given in Example 66 and replacing 24-(trans-1-hexen-1-yl)-fusidic acid pivaloyloxymethyl ester (306) with 24-(2-methoxyphenyl)-fusidic acid pivaloyloxymethyl ester (320) the title compound (180) was obtained. ¹H NMR (CDCl₃): 7.23 (m, 1H), 6.95 (m, 3H), 5.83 (d, 1H), 3.81 (bs, 1H), 3.79 (s, 3H), 3.74 (bs, 1H), 2.81 (dd, 1H), 2.65-0.95 (m, 22H), 1.95 (s, 3H), 1.84 (bs, 3H), 1.50 (bs, 3H) 1.27 (s, 3H), 0.92 (bs, 3H), 0.92 (d, 3H), 0.81 (bs,3H).

### Example 81

### 24-(3-Nitrophenyl)-fusidic acid (Compound 181)

By following the procedure given in Example 66 and replacing 24-(trans-1-hexen-1-yl)-fusidic acid pivaloyloxymethyl ester (306) with 24-(3-nitrophenyl)-fusidic acid pivaloyloxymethyl ester (321) the title compound (181) was obtained. ¹³C NMR, (CDCl₃): 173.1, 170.5, 152.5, 148.2, 145.3, 135.8, 132.1, 130.9, 128.9, 128.6, 124.1, 121.1, 74.3, 71.4, 68.1, 49.1, 48.6, 44.5, 39.4, 38.9, 37.0, 36.2, 36.1, 35.2, 34.4, 32.5, 30.2, 30.0, 27.5, 24.2, 22.6, 22.2, 20.6, 20.3, 18.0, 15.9.

### Example 82

### 24-(3-Bromophenyl)-fusidic acid (Compound 182)

By following the procedure given in Example 66 and replacing 24-(trans-1-hexen-1-yl)-fusidic acid pivaloyloxymethyl ester (306) with 24-(3-bromophenyl)-fusidic acid pivaloyloxymethyl ester (322) the title compound (182) was obtained. ¹³C NMR, (CDCl₃): 173.8, 170.7, 151.7, 146.1, 132.9, 132.2, 129.6, 129.5, 129.3, 129.0, 128.4, 122.0, 74.3, 71.5, 68.1, 49.0, 48.6, 44.4, 39.4, 39.0, 36.9, 36.3, 36.1, 35.5, 34.8, 32.2, 30.0, 27.6, 24.0, 22.8, 22.1, 20.8, 20.7, 20.0, 17.9, 15.9.

### Example 83

### 24-(4-(Methylthio)phenyl)-fusidic acid (Compound 183)

By following the procedure given in Example 66 and replacing 24-(trans-1-hexen-1-yl)-fusidic acid pivaloyloxymethyl ester (306) with 24-(4-(methylthio)phenyl)-fusidic acid pivaloyloxymethyl ester (323) the title compound (183) was obtained. ¹³C NMR, (CDCl₃): (CDCl3) 173.6, 170.7, 151.5, 140.9, 135.9, 133.5, 130.1, 129.5, 128.5, 126.0, 74.4, 71.5, 68.0, 49.0, 48.6, 44.3, 39.4, 39.0, 36.9, 36.3, 36.1, 35.3, 35.0, 32.2, 30.0, 27.4, 23.9, 22.8, 22.1, 20.8, 20.7, 19.9, 17.9, 15.9, 15.7.

### Example 84

### 24-(2-Naphtyl)-fusidic acid (Compound 184)

By following the procedure given in Example 66 and replacing 24-(trans-1-hexen-1-yl)-fusidic acid pivaloyloxymethyl ester (306) with 24-(2-naphtyl)-fusidic acid pivaloyloxymethyl ester (324) the title compound (184) was obtained. ¹³C NMR, (CDCl₃): 174.4, 170.8, 151.6, 141.7, 134.0, 133.4, 131.9, 129.6, 128.7, 128.6, 127.8, 127.6, 127.4, 126.2, 125.6, 74.3, 71.5, 67.5, 48.7, 48.4, 44.2, 39.3, 38.9, 36.6, 36.3, 35.8, 35.2, 35.1, 31.9, 29.9, 29.7, 27.4, 23.6, 22.7, 22.2, 20.8, 20.6, 19.9, 17.8, 15.9.

### Example 85

### 24-(3,5-bis-(Trifluoromethyl)phenyl)-fusidic acid (Compound 185)

By following the procedure given in Example 66 and replacing 24-(trans-1-hexen-1-yl)-fusidic acid pivaloyloxymethyl ester (306) with 24-(3,5-bis-(Trifluoromethyl)phenyl)-fusidic acid pivaloyloxymethyl ester (325) the title compound (185) was obtained. ¹³C NMR, (CDCl₃): 173.1, 170.5, 152.9, 145.9, 131.7, 131.4, 129.5, 128.4, 125.3, 121.7, 120.0, 74.3, 71.4, 67.9, 49.0, 48.6, 44.6, 39.4, 38.9, 37.0, 36.2, 36.1, 35.3, 34.6, 32.5, 30.2, 30.0, 27.6, 24.2, 22.4, 22.1, 20.6, 20.2, 18.0, 15.9.

### Example 86

### 24-(3,4-Dimethoxyphenyl)-fusidic acid (Compound 186)

By following the procedure given in Example 66 and replacing 24-(trans-1-hexen-1-yl)-fusidic acid pivaloyloxymethyl ester (306) with 24-(3,4-dimethoxyphenyl)-fusidic acid pivaloyloxymethyl ester (326) the title compound (186) was obtained. ¹³C NMR, (CDCl₃): 173.4, 170.5, 152.2, 148.5, 147.3, 136.6, 133.7, 129.1, 128.2, 121.7, 113.1, 110.6, 74.4, 71.5, 68.0, 56.1, 55.9, 49.0, 48.6, 44.4, 39.4, 39.0, 36.9, 36.3, 36.1, 35.2, 35.0, 32.2, 30.0, 27.5, 23.9, 22.7, 22.2, 20.8, 20.6, 20.0, 17.9, 15.9.

### Example 87

### 24-(3,5-Dibromophenyl)-fusidic acid (Compound 187)

By following the procedure given in Example 66 and replacing 24-(trans-1-hexen-1-yl)-fusidic acid pivaloyloxymethyl ester (306) with 24-(3,5-dibromophenyl)-fusidic acid pivaloyloxymethyl ester (327) the title compound (187) was obtained. ¹³C NMR, (CDCl₃): 173.3, 170.5, 152.9, 147.4, 131.8, 131.5, 131.2, 130.7, 128.5, 122.4, 74.4, 71.5, 68.1, 49.1, 48.6, 44.6, 39.4, 39.0, 37.0, 36.2, 36.1, 35.6, 34.6, 32.3, 30.2, 30.0, 27.6, 24.1, 22.8, 22.2, 20.8, 20.7, 20.1, 18.0, 15.9.

### Example 88

### 24-Bromofusidic acid, cholin salt (Compound 188)

A solution of cholin hydroxide in methanol (45%, 0.4 ml, 0.18 g, 1.5 mmol) was gradually added, with stirring, to a solution of 24-Bromofusidic acid (108) (893 mg, 1.5 mmol) in ethanol (10 ml). The resulting solution was concentrated under reduced pressure, and the residue was crystallized from ether. The title compound (188) was collected by filtration. ¹³C NMR, (CD₃OD): 179.1, 173.3, 138.5, 138.3, 131.3, 122.6, 76.0, 72.5, 69.1, 68.9, 57.1, 54.7, 50.8, 50.0, 43.7, 40.7, 40.3, 38.5, 38.3, 37.8, 37.5, 36.9, 33.0, 31.1, 31.0, 30.3, 25.4, 23.8, 22.5, 21.1, 20.5, 17.9, 16.5.

### Example 89

### 24-Bromofusidic acid, L-arginine salt (Compound 189)

By following the procedure given in Example 88 and replacing cholin hydroxide with L-arginine (261 mg, 1.5 mmol; in water (10 ml)) the title compound (189) was obtained (from ethyl acetate) as an amorphous powder. ¹³C NMR, (CD₃OD): 179.2, 174.7, 173.3, 158.9, 139.9, 137.6, 131.5, 122.4, 75.9, 72.5, 68.8, 55.6, 50.8, 50.0, 43.9, 41.9, 40.7, 40.2, 38.6, 38.3, 37.9, 37.5, 36.9, 33.0, 31.1, 31.0, 30.1, 29.5, 25.8, 25.4, 23.8, 22.5, 21.1, 20.5, 17.9, 16.5.

### Example 90

### 24-Bromofusidic acid, 2-(dimethylamino)-ethanol salt (Compound 190)

By following the procedure given in Example 88 and replacing cholin hydroxide with 2-(dimethylamino)-ethanol (151 μl, 134 mg, 1.5 mmol) the title compound (190) was obtained (from ether) as crystals. ¹³C NMR, (CD₃OD): 178.1, 173.1, 141.6, 136.4, 131.6, 122.3, 75.9, 72.5, 68.8, 66.9, 60.9, 57.5, 50.8, 50.0, 44.1, 40.7, 40.2, 38.7, 38.3, 37.9, 37.5, 36.9, 33.0, 31.1, 31.0, 30.0, 25.4, 23.8, 22.5, 21.1, 20.5, 17.9, 16.5, 15.5.

### Example 91

### 24-Bromofusidic acid, 4-(2-hydroxyethyl)-morpholin salt (Compound 191)

By following the procedure given in Example 88 and replacing cholin hydroxide with 4-(2-hydroxyethyl)-morpholin (184 μl, 197 mg, 1.5 mmol) the title compound (191) was obtained (from di-isopropylether) as an amorphous powder. ¹³C NMR, (CD₃OD): 175.0, 172.7, 147.7, 132.4, 132.2, 121.7, 75.8, 72.5, 68.6, 66.9, 61.3, 58.7, 54.6, 50.7, 49.9, 44.9, 40.7, 40.1, 38.8, 38.2, 37.8, 37.4, 36.8, 32.9, 31.1, 31.0, 29.3, 25.5, 23.9, 23.1, 22.4, 20.8, 20.6, 18.0, 16.5.

### Example 92

### 24-Bromofusidic acid, L-lysine salt (Compound 192)

By following the procedure given in Example 88 and replacing cholin hydroxide with L-lysine (219 mg, 1.5 mmol; in water (5 ml)) the title compound (192) was obtained (from ethyl acetate) as crystals. ¹³C NMR, (CD₃OD): 179.1, 175.1, 173.2, 139.4, 137.9, 131.4, 122.5, 75.9, 72.5, 68.8, 55.9, 50.8, 50.0, 43.9, 40.7, 40.4, 40.3, 38.6, 38.3, 37.9, 37.5, 36.9, 33.0, 32.0, 31.1, 31.0, 30.2, 28.5, 25.4, 23.8, 23.2, 22.5, 21.2, 20.6, 17.9, 16.5.

### Example 93

### 24-Bromofusidic acid, N-(2-hydroxyethyl)-pyrrolidine salt (Compound 193)

By following the procedure given in Example 88 and replacing cholin hydroxide with N-(2-hydroxyethyl)-pyrrolidine (177 μl, 173 mg, 1.5 mmol) the title compound (193) was obtained (from di-isopropylether) as crystals. ¹³C NMR, (CD₃OD): 178.5, 173.1, 140.7, 137.0, 131.5, 122.4, 75.9, 72.5, 70.1, 68.8, 58.3, 55.2, 50.8, 50.0, 44.0, 40.7, 40.2, 38.6, 38.3, 37.8, 37.5, 36.9, 33.0, 31.1, 31.0, 30.1, 25.4, 24.0, 23.8, 23.1, 22.5, 21.1, 20.5, 17.9, 16.5.

### Example 94

### 24-Bromofusidic acid, ethanolamine salt ( Compound 194)

By following the procedure given in Example 88 and replacing cholin hydroxide with ethanolamine (90 μl, 92 mg, 1.5 mmol) the title compound (194) was obtained (from ether) as an amorphous powder. ¹³C NMR, (CD₃OD): 178.9, 173.3, 139.7, 137.6, 131.4, 122.5, 75.9, 72.5, 68.8, 59.4, 50.8, 50.0, 43.9, 43.0, 40.7, 40.2, 38.6,.38.3, 37.8, 37.5, 36.9, 33.0, 31.1, 31.0, 30.1, 25.4, 23.8, 22.5, 21.1, 20.5, 17.9, 16.5.

### Example 95

### 24-Bromofusidic acid, potassium salt (Compound 195)

An aq. solution of potassium hydroxide (0.82 M; 1.8 ml, 1.5 mmol) was gradually added to a solution of 24-Bromofusidic acid (108) (893 mg, 1.5 mmol) in ethanol (10 ml) and water (2.5 ml). The pH of the solution was monitored by means of a pH-meter. At approx. pH 7 more water (7.5 ml) was added. The resulting solution (end-pH 10) was concentrated under reduced pressure, and the residue was crystallized from acetone. The title compound (195) was collected by filtration. ¹³C NMR, (CD₃OD): 179.2, 173.4, 138.6, 138.4, 131.3, 122.6, 75.9, 72.5, 68.9, 50.8, 50.0, 43.7, 40.7, 40.3, 38.5, 38.3, 37.8, 37.5, 36.9, 33.0, 31.1, 31.0, 30.3, 25.4, 23.8, 22.5, 21.2, 20.5, 17.9, 16.5, 8.1.

### Example 96

### 24-Bromofusidic acid, tetrabutylammonium salt (Compound 196)

By following the procedure given in Example 95 and replacing potassium hydroxide with aq. tetrabutylammonium hydroxide (ca.25%, 1.8 ml, 1.5 mmol; end-pH=9), the title compound (196) was obtained (from ether) as crystals. ¹³C NMR, (CD₃OD): 178.3, 173.2, 140.3, 137.1, 131.4, 122.5, 76.0, 72.5, 68.8, 59.5, 50.8, 50.0, 43.9, 40.7, 40.2, 38.6, 38.3, 37.8, 37.5, 36.8, 32.9, 31.1, 31.0, 30.1, 25.4, 24.8, 23.8, 22.5, 21.1, 20.7, 20.5, 17.9, 16.5, 14.0.

### Example 97

### 24-Bromofusidic acid, benzyltrimethylammonium salt (Compound 197)

By following the procedure given in Example 95 and replacing potassium hydroxide with benzyltrimethylammonium hydroxide (ca.40% in methanol; end-pH=9), the title compound (197) was obtained (from acetone) as crystals. ¹H NMR, (CD₃OD): 7.55 (m, 5H), 5.74 (d, 1H), 4.53 (s, 2H), 4.30 (br, 1H), 3.64 (br, 1H), 3.10 (s, 9H), 3.00 (d, 1H), 2.8-2.0 (m, 8H), 1.99 (s, 3H), 1.91-1.40 (m, 7H), 1.83 (s,3H), 1.80 (s, 3H), 1.38 (s, 3H), 1.2-1.0 (m, 3H), 0.99 (s, 3H), 0.95 (s, 3H), 0.89 (d, 3H).

### Example 98

### 24-Bromofusidic acid, cetyltrimethylammonium salt (Compound 198)

By following the procedure given in Example 95 and replacing potassium hydroxide with cetyltrimethylammonium hydroxide (ca.10% in water; end-pH=10), the title compound (198) was obtained (from methyl ethyl ketone) as crystals. ¹³C NMR, (CD₃OD): 179.2, 173.3, 138.4, 138.3, 131.2, 122.6, 76.0, 72.5, 68.9, 67.9, 53.5, 50.8, 50.0, 43.7, 40.7, 40.3, 38.5, 38.3, 37.8, 37.5, 36.8, 33.1, 32.9, 31.1, 31.0, 30.8, 30.7, 30.6, 30.5, 30.3, 27.4, 25.4, 24.0, 23.9, 23.8, 22.5, 21.1, 20.5, 17.9, 16.5, 14.5.

### Example 99

### 24-Bromofusidic acid, tetramethylammonium salt (Compound 199)

By following the procedure given in Example 95 and replacing potassium hydroxide with tetramethylammonium hydroxide (ca.10% in water; end-pH=10), the title compound (199) was obtained (from acetone/ether) as crystals. ¹³C NMR, (CD₃OD): 179.2, 173.3, 138.4, 138.3, 131.2, 122.6, 76.0, 72.5, 68.9, 55.9, 50.8, 50.0, 43.7, 40.7, 40.3, 38.5, 38.3, 37.8, 37.5, 36.8, 32.9, 31.1, 31.0, 30.3, 25.4, 23.8, 23.8, 22.5, 21.1, 20.5, 17.9, 16.5.

### Example 100

### 24-Bromofusidic acid, tetrapropylammonium salt (Compound 300)

By following the procedure given in Example 95 and replacing potassium hydroxide with tetrapropylammonium hydroxide (ca.10% in water; end-pH=9.5), the title compound (300) was obtained (from acetone/ether) as crystals. ¹³C NMR, (CD₃OD): 179.2, 173.3, 138.4, 138.3, 131.2, 122.7, 76.0, 72.5, 68.9, 61.3, 50.8, 50.0, 43.7, 40.7, 40.3, 38.5, 38.3, 37.8, 37.5, 36.9, 33.0, 31.1, 31.0, 30.3, 25.4, 23.8, 23.8, 22.5, 21.1, 20.5, 17.9, 16.5, 16.4, 10.9.

### Example 101

### 24-Bromofusidic acid, tris(hydroxymethyl)aminomethane salt (Compound 301)

By following the procedure given in Example 88 and replacing potassium hydroxide with tris(hydroxymethyl)aminomethan (82 mg, 1.5 mmol), dissolved in ethanol (12 ml) and water (8 ml), the title compound (301) was obtained (from ether) as an amorphous powder. ¹³C NMR, (CD₃OD): 178.8, 173.3, 140.1, 137.4, 131.4, 122.4, 76.0, 72.5, 70.1, 68.8, 61.8, 50.8, 50.0, 43.9, 40.7, 40.2, 38.6, 38.3, 37.8, 37.5, 36.9, 32.9, 31.1, 31.0, 30.1, 25.4, 23.8, 23.1, 22.5, 21.1, 20.5, 17.9, 16.5.

### Example 102

### 24-Bromofusidic acid, N-methyl-D-glucamine salt (Compound 302)

By following the procedure given in Example 88 and replacing potassium hydroxide with N-methyl-D-glucamine (293 mg, 1.5 mmol), dissolved in ethanol (5 ml) and water (5 ml), the title compound (302) was obtained (from ether) as an amorphous powder.

¹³C NMR, (CD₃OD): 179.2, 173.3, 139.5, 137.8, 131.4, 122.5, 75.9, 73.0, 72.5, 72.2, 70.1, 68.8, 64.8, 61.6, 53.0, 50.8, 50.0, 43.9, 40.7, 40.3, 38.6, 38.3, 37.8, 37.5, 36.9, 34.2, 33.0, 31.0, 30.2, 25.4, 23.8, 22.5, 21.2, 20.9, 20.6, 17.9, 16.5, 14.5.

### Example 103

### 24-Bromofusidic acid, silver salt (Compound 303)

A solution of 24-bromo fusidic acid sodium salt (109) (926 mg, 1.5 mmol) in water (10 ml) was added to a solution of silver acetate (250 mg, 1.5 mmol) In water (30 ml). Ethanol (25 ml) was added, and after one hour the fine grained precipitate was collected by filtration (through a small pore glass filter), washed with water and dried in vacuo over silica gel for several days, in the dark. The silver salt (303) was a light grey, amorphous powder. ¹H NMR, (DMSO): 5.68 (d, 1H), 4.14 (br, 1H), 4.00 (m, 2H), 3.51 (br, 1H), 2.9-0.93 (m, 20H), 1.90 (s, 3H), 1.79 (s, 3H), 1.78 (s, 3H), 1.27 (s,3H), 0.89 (s, 3H), 0.82 (s, 3H), 0.79 (d, 3H).

### Example 104

### 24-Bromofusidic acid, benzethonium salt (Compound 304)

A solution of benzethonium chloride (672 mg, 1.5 mmol) in water (5 ml) was added to a solution of 24-bromo fusidic acid sodium salt (109) (926 mg, 1.5 mmol) in water (10 ml), whereby a sticky precipitate of benzethonium salt was formed. The aqueous phase was decanted and the precipitate was washed with water by decantation. The precipitate was dissolved in acetone, concentrated, and evaporated with ethyl acetate (three times), in order to remove water. Treatment with ether left the salt as a sticky mass which was filtered off and left in the air overnight to yield the title compound (304) as a brittle, amorphous powder. ¹³C NMR, (CD₃OD): 179.0, 173.3, 157.8, 143.6, 138.7, 138.2, 134.4, 132.0, 131.3, 130.4, 128.9, 128.4, 122.6, 114.8, 76.0, 72.5, 71.0, 70.4, 68.9, 68.3, 66.9, 65.9, 64.7, 58.0, 51.4, 50.8, 50.0, 43.7, 40.7, 40.3, 38.9, 38.5, 38.3, 37.8, 37.5, 36.8, 33.1, 32.9, 32.4, 32.3, 31.1, 31.0, 30.3, 25.4, 23.8, 23.8, 22.5, 21.1, 20.5, 17.9, 16.5, 15.5.

### Example 105

### 24-Bromofusidic acid, triethanolamine salt (Compound 305)

By following the procedure given in Example 88 and replacing cholin hydroxide with triethanolamine (209 μl, 234 mg, 1.5 mmol; neat) the title compound (305) was obtained (from ethanol/ether) as a crystalline powder. ¹³C NMR, (CD₃OD): 176.4, 172.9, 144.7, 134.3, 131.9, 122.0, 75.8, 72.5, 68.7, 58.7, 57.6, 50.8, 50.0, 44.5, 40.7, 40.1, 38.7, 38.2, 37.8, 37.5, 36.8, 32.9, 31.0, 29.6, 25.5, 23.8, 22.4, 20.9, 20.5, 18.0, 16.5.

### Example 106

### 24-(trans-1-Hexen-1-yl)-fusidic acid pivaloyloxymethylester (Compound 306)

By following the procedure given in Example 36 and replacing phenylboronic acid with trans-1-Hexen-1-ylboronic acid the title compound (306) was obtained. ¹³C NMR, (CDCl₃): 177.0, 170.3, 168.4, 149.9, 129.8, 129.7, 129.6, 128.4, 127.8, 80.0, 74.4, 71.4, 68.2, 49.2, 48.7, 44.2, 39.4, 39.0, 38.8, 37.1, 36.2, 36.2, 35.7, 33.2, 32.5, 32.1, 30.3, 30.0, 27.8, 27.6, 26.9, 24.3, 22.7, 22.4, 21.5, 20.8, 20.7, 20.4, 17.9, 15.9, 14.0.

### Example 107

### with 24-(Trans-1-Buten-3,3-dimethyl-1-yl)-fusidic acid pivaloyloxymethyl ester (Compound 307)

By following the procedure given in Example 36 and replacing phenylboronic acid with trans-1-buten-3,3-dimethyl-1-ylboronic acid the title compound (307) was obtained.

¹³C NMR, (CDCl₃): 177.0, 170.3, 168.5, 149.9, 139.2, 130.1, 129.7, 129.5, 122.5, 80.0, 74.4, 71.4, 68.2, 49.2, 48.7, 44.2, 39.4, 39.0, 38.8, 37.1, 36.2, 36.2, 35.6, 33.4, 32.5, 30.3, 30.0, 29.0, 27.8, 27.7, 26.9, 24.3, 22.7, 21.6, 20.8, 20.7, 20.4, 17.8, 15.9.

### Example 108

### 24-(trans-1-Nonen-1-yl)-fusidic acid pivaloyloxymethyl ester (Compound 308)

By following the procedure given in Example 36 and replacing phenylboronic acid with trans-1-Nonen-1-ylboronic acid the title compound (308) was obtained. ¹³C NMR, (CDCl₃): 177.0, 170.3, 168.4, 149.9, 129.8, 129.7, 129.6, 128.5, 127.8, 80.0, 74.4, 71.4, 68.2, 49.2, 48.7, 44.2, 39.4, 39.0, 38.8, 37.1, 36.3, 36.1, 35.7, 33.6, 32.5, 31.9, 30.3, 30.0, 30.0, 29.4, 29.3, 27.8, 27.6, 26.9, 24.3, 22.7, 22.7, 21.5, 20.8, 20.7, 20.4, 17.9, 15.9, 14.1.

### Example 109

### 24-(trans-5—Chloro-1-penten-1-yl)-fusidic acid pivaloyloxymethyl ester (Compound 309)

By following the procedure given in Example 36 and replacing phenylboronic acid with trans-5-chloro-1-penten-1-ylboronic acid the title compound (309) was obtained. ¹³C NMR, (CDCl₃): 177.0, 170.2, 168.4, 149.9, 130.7, 129.6, 129.5, 129.3, 125.9, 80.0, 74.4, 71.4, 68.2, 49.2, 48.7, 44.6, 44.2, 39.4, 39.0, 38.8, 37.1, 36.3, 36.1, 35.7, 32.6, 30.5, 30.3, 30.0, 27.8, 27.6, 26.9, 24.3, 22.6, 21.5, 20.8, 20.7, 20.4, 17.9, 15.9.

### Example 110

### 24-(trans-2-Phenyl-1-vinyl)-fusidic acid pivaloyloxymethyl ester (Compound 310)

By following the procedure given in Example 36 and replacing phenylboronic acid with trans-2-phenyl-1-vinylboronic acid the title compound (310) was obtained. ¹³C NMR, (CDCl₃): 177.0, 170.2, 168.4, 150.4, 138.4, 133.6, 130.3, 129.3, 128.6, 127.1, 126.9, 126.3, 126.1, 80.1, 74.4, 71.4, 68.1, 49.2, 48.7, 44.3, 39.4, 39.0, 38.8, 37.0, 36.2, 36.1, 35.9, 32.4, 30.2, 29.9, 27.9, 27.6, 26.9, 24.1, 22.7, 21.9, 20.8, 20.8, 20.7, 17.8, 15.9.

### Example 111

### 24-(2-Phenyl-1-ethyl)-fusidic acid pivaloyloxymethyl ester (Compound 311)

24-(trans-2-Phenyl-1-vinyl)-fusidic acid pivaloyloxymethyl ester (310) (230 mg, 0.3 mmol) was dissolved in ethanol (5 ml) and palladium-on-carbon (25 mg, 5%) was added. The flask was evacuated, and a balloon, containing hydrogen gas, was fitted to the flask which was then left overnight with magnetic stirring. The catalyst was filtered off, through filter aid, and the filtrate was concentrated. The crude product was purified by FCC (eluant: pet.ether:EtOAc, 90:10 to 50:50) to yield the pure title compound (311). ¹³C NMR, (CD₃OD): 178.2, 172.1, 169.7, 152.1, 152.1, 144.1, 143.9, 130.9, 130.8, 129.4, 129.3, 126.6, 81.0, 75.6, 72.3, 68.4, 50.6, 45.5, 44.8, 44.4, 40.6, 40.0, 39.7, 38.1, 37.8, 37.4, 37.3, 36.7, 35.0, 34.8, 33.6, 33.3, 32.8, 31.9, 31.0, 30.2, 28.2, 27.3, 23.9, 23.0, 22.3, 20.9, 19.6, 19.5, 18.2, 16.5.

### Example 112

### 24-(4-n-Propylphenyl)-fusidic acid pivaloyloxymethyl ester (Compound 312)

By following the procedure given in Example 36 and replacing phenylboronic acid with 4-n-propylphenylboronic acid the title compound (312) was obtained. ¹³C NMR, (CDCl₃): 177.0, 170.2, 167.6, 152.5, 141.4, 140.5, 133.8, 129.5, 129.0, 128.2, 127.8, 80.0, 74.4, 71.4, 67.9, 48.9, 48.7, 44.4, 39.4, 39.0, 38.8, 37.7, 36.9, 36.3, 36.1, 35.5, 35.0, 32.2, 30.1, 30.0, 27.4, 26.9, 24.7, 23.8, 22.7, 22.1, 20.9, 20.8, 19.9, 18.0, 15.9, 14.0.

### Example 113

### 24-(4-Vinylphenyl)-fusidic acid pivaloyloxymethyl ester (Compound 313)

By following the procedure given in Example 36 and replacing phenylboronic acid with 4-vinylphenylboronic acid the title compound (313) was obtained. ¹³C NMR, (CDCl₃): 177.0, 170.2, 167.7, 152.6, 143.8, 136.4, 135.4, 133.7, 129.9, 128.8, 128.5, 125.8, 113.6, 80.0, 74.3, 71.4, 67.8, 48.9, 48.6, 44.4, 39.4, 39.0, 38.8, 36.9, 36.3, 36.0, 35.3, 35.0, 32.2, 30.0, 27.3, 26.9, 23.9, 22.7, 22.1, 20.8, 20.8, 19.9, 17.9, 15.9.

### Example 114

### 24-(4-tert-Butylphenyl)-fusidic acid pivaloyloxymethyl ester (Compound 314)

By following the procedure given in Example 36 and replacing phenylboronic acid with 4-tert-butylphenylboronic acid the title compound (314) was obtained. ¹³C NMR, (CDCl₃): 177.0, 170.2, 167.6, 152.5, 149.0, 140.9, 133.7, 129.1, 129.0, 128.3, 124.6, 80.0, 74.4, 71.4, 67.9, 48.9, 48.8, 44.3, 39.4, 39.0, 38.8, 36.9, 36.2, 36.1, 35.5, 34.8, 34.5, 32.3, 31.5, 30.3, 30.0, 27.5, 26.9, 23.9, 22.6, 22.1, 20.9, 20.7, 20.0, 18.1, 15.9.

### Example 115

### 24-(4-Cyanophenyl)-fusidic acid pivaloyloxymethyl ester (Compound 315)

By following the procedure given in Example 36 and replacing phenylboronic acid with 4-cyanophenylboronic acid the title compound (315) was obtained. ¹³C NMR, (CDCl₃): 177.0, 170.1, 167.7, 152.2, 148.9, 132.8, 131.9, 130.5, 130.2, 128.5, 119.0, 109.9, 80.0, 74.2, 71.3, 68.1, 49.1, 48.6, 44.5, 39.4, 39.0, 38.8, 37.1, 36.3, 36.0, 34.9, 34.4, 32.6, 30.3, 30.0, 27.5, 26.9, 24.3, 22.6, 22.2, 20.8, 20.6, 20.2, 18.0, 15.9.

### Example 116

### 24-(3-Biphenyl)-fusidic acid pivaloyloxymethyl ester (Compound 316)

By following the procedure given in Example 36 and replacing phenylboronic acid with 3-biphenylboronic acid the title compound (316) was obtained. ¹³C NMR, (CDCl₃): 177.0, 170.2, 167.8, 152.3, 144.4, 140.9, 140.7, 133.9, 128.9, 128.8, 128.6, 128.6, 128.5, 128.0, 127.5, 126.9, 124.5, 80.0, 74.3, 71.4, 67.8, 48.8, 48.6, 44.4, 39.3, 39.0, 38.8, 36.8, 36.2, 36.1, 35.5, 34.8, 32.2, 30.0, 27.6, 26.9, 23.9, 22.5, 22.2, 20.8, 20.7, 20.0, 18.0, 15.9.

### Example 117

### 24-(4-(Trifluoromethyl)phenyl)-fusidic acid pivaloyloxymethyl ester (Compound 317)

By following the procedure given in Example 36 and replacing phenylboronic acid with 4-(trifluoromethyl)phenylboronic acid the title compound (317) was obtained. ¹³C NMR, (CDCl₃): 177.0, 170.2, 167.7, 152.6, 147.8, 133.0, 129.8, 129.7, 128.5, 125.0, 124.9, 80.0, 74.2, 71.4, 67.9, 49.0, 48.6, 44.5, 39.3, 39.0, 38.8, 37.0, 36.2, 36.1, 34.9, 34.8, 32.5, 30.0, 27.4, 26.9, 24.2, 22.4, 22.1, 20.8, 20.6, 20.0, 18.0, 15.9.

### Example 118

### 24-(4-Methoxyphenyl)-fusidic acid pivaloyloxymethyl ester (Compound 318)

By following the procedure given in Example 36 and replacing phenylboronic acid with 4-methoxyphenylboronic acid the title compound (318) was obtained. ¹³C NMR, (CDCl₃): 177.0, 170.2, 167.7, 157.9, 152.2, 136.3, 133.4, 130.6, 129.0, 128.2, 113.2, 80.0, 74.3, 71.4, 67.9, 55.2, 49.0, 48.6, 44.4, 39.4, 39.0, 38.8, 36.9, 36.3, 36.0, 35.3, 35.1, 32.1, 30.0, 27.3, 26.9, 23.8, 22.8, 22.0, 20.8, 20.0, 17.9, 15.9.

### Example 119

### 24-(3-Cyanophenyl)-fusidic acid pivaloyloxymethyl ester (Compound 319)

By following the procedure given in Example 36 and replacing phenylboronic acid with 3-cyanophenylboronic acid the title compound (319) was obtained. ¹³C NMR, (CDCl₃): 177.0, 170.2, 167.7, 152.3, 144.9, 134.0, 132.9, 132.2, 130.6, 129.7, 128.9, 128.5, 119.0, 112.2, 80.0, 74.2, 71.3, 68.1, 49.1, 48.7, 44.5, 39.4, 39.0, 38.8, 37.1, 36.3, 36.1, 35.2, 34.4, 32.6, 30.3, 30.0, 27.5, 26.9, 24.3, 22.5, 22.1, 20.8, 20.6, 20.2, 18.0, 15.9.

### Example 120

### 24-(2-Methoxyphenyl)-fusidic acid pivaloyloxymethyl ester (Compound 320)

By following the procedure given in Example 36 and replacing phenylboronic acid with 2-methoxyphenylboronic acid the title compound (320) was obtained. ¹³C NMR, (CDCl₃): 177.0, 170.2, 167.8, 157.2, 152.2, 132.7, 131.9, 130.9, 130.3, 129.1, 127.5, 120.3, 111.0, 79.9, 74.4, 71.5, 68.0, 55.9, 55.4, 48.9, 48.7, 44.4, 39.4, 39.0, 38.8, 36.8, 36.4, 36.0, 35.4, 34.4, 33.2, 32.1, 30.0, 27.2, 26.9, 23.8, 22.8, 22.0, 20.9, 19.7, 17.9, 15.9.

### Example 121

### 24-(3-Nitrophenyl)-fusidic acid pivaloyloxymethyl ester (Compound 321)

By following the procedure given in Example 36 and replacing phenylboronic acid with 3-nitrophenylphenylboronic acid the title compound (321) was obtained. ¹³C NMR, (CDCl₃): 177.0, 170.2, 167.7, 152.3, 148.3, 145.3, 135.7, 132.1, 130.9, 129.0, 128.4, 124.1, 121.1, 80.0, 74.2, 71.3, 68.1, 49.0, 48.6, 44.5, 39.4, 39.0, 38.8, 37.0, 36.3, 36.1, 35.2, 34.3, 32.6, 30.3, 30.0, 27.6, 26.9, 24.3, 22.5, 22.2, 20.8, 20.6, 20.3, 18.0, 15.9.

### Example 122

### 24-(3-Bromophenyl)-fusidic acid pivaloyloxymethyl ester (Compound 322)

By following the procedure given in Example 36 and replacing phenylboronic acid with 3-bromophenylboronic acid the title compound (322) was obtained. ¹³C NMR, (CDCl₃): 177.0, 170.2, 167.7, 152.5, 146.1, 132.8, 132.2, 129.6, 129.5, 129.0, 128.5, 128.4, 122.0, 80.0, 74.3, 71.4, 68.0, 49.0, 48.6, 44.5, 39.4, 39.0, 38.8, 36.9, 36.3, 36.1, 35.5, 34.8, 32.2, 30.1, 30.0, 27.5, 26.9, 24.0, 22.7.

### Example 123

### 24-(4-(Methylthio)phenyl)-fusidic acid pivaloyloxymethyl ester (Compound 323)

By following the procedure given in Example 36 and replacing phenylboronic acid with 4-(methylthio)phenylboronic acid the title compound (323) was obtained. ¹³C NMR, (CDCl₃): 177.0, 170.2, 167.7, 152.5, 140.8, 135.9, 133.3, 130.1, 128.7, 128.5, 125.9, 79.9, 74.3, 71.4, 67.9, 48.9, 48.6, 44.4, 39.4, 39.0, 38.8, 36.9, 36.3, 36.0, 35.2, 35.0, 32.1, 30.0, 29.9, 27.3, 26.9, 23.9, 22.8, 22.0, 20.8, 20.8, 19.9, 17.9, 15.9, 15.6.

### Example 124

### 24-(2-Naphtyl)-fusidic acid pivaloyloxymethyl ester (Compound 324)

By following the procedure given in Example 36 and replacing phenylboronic acid with 2-naphtylboronic acid the title compound (324) was obtained. ¹³C NMR, (CDCl₃): 177.0, 170.2, 167.7, 152.7, 141.7, 133.9, 133.3, 131.9, 128.7, 128.6, 128.6, 127.8, 127.6, 127.4, 126.2, 125.6, 80.0, 74.3, 71.4, 67.4, 48.6, 48.4, 44.4, 39.3, 39.0, 38.8, 36.6, 36.3, 35.8, 35.2, 35.1, 32.0, 29.9, 29.6, 27.4, 26.9, 23.7, 22.6, 22.2, 20.8, 20.8, 19.9, 17.8, 15.9.

### Example 125

### 24-(3,5-bis-(Trifluoromethyl)phenyl)-fusidic acid pivaloyloxymethyl ester (Compound 325)

By following the procedure given in Example 36 and replacing phenylboronic acid with 3,5-bis-(trifluoromethyl)phenylboronic acid the title compound (325) was obtained. ¹³C NMR, (CDCl₃): 176.9, 170.2, 167.6, 152.7, 145.9, 131.7, 131.7, 131.4, 129.4, 128.2, 123.5, 120.0, 80.1, 74.2, 71.3, 67.9, 60.4, 49.0, 48.6, 44.6, 39.4, 39.0, 38.8, 37.0, 36.2, 36.1, 35.3, 34.6, 32.5, 30.2, 30.0, 27.6, 26.9, 24.3, 22.4, 22.1, 20.8, 20.6, 20.2, 18.0, 15.9, 14.2.

### Example 126

### 24-(3,4-Dimethoxyphenyl)-fusidic acid pivaloyloxymethyl ester (Compound 326)

By following the procedure given in Example 36 and replacing phenylboronic acid with 3,4-dimethoxyphenylboronic acid the title compound (326) was obtained. ¹³C NMR, (CDCl₃): 177.0, 170.2, 167.8, 152.1, 148.5, 147.3, 136.6, 133.7, 128.9, 128.2, 121.7, 113.1, 110.6, 80.0, 74.3, 71.4, 67.9, 56.1, 55.8, 49.0, 48.6, 44.4, 39.4, 39.0, 38.8, 36.9, 36.3, 36.0, 35.2, 35.0, 32.2, 30.0, 27.5, 26.9, 23.9, 22.7, 22.1, 20.8, 20.8, 20.0, 17.9, 15.9, 14.2.

### Example 127

### 24-(3,5-Dibromophenyl)-fusidic acid pivaloyloxymethyl ester (Compound 327)

By following the procedure given in Example 36 and replacing phenylboronic acid with 3,5-dibromophenylboronic acid the title compound (327) was obtained. ¹³C NMR, (CDCl₃): 177.0, 170.2, 167.7, 152.7, 147.4, 131.8, 131.5, 131.2, 130.7, 128.3, 122.5, 80.0, 74.2, 71.4, 68.1, 49.1, 48.6, 44.6, 39.4, 39.0, 38.8, 36.9, 36.3, 36.1, 35.6, 34.6, 32.3, 30.2, 30.0, 27.5, 26.9, 24.1, 22.7, 22.2, 20.8, 20.8, 20.1, 18.0, 15.9.

1. A compound of general formula I 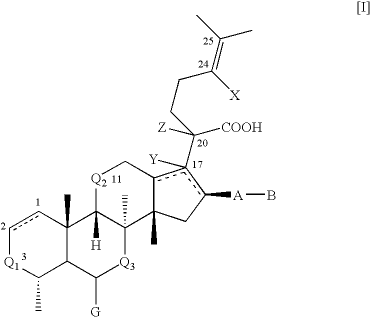
wherein X represents halogen, trifluoromethyl, cyano, azido, alkyl, alkenyl or aryl, wherein said alkyl, alkenyl or aryl are optionally substituted by one or more, same or different substituents selected from the group consisting of alkyl, alkenyl, aryl, alkoxy, nitro, alkylthio, halogen, azido, trifluoromethyl and cyano;
Y and Z both represent hydrogen, or together with the C-17/C-20 bond form a double bond between C-17 and C-20, or together are methylene and form a cyclopropane ring in combination with C-17 and C-20;
A represents a bond, O, S or S(O);
B represents C1-6 alkyl, C2-6 alkenyl, C1-6 acyl, C3-7 cycloalkylcarbonyl or benzoyl, all of which are optionally substituted with one or more substituents selected from the group consisting of halogen, hydroxy, alkoxy, aryl, heteroaryl and azido, or, if A represents a bond, B may also represent hydrogen;
Q1 and Q2 independently represent —CH2—, —C(O)—, —(CHOH)—, —(CHOR)—, —(CHSH)—, —(NH)—, —(CHNH2)— or —(CHW)—, wherein R represents C1-6 alkyl and W represents halogen, cyano, azido or trifluoromethyl;
Q3 represents —CH2—, —C(O)— or —CHOH—;
G represents hydrogen, OH or O—CO—CH3;
two bonds in the pentacyclic ring being depicted with full and dotted lines to indicate that either of the two bonds may be a double bond, in which case Y is absent and Z represents hydrogen; the bond between C-1 and C-2 being either a single or a double bond;
and pharmaceutically acceptable salts and easily hydrolysable esters thereof.

2. A compound according to claim 1 of formula Ia 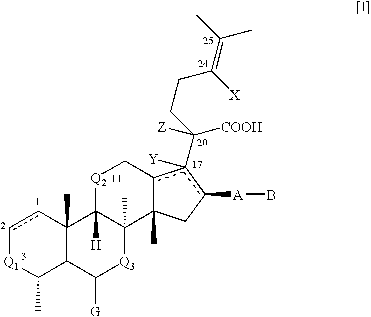
wherein X represents halogen, trifluoromethyl, cyano, azido, C1-7 alkyl, C2-9 alkenyl or aryl,
wherein said C1-6 alkyl, C2-6 alkenyl or aryl are optionally substituted by one or more, same or different substituents selected from the group consisting of C1-7 alkyl, C2-9 alkenyl, aryl, C1-6 alkoxy, nitro, alkylthio, halogen, azido, trifluoromethyl and cyano;
Y and Z both represent hydrogen, or together with the C-17/C-20 bond form a double bond between C-17 and C-20, or together are methylene and form a cyclopropane ring in combination with C-17 and C-20;
A represents a bond, O, S or S(O);
B represents C1-6 alkyl, C2-6 alkenyl, C1-6 acyl, C3-7 cycloalkylcarbonyl or benzoyl, all of which are optionally substituted with one or more substituents selected from the group consisting of halogen, hydroxy, C1-6 alkoxy, aryl, heteroaryl and azido, or, if A represents a bond, B may also represent hydrogen;
Q1 and Q2 independently represent —C(O)—, —(CHOH)—, —(CHSH)—, or —(CHW)—, wherein W represents halogen, cyano, azido or trifluoromethyl;
and pharmaceutically acceptable salts and easily hydrolysable esters thereof.

3. A compound according to claim 1, wherein Y and Z are both hydrogen and wherein the stereochemical configuration is S at both C-17 and C-20.

4. A compound according to claim 1, wherein Y and Z together are methylene and form a cyclopropane ring in combination with C-17 and C-20 and the stereochemical configuration is S at both C-17 and C-20.

5. A compound according to claim 1, wherein Y and Z together with the C-17/C-20 bond form a double bond between C-17 and C-20.

6. A compound according to claim 5, wherein the C-17/C-20 double bond has the same configuration as in fusidic acid.

7. A compound according to claim 1, wherein X represents chloro, bromo, iodo, fluoro, methyl, ethyl, propyl, phenyl, vinyl, propenyl, butenyl, pentenyl, hexenyl, heptenyl, nonenyl, biphenyl or naphthyl, wherein said methyl, ethyl, propyl, phenyl, vinyl, propenyl, butenyl, pentenyl, hexenyl, heptenyl, nonenyl, biphenyl or naphthyl, are optionally substituted by one or more, same or different substituents selected from the group consisting of fluoro, chloro, bromo, phenyl, vinyl, cyano, methoxy, trifluoromethyl, nitro, methylthio, butyl, methyl, ethyl, propyl, butyl, pentyl, hexyl, and heptyl.

8. A compound according to claim 7, wherein X represents fluoro, chloro, bromo, iodo, trifluoromethyl, phenyl, 4-bromophenyl, 4-chlorophenyl, 3,5-difluorophenyl, trans-1-hexen-1-yl, trans-1-buten-3,3-dimethyl-1-yl, trans-1-nonen-1-yl, trans-5-chloro-1-penten-1-yl, trans-2-phenyl-1-vinyl, 2-phenyl-1-ethyl, 4-n-propylphenyl, 4-vinylphenyl, 4-tert-butylphenyl, 4-cyanophenyl, 3-biphenyl, 4-(trifluoromethyl)phenyl, 4-methoxyphenyl, 3-cyanophenyl, 2-methoxyphenyl, 3-nitrophenyl, 3-bromophenyl, 4-(methylthio)phenyl, 2-naphtyl, 3,5-bis-(trifluoromethyl)phenyl, 3,4-dimethoxyphenyl or 3,5-dibromophenyl.

9. A compound according to claim 1, wherein Q1 and Q2 independently represent —C(O)— or —(CHOH)—.

10. A compound according to claim 1, wherein Q1 represents CHF, CHCl, CHBr, CHI or CHN3.

11. A compound according to claim 2, wherein Q1 and Q2 both represent a —(CHOH)— group, or one of Q1 or Q2 represents —(CO)—, or Q1 represents CHF, CHCl, CHBr, CHI or CHN3;
X represents chloro, bromo, iodo, trifluorometyl, azido or cyano;
Z and Y together with the C-17/C-20 bond form a double bond between C-17 and C-20;
A represents O, S or S(O);
B represents a C1-4 alkyl group, optionally substituted with one or more substituents selected from the list consisting of azido, hydroxy, fluoro, chloro and bromo, or B represents a C1-4 acyl group or a benzoyl group, both optionally substituted with one or more halogen atoms.

12. A compound according to claim 11, wherein the halogen atoms with which B is optionally substituted are fluoro or chloro.

13. A compound according to claim 1, wherein A represents O or S(O).

14. A compound according to claim 1, wherein B represents acyl, methyl, ethyl, propyl, butyl, pentyl, propenyl or cyclopentyl, all of which are optionally substituted with one or more substituents selected from the list consisting of methyl, ethyl, propyl, butyl, fluoro, vinyl, hydroxy, phenyl, furfuryl and methoxy.

15. A compound according to claim 14, wherein B is acetyl, isopropyl, ethyl, 2,2,2-trifluoroethyl, vinyl, 1-pentyl, 2-methyl-i-butyl, 3-methyl-1-butyl, cyclopentyl, 2-hydroxyethyl, benzyl, furfaryl, phenyl, 2-fluoroethyl, 2-methoxyethyl, 2,2,2-trichloroethyl, 2-azidoethyl, 2-hydroxyethyl, propyl, tert.-butyl, 1,3-difluoro-isopropyl, propionyl, chloroacetyl or trifluoroacetyl.

16. A compound according to claim 1, wherein Q1 or Q2 or both Q1 and Q2 represent —(COH)— and the stereochemical configuration is a at both C-3 and C-11.

17. A compound according to claim 1, wherein the easily hydrolysable ester is a pivaloyloxymethylester or a acetoxymethylester.

18. A compound according to claim 12, wherein A represents O, B is acetyl, wherein Q1 or Q2 or both Q1 and Q2 represent —(COH)— and the stereochemical configuration is a at both C-3 and C-11, Y and Z together with the C-17/C-20 bond form a double bond between C-17 and C-20 which has the same configuration as in fusidic acid.

19. A pharmaceutically acceptable salt of a compound according to claim 1, wherein the salts are selected from the group consisting of sodium salts, choline salts, L-arginine salts, 2-(dimethylamino)-ethanol salts, 4-(2-hydroxyethyl)-morpholin salts, L-lysine salts, N-(2-hydroxyethyl)-pyrrolidine salts, ethanolamine salts, potassium salts, tetrabutylammonium salts, benzyltrimethylammonium salts, cetyltrimethylammonium salts, tetramethylammonium salts, tetrapropylammonium salts, tris(hydroxymethyl)aminomethane salts, N-methyl-D-glucamine salts, silver salts, benzethonium salts and triethanolamine salts.

20. A compound according to claim 1, which is selected from the group consisting of 24-trifluoromethyl fusidic acid sodium salt (Compound 101),
24-trifluoromethyl fusidic acid pivaloyloxymethyl ester (Compound 102),
24-chloro-fusidic acid (Compound 103),
24-chloro-fusidic acid pivaloyloxymethyl ester (Compound 104),
24-chloro-fusidic acid sodium salt (Compound 105),
24-trifluoromethyl fusidic acid (Compound 106),
24-bromo-fusidic acid acetoxymethyl ester (Compound 107),
24-bromo-fusidic acid (Compound 108),
24-bromo-fusidic acid sodium salt (Compound 109),
24-bromo-fusidic acid pivaloyloxymethyl ester (Compound 110),
24-bromo-16-deacetoxy-16b-thioacetyl-fusidic acid acetoxymethylester (Compound 111),
24-bromo-16-deacetoxy-16b-isopropylthio-fusidic acid (Compound 112),
24-bromo-16-deacetoxy-16b-isopropylsulfinyl-fusidic acid (Compound 113),
24-bromo-16-deacetoxy-16b-thioacetyl-fusidic acid (Compound 114),
24-bromo-17S,20S-dihydrofusidic acid (Compound 115),
24-bromo-16-deacetoxy-16b-ethoxy-fusidic acid (Compound 116),
24-bromo-16-deacetoxy-16b-ethoxy-fusidic acid acetoxymethyl ester (Compound 117),
24-bromo-16-deacetoxy -16b-(2′,2′,2′-trifluoroethoxy)-fusidic acid acetoxymethyl ester (Compound 118),
24-bromo-16-deacetoxy -16b-(2′,2′,2′-trifluoroethoxy)-fusidic acid (Compound 119),
24-bromo-17S,20S-fusidic acid acetoxymethyl ester (Compound 120),
24-bromo-17S,20S-methylene-fusidic acid acetoxymethyl ester (Compound 121),
24-bromo-17S,20S-methylene-fusidic acid (Compound 122),
3-deoxy-3b,24-dibromo-fusidic acid (Compound 123),
3a-azido-24-bromo-3-deoxy-fusidic acid (Compound 124),
24-iodo-fusidic acid (Compound 125),
24-iodo-fusidic acid acetoxymethyl ester (Compound 126),
24-iodo-fusidic acid pivaloyloxymethyl ester (Compound 127),
24-phenyl-fusidic acid pivaloyloxymethylester (Compound 136),
24-phenyl-fusidic acid (Compound 137),
24-(4-bromophenyl)-fusidic acid pivaloyloxymethylester (Compound 138),
24-(4-bromophenyl)-fusidic acid (Compound 139),
24-(4-chlorophenyl)-fusidic acid pivaloyloxymethylester (Compound 140),
24-(4-chlorophenyl)-fusidic acid (Compound 141),
24-(3,5-difluorophenyl)-fusidic acid pivaloyloxymethylester (Compound 142),
24-(3,5-difluorophenyl)-fusidic acid (Compound 143),
3-deoxy-3b,24-dibromo-fusidic acid acetoxymethyl ester (Compound 144),
24-bromo-16-deacetoxy-16b-ethylthio-fusidic acid (Compound 146),
24-bromo-16-deacetoxy-16b-ethylsulfinyl-fusidic acid (Compound 147),
24-bromo-16-deacetoxy-16β-allylthio-fusidic acid (Compound 148),
24-bromo-16-deacetoxy-16b-(1-pentylthio)-fusidic acid (Compound 149),
24-bromo-16-deacetoxy-16b-(1-pentylsulfinyl)-fusidic acid (Compound 150),
24-bromo-16-deacetoxy-16b-(2-methyl-1-butylthio)-fusidic acid (Compound 151),
24-bromo-16-deacetoxy-16b-(2-methyl-1-butylsulfinyl)-fusidic acid (Compound 152),
24-bromo-16-deacetoxy-16b-(3-methyl-1-butylthio)-fusidic acid (Compound 153),
24-bromo-16-deacetoxy-16b-(3-methyl-1-butylsulfinyl)-fusidic acid (Compound 154),
24-bromo-16-deacetoxy-16b-cyclopentylthio-fusidic acid (Compound 155),
24-bromo-16-deacetoxy-16b-(2,2,2-trifluoroethylthio)-fusidic acid (Compound 156),
24-bromo-16-deacetoxy-16b-(2-hydroxyethylthio)-fusidic acid (Compound 157),
24-bromo-16-deacetoxy-16b-benzylthio-fusidic acid (Compound 158),
24-bromo-16-deacetoxy-16b-benzylsulfinyl-fusidic acid (Compound 159),
24-bromo-16-deacetoxy-16b-(2-furylmethylthio)-fusidic acid (Compound 160),
24-bromo-16-deacetoxy-16b-phenylthio-fusidic acid (Compound 161),
24-bromo-16-deacetoxy-16b-benzoylthio-fusidic acid (Compound 162),
24-bromo-16-deacetoxy-16b-isopropoxy-fusidic acid (Compound 163),
24-bromo-16-deacetoxy-16b-(2-fluoroethoxy)-fusidic acid (Compound 164),
24-bromo-16-deacetoxy-16b-(2-methoxyethoxy)-fusidic acid (Compound 165),
24-(trans-1-hexen-1-yl)-fusidic acid (Compound 166),
24-(trans-1-buten-3,3-dimethyl-1-yl)-fusidic acid (Compound 167),
24-(trans-1-nonen-1-yl)-fusidic acid (Compound 168),
24-(trans-5-chloro-1-penten-1-yl)-fusidic acid (Compound 169),
24-(trans-2-phenyl-1-vinyl)-fusidic acid (Compound 170),
24-(2-phenyl-1-ethyl)-fusidic acid (Compound 171),
24-(4-n-propylphenyl)-fusidic acid (Compound 172),
24-(4-vinylphenyl)-fusidic acid (Compound 173),
24-(4-tert-butylphenyl)-fusidic acid (Compound 174),
24-(4-cyanophenyl)-fusidic acid (Compound 175),
24-(3-biphenyl)-fusidic acid (Compound 176),
24-(4-(trifluoromethyl)phenyl)-fusidic acid (Compound 177),
24-(4-methoxyphenyl)-fusidic acid (Compound 178),
24-(3-cyanophenyl)-fusidic acid (Compound 179),
24-(2-methoxyphenyl)-fusidic acid (Compound 180),
24-(3-nitrophenyl)-fusidic acid (Compound 181),
24-(3-bromophenyl)-fusidic acid (Compound 182),
24-(4-(methylthio)phenyl)-fusidic acid (Compound 183),
24-(2-naphtyl)-fusidic acid (Compound 184),
24-(3,5-bis-(trifluoromethyl)phenyl)-fusidic acid (Compound 185),
24-(3,4-dimethoxyphenyl)-fusidic acid (Compound 186),
24-(3,5-dibromophenyl)-fusidic acid (Compound 187),
24-bromofusidic acid, cholin salt (Compound 188),
24-bromofusidic acid, L-arginine salt (Compound 189),
24-bromofusidic acid, 2-(dimethylamino)-ethanol salt (Compound 190),
24-bromofusidic acid, 4-(2-hydroxyethyl)-morpholin salt (Compound 191),
24-bromofusidic acid, L-lysine salt (Compound 192),
24-bromofusidic acid, N-(2-hydroxyethyl)-pyrrolidine salt (Compound 193),
24-bromofusidic acid, ethanolamine salt ( Compound 194),
24-bromofusidic acid, potassium salt (Compound 195),
24-bromofusidic acid, tetrabutylammonium salt (Compound 196),
24-bromofusidic acid, benzyltrimethylammonium salt (Compound 197),
24-bromofusidic acid, cetyltrimethylammonium salt (Compound 198),
24-bromofusidic acid, tetramethylammonium salt (Compound 199),
24-bromofusidic acid, tetrapropylammonium salt (Compound 300),
24-bromofusidic acid, tris(hydroxymethyl)aminomethane salt (Compound 301),
24-bromofusidic acid, N-methyl-D-glucamine salt (Compound 302),
24-bromofusidic acid, silver salt (Compound 303),
24-bromofusidic acid, benzethonium salt (Compound 304),
24-bromofusidic acid, triethanolamine salt (Compound 305),
24-(trans-1-hexen-1-yl)-fusidic acid pivaloyloxymethylester (Compound 306), with 24-(trans-1-buten-3,3-dimethyl-1-yl)-fusidic acid pivaloyloxymethyl ester (Compound 307),
24-(trans-1-nonen-1-yl)-fusidic acid pivaloyloxymethyl ester (Compound 308),
24-(trans-5-chloro-1-penten-1-yl)-fusidic acid pivaloyloxymethyl ester (Compound 309),
24-(trans-2-phenyl-1-vinyl)-fusidic acid pivaloyloxymethyl ester (Compound 310),
24-(2-phenyl-1-ethyl)-fusidic acid pivaloyloxymethyl ester (Compound 311),
24-(4-n-propylphenyl)-fusidic acid pivaloyloxymethyl ester (Compound 312),
24-(4-vinylphenyl)-fusidic acid pivaloyloxymethyl ester (Compound 313),
24-(4-tert-butylphenyl)-fusidic acid pivaloyloxymethyl ester (Compound 314),
24-(4-cyanophenyl)-fusidic acid pivaloyloxymethyl ester (Compound 315),
24-(3-biphenyl)-fusidic acid pivaloyloxymethyl ester (Compound 316),
24-(4-(trifluoromethyl)phenyl)-fusidic acid pivaloyloxymethyl ester (Compound 317),
24-(4-methoxyphenyl)-fusidic acid pivaloyloxymethyl ester (Compound 318),
24-(3-cyanophenyl)-fusidic acid pivaloyloxymethyl ester (Compound 319),
24-(2-methoxyphenyl)-fusidic acid pivaloyloxymethyl ester (Compound 320),
24-(3-nitrophenyl)-fusidic acid pivaloyloxymethyl ester (Compound 321),
24-(3-bromophenyl)-fusidic acid pivaloyloxymethyl ester (Compound 322),
24-(4-(methylthio)phenyl)-fusidic acid pivaloyloxymethyl ester (Compound 323),
24-(2-naphtyl)-fusidic acid pivaloyloxymethyl ester (Compound 324),
24-(3,5-bis-(trifluoromethyl)phenyl)-fusidic acid pivaloyloxymethyl ester (Compound 325),
24-(3,4-dimethoxyphenyl)-fusidic acid pivaloyloxymethyl ester (Compound 326), and
24-(3,5-dibromophenyl)-fusidic acid pivaloyloxymethyl ester (Compound 327).

21. A compound according to claim 1 for use in therapy.

22. A pharmaceutical composition comprising a compound according to claim 1 together with a pharmaceutically acceptable excipient or vehicle.

23. A pharmaceutical composition according to claim 22 further comprising another therapeutically active compound is selected from the group consisting of antibiotics and corticosteroids.

24. A pharmaceutical composition according to claim 23, wherein said other therapeutically active compound is selected from the group consisting of penicillins (phenoxymethyl penicillin, benzyl penicillin, dicloxacillin, ampicillin, amoxicillin, pivampicillin, flucloxacillin, piperacillin and mecellinam), cefalosporins (cefalexin, cefalotin, cefepim, cefotaxim, ceftazidim, ceftriazon and ceftiroxim), monobactams (aztreonam) and carbapenems (meropenem); macrolides (azithromycin, clarithromycin, erythromycin and roxithromycin); polymyxins (colistin); tetracyclins (tetracycline, doxycyclin, oxytetracyclin and lymecyclin); aminoglycosides (streptomycin, gentamicin, tobramycin and netilmicin); fluoroquinolones (norfloxacin, ofloxacin, ciprofloxacin and moxifloxacin); clindamycin, lincomycin, teicoplanin, vancomycin, oxazolidones (linezolid), rifamycin, metronidazol, fusidic acid, hydrocortisone, betamethason-17-valerate and triamcinolone acetonid.

25. A method of treating, preventing or ameliorating infections in a patient, the method comprising administering to said patient an effective amount of a compound according to claim 1, and optionally further comprising concomitant or sequential administration of one or more other therapeutically active compounds.

26. A method according to claim 25, wherein said other therapeutically active compound is selected from the group consisting of antibiotics and corticosteroids.

27. A method according to claim 25, wherein said other therapeutically active compound is selected from the group consisting of penicillins (phenoxymethyl penicillin, benzyl penicillin, dicloxacillin, ampicillin, amoxicillin, pivampicillin, flucloxacillin, piperacillin and mecellinam), cefalosporins (cefalexin, cefalotin, cefepim, cefotaxim, ceftazidim, ceftriazon and ceftiroxim), monobactams (aztreonam) and carbapenems (meropenem); macrolides (azithromycin, clarithromycin, erythromycin and roxithromycin); polymyxins (colistin); tetracyclins (tetracycline, doxycyclin, oxytetracyclin and lymecyclin); aminoglycosides (streptomycin, gentamicin, tobramycin and netilmicin); fluoroquinolones (norfloxacin, ofloxacin, ciprofloxacin and moxifloxacin); clindamycin, lincomycin, teicoplanin, vancomycin, oxazolidones (linezolid), rifamycin, metronidazol, fusidic acid, hydrocortisone, betamethason-17-valerate and triamcinolone acetonid.

28. A method according to claim 25, wherein said infection is a bacterial infection.

29. A method of preparing a compound of formula Ia, 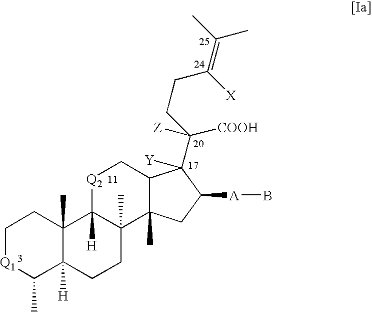
wherein X represents bromo, Y and Z both represent hydrogen, or together with the C-17/C-20 bond form a double bond between C-17 and C-20, or together are methylene and form a cyclopropane ring in combination with C-17 and C-20; A represents a bond, O, S or S(O); B represents C1-6 alkyl, C2-6 alkenyl, C1-6 acyl, C3-7 cycloalkylcarbonyl or benzoyl, all of which are optionally substituted with one or more substituents selected from the group consisting of halogen, hydroxy, C1-6 alkoxy, aryl, heterocyclyl and azido, or, if A represents a bond, B may also represent hydrogen; Q1 and Q2 independently represent —C(O)—, —(CHOH)—, —(CHSH)—, or —(CHW)—, wherein W represents halogen, cyano, azido or trifluoromethyl, the method comprising
(a) dissolving fusidic acid or a suitable fusidic acid analogue in a suitable organic solvent followed by treatment with bromine to give a 24,25-dibromo intermediate of general structure Ib, 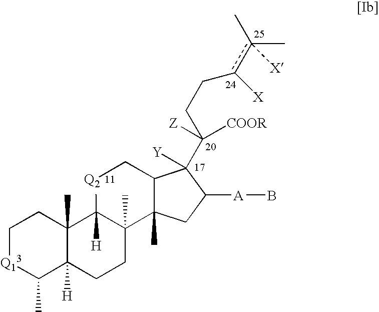
wherein X and X′ represent bromo, R is hydrogen, the bond between C-24 and C-25 is a single bond, and Y, Z, A, B, Q1, and Q2 are as defined above;
(b) treating a solution of the 24,25-dibromo intermediate in a suitable solvent in the presence of a suitable base to give the dehydrobrominated compound of formula Ia, in the form of a salt; and
(c) acidifying the salt generated in step (b) to obtain the compound of formula Ia in free acid form.

30. A compound of general structure Ib, 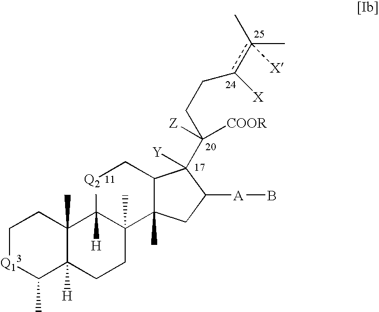
wherein X and X′ represent bromo, R is hydrogen, the bond between C-24 and C-25 is a single bond, Y and Z both represent hydrogen, or together with the C-17/C-20 bond form a double bond between C-17 and C-20, or together are methylene and form a cyclopropane ring in combination with C-17 and C-20, A represents a bond, O, S or S(O); B represents C1-6 alkyl, C2-6 alkenyl, C1-6 acyl, C3-7 cycloalkylcarbonyl or benzoyl, all of which are optionally substituted with one or more substituents selected from the group consisting of halogen, hydroxy, C1-6 alkoxy, aryl, heterocyclyl and azido, or, if A represents a bond, B may also represent hydrogen; Q1 and Q2 independently represent —C(O)—, —(CHOH)—, —(CHSH)—, or —(CHW)—, wherein W represents halogen, cyano, azido or trifluoromethyl.

31-35. (canceled)
